(19) United States
(12) Patent Application Publication
(10) Pub. No.: US 2024/0252654 A1
(43) Pub. Date: Aug. 1, 2024

(54) IMPROVED SMALL MOLECULES

(71) Applicants: University of Dundee, Dundee, Tayside (GB); Promega Corporation, Madison, WI (US)

(72) Inventors: Alessio Ciulli, Dundee, Tayside (GB); Andrea Testa, Dundee, Tayside (GB); Chiara Maniaci, Dundee, Tayside (GB); Nikolai Makukhin, Dundee, Tayside (GB); Satomi Imaide, Dundee, Tayside (GB); Danette Daniels, Madison, WI (US); Kristin Riching, Madison, WI (US)

(21) Appl. No.: 18/252,083

(22) PCT Filed: Oct. 19, 2021
(86) PCT No.: PCT/GB2021/052698

§ 371 Date: May 8, 2023

(30) Foreign Application Priority Data
Nov. 10, 2020 (GB) 2017717.6

(51) Int. Cl. A61K 47/55; A61K 47/60

(52) U.S. Cl. CPC A61K 47/55; A61K 47/60

(57) ABSTRACT

This invention particularly relates to trifunctional PROTACs of formula I as described herein which bind to a protein within the Bromo- and Extra-Terminal (BET) family of proteins, and especially to PROTACs including small molecule E3 ubiquitin ligase protein binding ligand compounds which induce preferential degradation of the BRD2 protein within the bromodomain of the BET family of proteins.

### FIELD OF THE INVENTION

This invention relates to novel small molecule E3 ubiquitin ligase protein binding ligand compounds, and to their utility in Proteolysis Targeted Chimeras (PROTACs), as well as processes for the preparation thereof, and use in medicine. This invention particularly relates to PROTACs which bind to a protein within the Bromo- and Extra-Terminal (BET) family of proteins, and especially to PROTACs including small molecule E3 ubiquitin ligase protein binding ligand compounds which induce preferential degradation of the BRD2 protein within the bromodomain of the BET family of proteins.

### BACKGROUND OF THE INVENTION

Targeted degradation compounds, conventionally classified as either PROTACs or molecular glues depending on whether bivalent or monovalent for binding, have shown great promise as a new class of chemical probes to study biology and therapeutics for treatment of disease¹⁻³.

Degraders form a ternary complex, bringing together the target protein and an E3 ubiquitin ligase component, resulting in ubiquitination and subsequent degradation of the target protein via the proteasome.

Molecular glues typically engage monovalently eitherthe E3 ligase orthe target, and then adventitiously induce highly cooperative recruitment of the non-cognate partner in a ternary complex^{4-8.}

In contrast, PROTACs are traditionally conceived as bifunctional, i.e. composed of two ligands joined by a linker, and as a result can engage the target or E3 component separately in binary complexes, or both target and E3 component simultaneously to form a ternary complex⁹⁻¹¹. This design feature allows PROTACs not only a wider target scope than molecular glues but also the ability to standardize design with the use of established E3 ligase binders. Indeed, potent PROTAC degraders have been developed to most commonly recruit either the von Hippel-Lindau (VHL) or cereblon (CRBN) E3 ligase against a wide spectrum of protein targets including nuclear^{12 13 14 15, 16}, cytoplasmic^{11, 17, 18}, membrane-bound¹⁹, and multi-pass transmembrane proteins²⁰.

Due to their complex mode of action via the ternary complex, PROTACs have shown unexpected advantages compared to the inhibitors they are composed of. For example, PROTACs can discriminate amongst highly homologue targets²¹⁻²⁴, and can exhibit much greater potencies than expected, due to a catalytic mechanism of action, which can compensate for low binary binding affinities or poor cellular permeability, and allow for use of weak, non-functional inhibitors to serve as warhead ligands^{15, 22, 25, 26} Furthermore, PROTACs can impact proteins in complexes even though not directly engaged by the degrader via either bystander ubiquitination or destabilization^{15, 27, 28}. Despite the aforementioned advantages and remarkable successes demonstrated, it can be challenging to design PROTACs efficiently, often requiring extensive chemical optimization^{29, 30}.

Unlike inhibitors, degraders must function beyond simple binary engagement. Instead they must work throughout a cascade of events, not only inducing proximity between two proteins which do not natively interact, but also yielding a productive ternary complexwhich structurally positions the target protein for efficient ubiquitination by the E3 ligase^{31 32, 33}.

Recent X-ray crystallographic structures and allied biophysical studies of PROTAC ternary complexes have elegantly demonstrated that some PROTAC-mediated ternary complexes are, like molecular glues, capable of cooperative binding, most notably shown for VHL-MZ1-BRD4-BD2 ternary complex²¹. This and subsequent studies have shown how in order to drive productive target ubiquitination and profound target degradation at catalytic low concentrations, degraders need to form complexes of sufficient stability, cooperativity and residence time, enhanced by favourable intra-complex interactions^{15, 21, 22, 31, 32}.

Such optimal “gluing” of molecular recognition features can be challenging to realise with conventional PROTAC degraders that are by definition monovalent at the target of interest. This limits their ability to leverage favourable protein-protein and other stabilizing interactions within the ternary complex.

Indeed, non-cooperative or even negatively cooperative PROTAC ternary complexes are often observed, which albeit permissive to downstream protein ubiquitination and degradation, can lead to unfavourable pharmacological properties of degraders such as pronounced hook effect at higher concentration, often resulting in slow and incomplete target degradation^{17, 34}.

It is an object of the present invention to mitigate or address at least some of these problems.

### SUMMARY OF THE INVENTION

The inventors have developed a new strategy exploiting trivalent PROTACs to boost targeted protein degradation of the Bromo and Extra Terminal (BET) domain family member proteins BRD2, BRD3 and BRD4. The inventors have developed a strategy to synergize the effects of a bivalent target ligand with E3 ligase recruitment to produce trivalent PROTACs with enhanced avidity, cooperativity and target degradation.

In a first aspect there is provided a compound of Formula I:

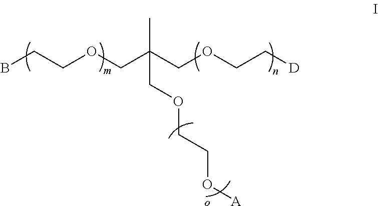

wherein B and D are each a ligand which binds to a target protein or polypeptide which is to be degraded by ubiquitin ligase;
wherein A is an E3 ubiquitin ligase protein binding ligand;
wherein m, n and o are each independently selected from 0, 1, 2, 3, 4, 5 and 6;
or a pharmaceutically acceptable, salt, enantiomer, stereoisomer, hydrate, solvate, or polymorph thereof.

In the compounds of formula I, m and n may each be independently selected from 2, 3, 4, 5 and 6. In preferred compounds of formula I, m and n may each be independently selected from 2, 3 and 4.

In the compounds of formula I, m and n may both be the same and selected from 2, 3, 4, 5 and 6. In preferred compounds of formula I, m and n may both be the same and selected from 2, 3 and 4. In further preferred compounds of formula I, m and n may both be 3.

In the compounds of formula I, o may be selected from 0 and 1. In preferred compounds of formula I, o may be 0.

In the compounds of formula I, B and D may each be a chemical moiety which binds to a protein within the bromo- and Extra-terminal (BET) family of proteins. In the compounds of formula I, B and D may each be a chemical moiety which induces degradation of the BRD2, BRD3, and/or BRD4 proteins within the bromo- and Extra-terminal (BET) family of proteins.

In the compounds of formula I, B and D may each be independently selected from:

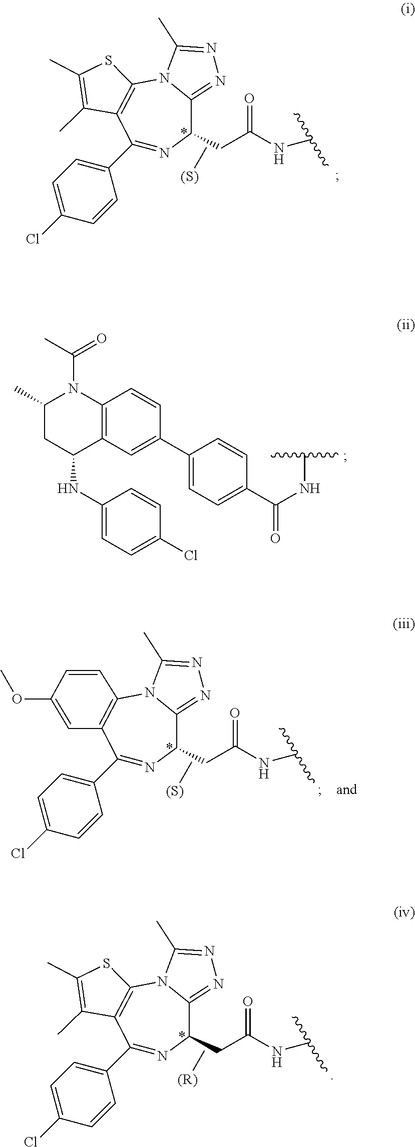

In preferred embodiments, at least one of B and D is

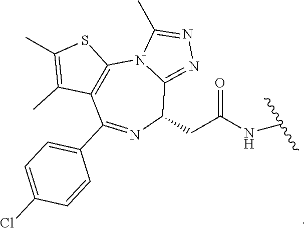 [SMILES: *NC(=O)C[C@@H]1N=C(c2ccc(Cl)cc2)c2c(sc(C)c2C)-n2c(C)nnc21]

In preferred embodiments, the chiral centre of

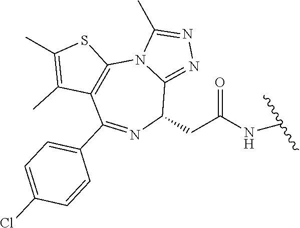 [SMILES: *NC(=O)C[C@@H]1N=C(c2ccc(Cl)cc2)c2c(sc(C)c2C)-n2c(C)nnc21]

has S configuration. In some embodiments, one of B or D is

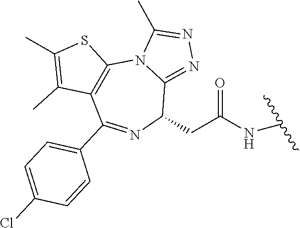 [SMILES: *NC(=O)C[C@@H]1N=C(c2ccc(Cl)cc2)c2c(sc(C)c2C)-n2c(C)nnc21]

and the other of B or D is

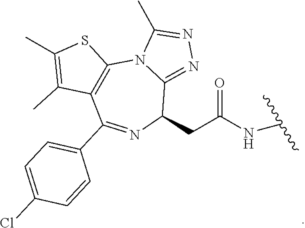 [SMILES: *NC(=O)C[C@H]1N=C(c2ccc(Cl)cc2)c2c(sc(C)c2C)-n2c(C)nnc21]

That is, in some embodiments one of B or D is

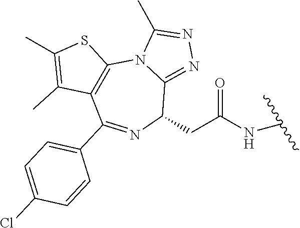 [SMILES: *NC(=O)C[C@@H]1N=C(c2ccc(Cl)cc2)c2c(sc(C)c2C)-n2c(C)nnc21]

having S configuration at the chiral centre and the other of B or D is

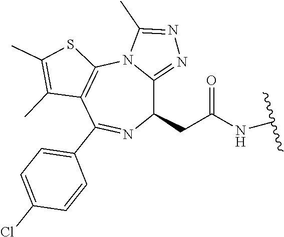 [SMILES: *NC(=O)C[C@H]1N=C(c2ccc(Cl)cc2)c2c(sc(C)c2C)-n2c(C)nnc21]

with having R configuration at the chiral centre. In most preferred embodiments, both B and D are

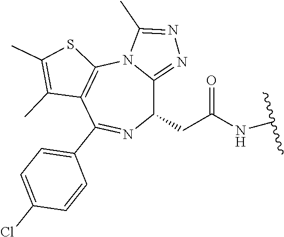 [SMILES: *NC(=O)C[C@@H]1N=C(c2ccc(Cl)cc2)c2c(sc(C)c2C)-n2c(C)nnc21]

(i.e. both B and D are

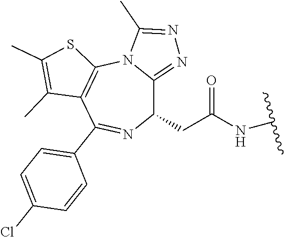 [SMILES: *NC(=O)C[C@@H]1N=C(c2ccc(Cl)cc2)c2c(sc(C)c2C)-n2c(C)nnc21]

having S configuration at the chiral centre.

In the compound of formula I, A may be selected from a von Hippel-Lindau (VHL)-E3 ubiquitin ligase binding ligand or a cereblon (CRBN)-E3 ubiquitin ligase ligand.

In preferred embodiments of the compound of formula I, A may be selected from:

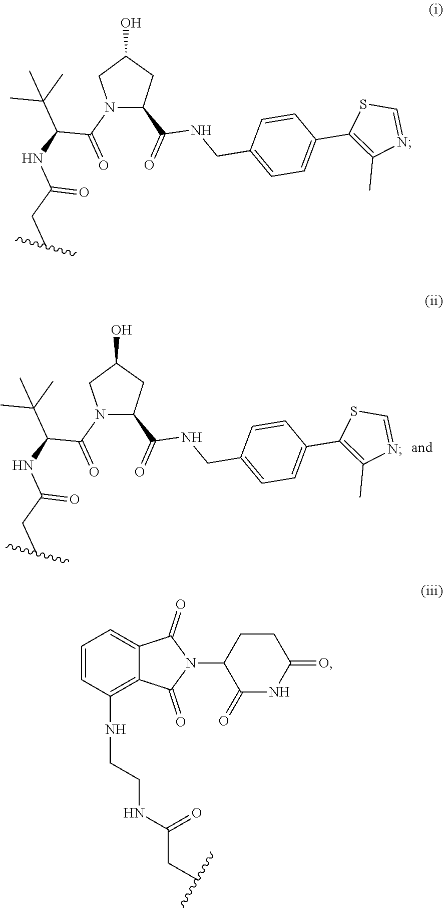 [SMILES: *CC(=O)N[C@H](C(=O)N1C[C@H](O)C[C@H]1C(=O)NCc1ccc(-c2scnc2C)cc1)C(C)(C)C ; *CC(=O)N[C@H](C(=O)N1C[C@@H](O)C[C@H]1C(=O)NCc1ccc(-c2scnc2C)cc1)C(C)(C)C ; *CC(=O)NCCNc1cccc2c1C(=O)N(C1CCC(=O)NC1=O)C2=O]

In further preferred embodiments of the compound of formula I, A may be selected from:

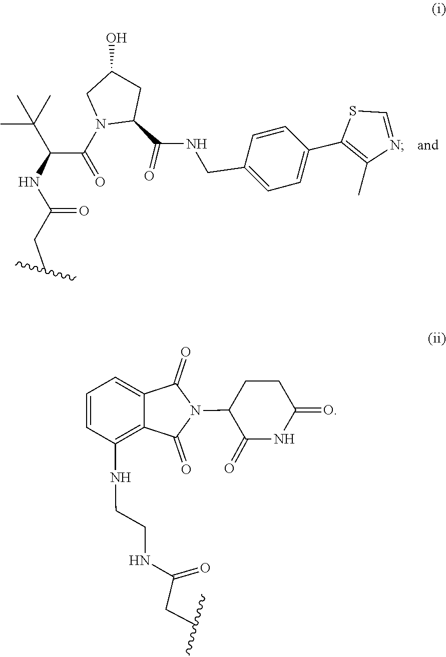 [SMILES: *CC(=O)N[C@H](C(=O)N1C[C@H](O)C[C@H]1C(=O)NCc1ccc(-c2scnc2C)cc1)C(C)(C)C ; "and" ; *CC(=O)NCCNc1cccc2c1C(=O)N(C1CCC(=O)NC1=O)C2=O]

In even further preferred embodiments, A may be

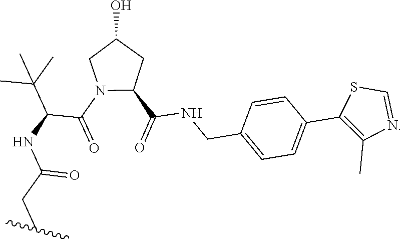 [SMILES: *CC(=O)N[C@H](C(=O)N1C[C@H](O)C[C@H]1C(=O)NCc1ccc(-c2scnc2C)cc1)C(C)(C)C]

The compounds of formula I may have formula IA:

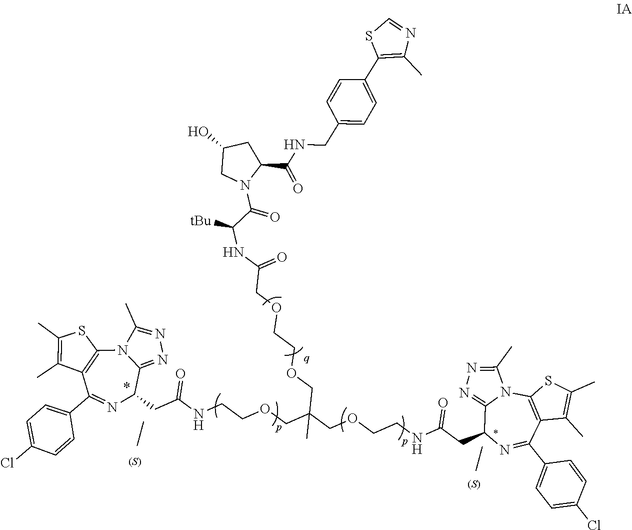

wherein p is selected from 2, 3, 4, 5 and 6;

wherein q is selected from 0, 1, and 2;

or a pharmaceutically acceptable, salt, enantiomer, stereoisomer, hydrate, solvate, or polymorph thereof.

In preferred compounds of formula IA, p may be selected from 2, 3 and 4. In further preferred compounds of formula IA, p may be 3.

In preferred compounds of formula IA, q may be selected from 0 and 1. In further preferred compounds of formula IA, q may be 0.

The compounds of formula I may have formula IB:

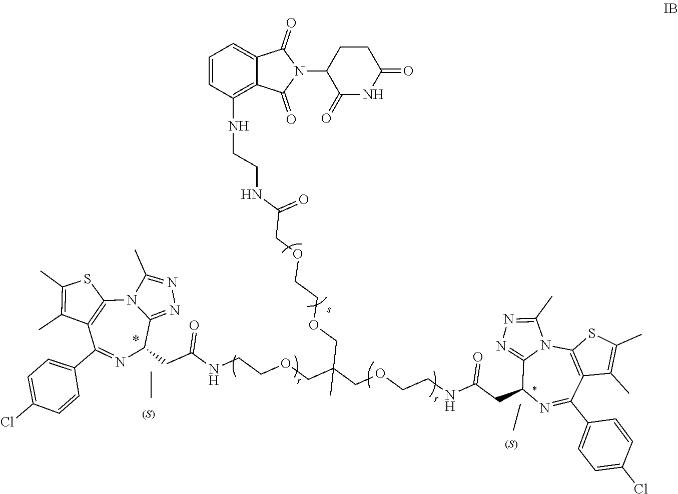

wherein r is selected from 2, 3, 4, 5 and 6;

wherein s is selected from 0, 1, and 2;

or a pharmaceutically acceptable, salt, enantiomer, stereoisomer, hydrate, solvate, or polymorph thereof.

In preferred compounds of formula IB, r may be selected from 2, 3 and 4. In further preferred compounds of formula IB, r may be 3.

In preferred compounds of formula IB, s may be selected from 0 and 1. In further preferred compounds of formula IB, s may be 0.

The compounds of Formula I may have formula IC:

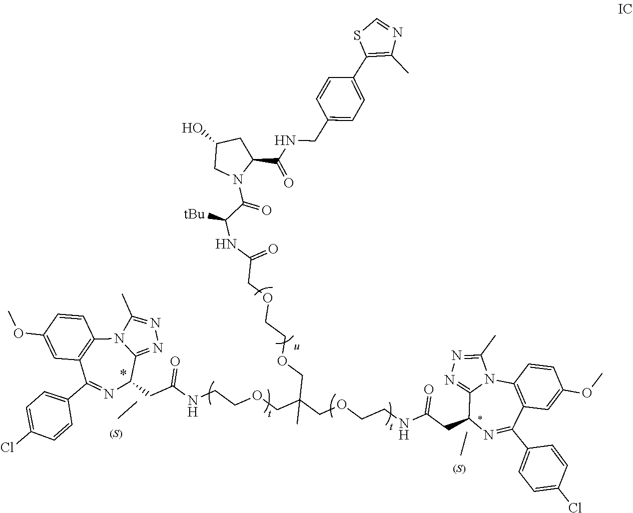

wherein t is selected from 2, 3, 4, 5 and 6;

wherein u is selected from 0, 1, and 2;

or a pharmaceutically acceptable, salt, enantiomer, stereoisomer, hydrate, solvate, or polymorph thereof.

In preferred compounds of formula IC, t may be selected from 2, 3 and 4. In further preferred compounds of formula IC, t may be 3.

In preferred compounds of formula IC, u may be selected from 0 and 1. In further preferred compounds of formula IC, u may be 0.

The compounds of formula I may have formula ID:

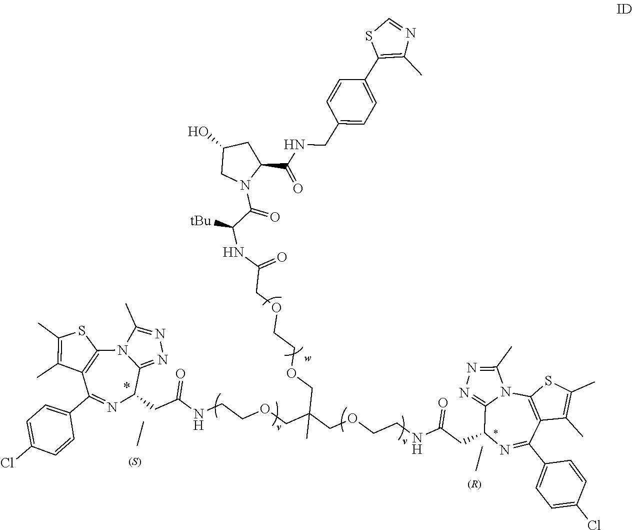

wherein v is selected from 2, 3, 4, 5, and 6;

wherein w is selected from 0, 1, and 2;

or a pharmaceutically acceptable, salt, enantiomer, stereoisomer, hydrate, solvate, or polymorph thereof.

In preferred compounds of formula ID, v may be selected from 2, 3 and 4. In further preferred compounds of formula ID, v may be 3.

In preferred compounds of formula ID, w may be selected from 0 and 1. In further preferred compounds of formula ID, w may be 0.

The compounds of formula I, IA, IB, IC, and ID may be selected from:
(i) N,N′-(11-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(ii) N,N′-(11-((2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(iii) N,N′-(14-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-14-methyl-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(iv) N,N′-(11-((2-((2-((2-(2,6-dioxopiperidin-3-yl)-1,3-dioxoisoindolin-4-yl)amino)ethyl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(v) N,N′-(11-((2-(2-((2-((2-(2,6-dioxopiperidin-3-yl)-1,3-dioxoisoindolin-4-yl)amino)ethyl)amino)-2-oxoethoxy)ethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(vi) N,N′-(14-((2-((2-((2-(2,6-dioxopiperidin-3-yl)-1,3-dioxoisoindolin-4-yl)amino)ethyl)amino)-2-oxoethoxy)methyl)-14-methyl-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(vii) N,N′-(11-((2-(((S)-1-((2S,4S)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(viii) (2S,4R)-1-((20S)-20-(tert-butyl)-1-((R)-4-(4-chlorophenyl)-2,3,9-trimethyl-6H-thieno[3,2-f][1,2,4]triazolo[4,3-a][1,4]diazepin-6-yl)-14-(13-((S)-4-(4-chlorophenyl)-2,3,9-trimethyl-6H-thieno[3,2-f][1,2,4]triazolo[4,3-a][1,4]diazepin-6-yl)-12-oxo-2,5,8-trioxa-11-azatridecyl)-14-methyl-2,18-dioxo-6,9,12,16-tetraoxa-3,19-diazahenicosan-21-oyl)-4-hydroxy-N-(4-(4-methylthiazol-5-yl)benzyl)pyrrolidine-2-carboxamide;
(ix) N,N′-(8-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-8-methyl-3,6,10,13-tetraoxapentadecane-1,15-diyl)bis(2-((S)-4-(4-chlorophenyl)-2,3,9-trimethyl-6H-thieno[3,2-f][1,2,4]triazolo[4,3-a][1,4]diazepin-6-yl)acetamide);

or a pharmaceutically acceptable, salt, enantiomer, stereoisomer, hydrate, solvate, or polymorph thereof.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts suitable for use herein include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange.

Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulphonate.

The PROTAC compounds of the invention can be administered as pharmaceutically acceptable prodrugs which release the compounds of the invention in vivo. “Prodrug”, as used herein means a compound which is convertible in vivo by metabolic means (e.g. by hydrolysis) to afford any compound delineated by the formulae of the instant invention. Various forms of prodrugs are known in the art, for example, as discussed in “Design and Application of Prodrugs, Textbook of Drug Design and Development, Chapter 5, 113-191 (1991); Bundgaard, et al., Journal of Drug Deliver Reviews, 8:1-38(1992); and Bernard Testa and Joachim Mayer, “Hydrolysis In Drug and Prodrug Metabolism—Chemistry, Biochemistry and Enzymology,” John Wiley and Sons, Ltd. (2003).

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Related terms are to be interpreted accordingly in line with the definitions provided above and the common usage in the technical field.

The compounds of formula I for use in the PROTAC compounds of Formula I as defined herein may be represented as a defined stereoisomer. The absolute configuration of such compounds can be determined using art-known methods such as, for example, X-ray diffraction or NMR and/or implication from starting materials of known stereochemistry.

Pharmaceutical compositions in accordance with the invention will preferably comprise substantially stereoisomerically pure preparations of the indicated stereoisomer.

Pure stereoisomeric forms of the compounds and intermediates as mentioned herein are defined as isomers which are substantially free of other enantiomeric or diastereomeric forms of the same basic molecular structure of said compounds or intermediates. In particular, the term “stereoisomerically pure” concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates as detailed herein may be obtained by the application of art-known procedures. For instance, enantiomers may be separated from each other by the selective crystallization of their diastereomeric salts with optically active acids or bases. Examples thereof are tartaric acid, dibenzoyl tartaric acid, ditoluoyltartaric acid and camphorsulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereo-specifically. Preferably, if a specific stereoisomer is desired, said compound is synthesized by stereospecific methods of preparation. These methods will advantageously employ enantiomerically pure starting materials.

The diastereomeric racemates of the compounds of formula I for use in the PROTAC compounds of Formula I as defined herein can be obtained separately by conventional methods. Appropriate physical separation methods that may advantageously be employed are, for example, selective crystallization and chromatography, e.g. column chromatography.

The present invention provides PROTAC compounds Formula I wherein B is a chemical moiety which binds to a protein within the bromo- and Extra-terminal (BET) family of proteins, preferably PROTAC compounds of Formula I wherein B is a chemical moiety which binds to a protein within the bromo- and Extra-terminal (BET) family of proteins independently selected from: BRD2, BRD3 and BRD4, and particularly PROTAC compounds of Formula I wherein B is a chemical moiety which selectively induces degradation of the BRD2 protein within the bromo- and Extra-terminal (BET) family of proteins.

In a further aspect, the invention provides a PROTAC compound of formula I as defined herein for use as a medicament.

The term “subject” as used herein refers to a mammal. A subject therefore refers to, for example, dogs, cats, horses, cows, pigs, guinea pigs, and the like. Preferably the subject is a human. When the subject is a human, the subject may also be referred to herein as a patient.

“Treat”, “treating” and “treatment” refer to a method of alleviating or abating a disease and/or its attendant symptoms.

The term “therapeutically effective amount” means an amount effective to treat, cure or ameliorate a disease, condition illness or sickness.

A further aspect of the invention provides a method for the prophylaxis or treatment of a disease or condition associated with deregulation of protein activity of one or more proteins within the Bromo- and Extra-terminal (BET) family of proteins BRD2, BRD3 and BRD4 comprising the administration of a PROTAC compound of Formula I to a subject suffering from or likely to be exposed to said disease or condition. A related aspect of the invention provides the use of a PROTAC compound of Formula I in the treatment or prophylaxis of a disease or condition associated with deregulation of BET protein activity. A further related aspect provides the use of a PROTAC compound of Formula I as defined herein for the treatment or prophylaxis of a disease or condition associated with deregulation of BET protein activity.

A further aspect of the invention provides a method for the prophylaxis or treatment of a disease or condition associated with deregulation of protein activity of one or more proteins within the Bromo- and Extra-terminal (BET) family of proteins BRD2, BRD3 and BRD4 comprising the administration of a therapeutically effective amount of a PROTAC compound of Formula I to a subject suffering from or likely to be exposed to said disease or condition. A related aspect of the invention provides the use of a therapeutically effective amount of a PROTAC compound of Formula I in the treatment or prophylaxis of a disease or condition associated with deregulation of BET protein activity. A further related aspect provides the use of a therapeutically effective amount of a PROTAC compound of Formula I as defined herein for the treatment or prophylaxis of a disease or condition associated with deregulation of BET protein activity.

A further aspect of the invention provides a method for the prophylaxis or treatment of a disease or condition associated with selective degradation of the BRD2 protein within the bromodomain of the BET family of proteins comprising the administration of a PROTAC compound of Formula I as defined herein to a subject suffering from or likely to be exposed to said disease or condition. A related aspect of the invention provides the use of a PROTAC compound of Formula I in the treatment or prophylaxis of a disease or condition associated with selective degradation of the BRD2 protein within the bromodomain of the BET family of proteins. A further related aspect provides the use of a PROTAC compound of Formula I for the treatment or prophylaxis of a disease or condition associated with selective degradation of the BRD2 protein within the bromodomain of the BET family of proteins.

Diseases or conditions associated with deregulation of protein activity of one or more proteins within the Bromo- and Extra-terminal (BET) family of proteins BRD2, BRD3 and BRD4 which may be treated via the administration of a PROTAC compound of Formula I as defined herein include: cancer; benign proliferative disorders; infectious or non-infectious inflammatory events; autoimmune diseases; inflammatory diseases; systemic inflammatory response syndromes; viral infections and diseases; and ophthalmological conditions.

The present invention also provides PROTAC compounds of Formula I in accordance with any aspect, or preferred aspect detailed herein for use in medicine, particularly for use in conditions or diseases where binding to a protein within the bromo- and Extra-terminal (BET) family of proteins independently selected from: BRD2, BRD3 and BRD4 is implicated, and especially for use in the treatment of one or more conditions or diseases independently selected from: cancer; benign proliferative disorders; infectious or non-infectious inflammatory events; autoimmune diseases; inflammatory diseases; systemic inflammatory response syndromes; viral infections and diseases; and ophthalmological conditions.

There is also provided herein PROTAC compounds of Formula I in accordance with any aspect herein, for use in the treatment of cancer and a method of treatment of cancer by administration of an effective amount of a PROTAC compound of Formula I in accordance with any aspect herein, to a mammal, in particular a human in need of such treatment.

Cancer-types which may be treated via the administration of a PROTAC compound of Formula I as defined herein include: carcinoma-type cancers associated with epithelial cells disorders such as for example breast cancer, prostate cancer, lung cancer pancreatic cancer and cancer of the colon; sarcoma-type cancers associated with mesenchymal cell disorders; lymphoma; leukaemia, such as for example acute myeloid leukaemia; cancers and/or cancerous tumours associated with pluripotent cells such as testicular cancer and ovarian carcinoma.

Examples of cancers that the compounds of the present invention may be used in the treatment of include: adrenal cancer, acinic cell carcinoma, acoustic neuroma, acral lentigious melanoma, acrospiroma, acute eosinophilic leukaemia, acute erythroid leukaemia, acute lymphoblastic leukaemia, acute megakaryoblastic leukaemia, acute monocytic leukaemia, acute promyelocytic leukaemia, adenocarcinoma, adenoid cystic carcinoma, adenoma, adenomatoid odontogenic tumor, adenosquamous carcinoma, adipose tissue neoplasm, adrenocortical carcinoma, adult T-cell leukaemia/lymphoma, aggressive NK-cell leukaemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukaemia, B-cell prolymphocytic leukaemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, oesophageal cancer, foetus in feta, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukaemia, hemangioblastoma, head and neck cancer, hemangiopericytoma, haematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukaemia, leydig cell tumor, liposarcoma, lung cancer, lymphangioma, lymphangiosarcoma, lymphoepithelioma, lymphoma, acute lymphocytic leukaemia, acute myelogeous leukaemia, chronic lymphocytic leukaemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukaemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumours, small cell carcinoma, softtissue sarcoma, somatostatinoma, sootwart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, Wilms' tumor, haematological cancers (such as leukaemia), epithelial cancers including lung, breast and colon carcinomas, midline carcinomas, mesenchymal, hepatic, renal and neurological tumours. Thus

Examples of benign proliferative disorders that the compounds of the present invention may be used in the treatment of include, but are not limited to, benign soft tissue tumours, bone tumours, brain and spinal tumours, eyelid and orbital tumours, granuloma, lipoma, meningioma, multiple endocrine neoplasia, nasal polyps, pituitary tumours, prolactinoma, pseudotumor cerebri, seborrheic keratoses, stomach polyps, thyroid nodules, cystic neoplasms of the pancreas, haemangiomas, vocal cord nodules, polyps, and cysts, Castleman disease, chronic pilonidal disease, dermatofibroma, pillar cyst, pyogenic granuloma, and juvenile polyposis syndrome.

There is also provided herein PROTAC compounds of Formula I in accordance with any aspect herein, for use in the treatment of infectious and non-infectious inflammatory events and autoimmune and other inflammatory diseases, disorders and syndromes and a method of treatment of infectious and non-infectious inflammatory events and autoimmune and other inflammatory diseases disorders and syndromes by administration of an effective amount of a PROTAC compound of Formula I in accordance with any aspect herein, to a mammal, in particular a human in need of such treatment. Examples of infectious and non-infectious inflammatory events and autoimmune and other inflammatory diseases, disorders and syndromes that the compounds of the present invention may be used in the treatment of include but are not limited to: inflammatory pelvic disease (PID), gout, pleurisy, eczema, splenitis, laryngitis, thyroiditis, prostatitis, pharyngitis, sarcoidosis, seborrheic dermatitis, irritable bowel syndrome (IBS), diverticulitis, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergic reactions, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anaemia, glomerulonephritis, dermatomyositis, multiple sclerosis, some myopathies, scleroderma, vasculitis, autoimmune haemolytic and thrombocytopenic states, Goodpasture's syndrome, atherosclerosis, Addison's disease, Parkinson's disease, Alzheimer's disease, Type I diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituatarism, Guillain-Barre syndrome, Behcet's disease, scleracierma, mycosis fungoides, acute inflammatory responses (such as acute respiratory distress syndrome and ischemia/reperfusion injury), and Graves' disease.

In other embodiments, the present invention provides PROTAC compounds of formula I in accordance with any aspect herein, for use in the treatment of systemic inflammatory response syndromes, and a method of treatment of systemic inflammatory response syndromes by administration of an effective amount of a PROTAC compound of formula I in accordance with any aspect herein, to a mammal, in particular a human in need of such treatment. Examples of systemic inflammatory response syndromes that the compounds of the present invention may be used in the treatment of include: LPS-induced endotoxic shock and/or bacteria-induced sepsis.

Autoimmune diseases and Autoimmune-related diseases which may be treated via the administration of a PROTAC compound of structure I as defined herein include: acute Disseminated Encephalomyelitis (ADEM); acute necrotizing hemorrhagic leukoencephalitis; Addison's disease; agammaglobulinemia; alopecia areata; amyloidosis; ankylosing spondylitis; anti-GBM/anti-TBM nephritis; antiphospholipid syndrome (APS); autoimmune angioedema; autoimmune aplastic anemia; autoimmune dysautonomia; autoimmune hepatitis; autoimmune hyperlipidemia; autoimmune immunodeficiency; autoimmune inner ear disease (AIED); autoimmune myocarditis; autoimmune oophoritis; autoimmune pancreatitis; autoimmune retinopathy; autoimmune thrombocytopenic purpura (ATP); autoimmune thyroid disease; autoimmune urticaria; axonal & neuronal neuropathies; Balo disease; Behcet's disease; bullous pemphigoid; cardiomyopathy; Castleman disease; celiac disease; Chagas disease; chronic fatigue syndrome; chronic inflammatory demyelinating polyneuropathy (CIDP); chronic recurrent multifocal ostomyelitis (CRMO); Churg-Strauss syndrome; cicatricial pemphigoid/benign mucosal pemphigoid; Crohn's disease; Cogans syndrome; cold agglutinin disease; congenital heart block; Coxsackie myocarditis; CREST disease; essential mixed cryoglobulinemia; demyelinating neuropathies; dermatitis herpetiformis; dermatomyositis; Devic's disease (neuromyelitis optica); discoid lupus; Dressier's syndrome; endometriosis; eosinophilic esophagitis; eosinophilic fasciitis; erythema nodosum; experimental allergic encephalomyelitis; Evans syndrome; fibromyalgia; fibrosing alveolitis; giant cell arteritis (temporal arteritis); giant cell myocarditis; glomerulonephritis; Goodpasture's syndrome; granulomatosis with polyangiitis (GPA) (formerly called Wegener's Granulomatosis); Graves' disease; Guillain-Barre syndrome; Hashimoto's encephalitis; Hashimoto's thyroiditis; hemolytic anemia; Henoch-Schonlein purpura; herpes gestationis; hypogammaglobulinemia; idiopathic thrombocytopenic purpura (ITP); IgA nephropathy; IgG4-related sclerosing disease; immunoregulatory lipoproteins; inclusion body myositis; interstitial cystitis; juvenile arthritis; juvenile diabetes (Type 1 diabetes); juvenile myositis; Kawasaki syndrome; Lambert-Eaton syndrome; leukocytoclastic vasculitis; lichen planus; lichen sclerosus; ligneous conjunctivitis; linear IgA disease (LAD); lupus (SLE); Lyme disease; Meniere's disease; microscopic polyangitis; mixed connective tissue disease (MCTD); Mooren's ulcer; Mucha-Habermann disease; multiple sclerosis; myasthenia gravis; myositis; narcolepsy; neuromyelitis optica (Devic's disease); neutropenia; ocular cicatricial pemphigoid; optic neuritis; palindromic rheumatism; PANDAS (Pediatric Autoimmune Neuropsychiatric Disorders Associated with Streptococcus); paraneoplastic cerebellar degeneration; paroxysmal nocturnal hemoglobinuria (PNH); Parry Romberg syndrome; Parsonnage-Turner syndrome; pars planitis (peripheral uveitis); pemphigus; peripheral neuropathy; perivenous encephalomyelitis; pernicious anemia; POEMS syndrome; polyarteritis nodosa; type I, II, & III autoimmune polyglandular syndromes; polymyalgia rheumatica; polymyositis; postmyocardial infarction syndrome; postpericardiotomy syndrome; progesterone dermatitis; primary biliary cirrhosis; primary sclerosing cholangitis; psoriasis; psoriatic arthritis; idiopathic pulmonary fibrosis; pyoderma gangrenosum; pure red cell aplasia; Raynauds phenomenon; reactive arthritis; reflex sympathetic dystrophy; Reiter's syndrome; relapsing polychondritis; restless legs syndrome; retroperitoneal fibrosis; rheumatic fever; rheumatoid arthritis; sarcoidosis; Schmidt syndrome; scleritis; scleroderma; Sjogren's syndrome; sperm & testicular autoimmunity; stiff person syndrome; subacute bacterial endocarditis (SBE); Susac's syndrome; sympathetic ophthalmia; Takayasu's arteritis; temporal arteritis/giant cell arteritis; thrombocytopenic purpura (TTP); Tolosa-Hunt syndrome; transverse myelitis; type 1 diabetes; ulcerative colitis; undifferentiated connective tissue disease (UCTD); uveitis; vasculitis; vesiculobullous dermatosis; vitiligo; Wegener's granulomatosis (now termed Granulomatosis with Polyangiitis (GPA).

As will be readily appreciated by the skilled person there is a certain degree of overlap between conditions and diseases within those defined herein as inflammatory and autoimmune disorders or conditions, which is to be expected in view of the complex nature of such conditions and the presentations of each individual subject.

There is additionally provided herein PROTAC compounds of Formula I in accordance with any aspect herein, for use in the treatment of viral infections and diseases, and a method of treatment of viral infections and diseases by administration of an effective amount of a PROTAC compound of formula I in accordance with any aspect herein, to a mammal, in particular a human in need of such treatment. Examples of viral infections and diseases that the compounds of the present invention may be used in the treatment of include: episome-based DNA viruses including, but not limited to, human papillomavirus, Herpesvirus, Epstein-Barr virus, human immunodeficiency virus, hepatis B virus, and hepatitis C virus.

There is also provided herein PROTAC compounds of formula I in accordance with any aspect herein, for use in the treatment of viral infections and a method of treatment of viral infections by administration of an effective amount of a PROTAC compound of formula I in accordance with any aspect herein, to a mammal, in particular a human in need of such treatment. Examples of viral infections that the compounds of the present invention may be used in the treatment of include herpes virus, human papilloma virus, adenovirus, poxvirus and other DNA viruses.

There is also provided herein PROTAC compounds of formula I in accordance with any aspect herein, for use in the treatment of ophthalmological indications and a method of treatment of ophthalmological indications by administration of an effective amount of a PROTAC compound of formula I in accordance with any aspect herein, to a mammal, in particular a human in need of such treatment. Examples of ophthalmological indications that the compounds of the present invention may be used in the treatment of include dry eye.

A further aspect of the invention provides a method for the prophylaxis or treatment of a disease or condition associated with deregulation of BET protein activity comprising the administration of a PROTAC compound of structure I as defined herein to a subject suffering from or likely to be exposed to said disease or condition wherein said disease or condition is independently selected from: cancer; benign proliferative disorders; infectious or non-infectious inflammatory events; autoimmune diseases; inflammatory diseases; systemic inflammatory response syndromes; viral infections and diseases; and ophthalmological conditions. A related aspect of the invention provides the use of a PROTAC compound of Formula I as defined herein in the treatment or prophylaxis of a disease or condition associated with deregulation of BET protein activity wherein said disease or condition is independently selected from: cancer; benign proliferative disorders; infectious or non-infectious inflammatory events; autoimmune diseases; inflammatory diseases; systemic inflammatory response syndromes; viral infections and diseases; and ophthalmological conditions. A further related aspect provides the use of a PROTAC compound of Formula I as defined herein for the treatment or prophylaxis of a disease or condition associated with deregulation of BET protein activity wherein said disease or condition is independently selected from: cancer; benign proliferative disorders; infectious or non-infectious inflammatory events; autoimmune diseases; inflammatory diseases; systemic inflammatory response syndromes; viral infections and diseases; and ophthalmological conditions.

There is also provided herein PROTAC compounds of formula I in accordance with any aspect herein, for use in the treatment of disease or condition for which a bromodomain inhibitor is indicated and a method of treatment of disease or condition for which a bromodomain inhibitor is indicated by administration of an effective amount of a PROTAC compound of formula I in accordance with any aspect herein, to a mammal, in particular a human in need of such treatment. Examples of disease or condition for which a bromodomain inhibitor is indicated that the compounds of the present invention may be used in the treatment of include diseases associated with systemic inflammatory response syndrome, such as sepsis, burns, pancreatitis, major trauma, haemorrhage and ischaemia.

In such uses or methods the PROTAC compound of Formula I would preferably be administered to a subject in need of such treatment at the point of diagnosis to reduce the incidence of: SIRS, the onset of shock, multi-organ dysfunction syndrome, which includes the onset of acute lung injury, ARDS, acute renal, hepatic, cardiac and gastro-intestinal injury and mortality.

Alternatively in other circumstances where there is a perceived high risk of sepsis, haemorrhage, extensive tissue damage, SIRS or MODS, the PROTAC compound of Formula I would preferably be administered to a subject in need of such protection from such risks, for example prior to surgical or other procedures associated with a high risk of sepsis, haemorrhage, extensive tissue damage, SIRS or MODS.

According to a particular embodiment there is provided herein use of a PROTAC compounds of Formula I for use in the treatment of sepsis, sepsis syndrome, septic shock and/or endotoxaemia.

According to another embodiment there is provided herein use of a PROTAC compounds of Formula I for use in the treatment of acute or chronic pancreatitis, or burns.

Further examples of diseases or conditions for which a bromodomain inhibitor is indicated and for which the PROTAC compounds of Formula I may be used in the treatment of include herpes simplex infections and reactivations, cold sores, herpes zoster infections and reactivations, chickenpox, shingles, human papilloma virus, cervical neoplasia, adenovirus infections, including acute respiratory disease, and poxvirus infections such as cowpox and smallpox and African swine fever virus.

According to further embodiment there is provided herein use of a PROTAC compounds of Formula I for use in the treatment of the treatment of Human papilloma virus infections of skin or cervical epithelia.

In a further aspect there is provided herein, a PROTAC compound of Formula I for use in the treatment of any of the diseases or conditions indicated hereinbefore wherein said treatment modulates one or more of protein methylation, gene expression, cell proliferation, cell differentiation and/or apoptosis in vivo in the disease or condition being treated.

According to a further aspect there is provided a PROTAC compound of Formula I for use in the modulation of one or more one or more of protein methylation, gene expression, cell proliferation, cell differentiation and/or apoptosis in vivo in the treatment of a disease or condition independently selected from: cancer; inflammatory disease; and/or viral disease.

According to another aspect there is provided a therapeutic method of modulating one or more of protein methylation, gene expression, cell proliferation, cell differentiation and/or apoptosis in vivo in the treatment of cancer, inflammatory disease and/or viral disease wherein said method is provided by administering a therapeutically effective amount of one or more PROTAC compounds of Formula I to a subject in need of such therapy.

As also demonstrated herein PROTAC compounds of Formula I, such as for example, compound SIM1, potently and rapidly induce reversible, long-lasting and unexpectedly selective removal of BRD2 over BRD3 and BRD4.

Thus, the invention provides PROTAC compounds of Formula I which bind to a protein within the bromo- and Extra-terminal (BET) family of proteins.

According an aspect the present invention provides PROTAC compounds of Formula I wherein B and D are ligands which bind to a target protein or polypeptide which is to be degraded by ubiquitin ligase, and wherein said target protein is selected from the group consisting of structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity and translation regulator activity.

According an aspect the present invention provides PROTAC compounds having Formula I as defined hereinbefore, wherein B and D are ligands which bind to a target protein or polypeptide which is to be degraded by ubiquitin ligase, and wherein said target protein is selected from the group consisting of B7.1 and B7, TI FRlm, TNFR2, NADPH oxidase, BclIBax and other partners in the apoptosis pathway, C5a receptor, HMG-CoA reductase, PDE V phosphodiesterase type, PDE IV phosphodiesterase type 4, PDE I, PDEII, PDEIII, squalene cyclase inhibitor, CXCRI, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD 124, tyrosine kinase p56 Ick, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus, 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase FIk-IIKDR, vitronectin receptor, integrin receptor, Her-21 neu, telomerase inhibition, cytosolic phospholipaseA2 and EGF receptor tyrosine kinase, ecdysone 20-monooxygenase, ion channel of the GABA gated chloride channel, acetylcholinesterase, voltage-sensitive sodium channel protein, calcium release channel, and chloride channels; acetyl-CoA carboxylase, adenylosuccinate synthetase, protoporphyrinogen oxidase, and enolpyruvylshikimate-phosphate synthase.

According an aspect the present invention provides PROTAC compounds having Formula I, wherein B and D are ligands which bind to a target protein or polypeptide which is to be degraded by ubiquitin ligase, and wherein B or D are an Hsp90 inhibitor; a kinase inhibitor, a phosphatase inhibitor, an MDM2 inhibitor, a compound which targets human BET Bromodomain-containing proteins, an HDAC inhibitor, a human lysine methyltransferase inhibitor, a compound targeting RAF receptor, a compound targeting FKBP, an angiogenesis inhibitor, an immunosuppressive compound, a compound targeting an aryl hydrocarbon receptor, a compound targeting an androgen receptor, a compound targeting an estrogen receptor, a compound targeting a thyroid hormone receptor, a compound targeting HIV protease, a compound targeting HIV integrase, a compound targeting HCV protease or a compound targeting acyl protein thioesterase 1 and/or 2.

According an aspect the present invention provides a method of degrading a target protein in a patient in need comprising administering to said patient an effective amount of a PROTAC compound of Formula I as defined herein, or use of said PROTAC for degrading a target protein in a patient by administration of an effective amount thereof.

According an aspect the present invention provides a method of targeting protein in a cell comprising exposing said cell to an effective amount of a PROTAC compound of Formula I as defined herein, or use of said PROTAC for targeting protein in a cell comprising exposing said cell to an effective amount thereof.

PROTAC compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a PROTAC compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatine capsules comprising the active ingredient together with a) diluents, e.g., lactose, dextrose, sucrose, mannitol, sorbitol, cellulose and/orglycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavours and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a PROTAC compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical compositions of the present invention comprise a therapeutically effective amount of a PROTAC compound of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type.

The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavouring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the PROTAC compounds of the invention with suitable non-irritating excipients or carriers such as cocoa butter, polyethylene glycol or a suppository wax which are solid at ambient temperature but liquid at body temperature and therefore melt in the rectum or vaginal cavity and release the active compound. Solid compositions of a similar type may also be employed as fillers in soft and hard-filled gelatine capsules using such excipients as lactose or milk sugar as well as high molecular weight polyethylene glycols and the like.

The PROTAC compounds can also be provided in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the PROTAC compounds of this invention, excipients such as lactose, talc, silicic acid, aluminium hydroxide, calcium silicates and polyamide powder, or mixtures of these substances. Sprays can additionally contain customary propellants such as chlorofluorohydrocarbons. Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, diseases, conditions, or disorders are treated or prevented in a subject, such as a human or other animal, by administering to the subject a therapeutically effective amount of a PROTAC compound of the invention, in such amounts and for such time as is necessary to achieve the desired result. The term “therapeutically effective amount” of a compound of the invention, as used herein, means a sufficient amount of the compound so as to decrease the symptoms of a disorder in a subject. As is well understood in the medical arts a therapeutically effective amount of a PROTAC compound of this invention will be at a reasonable benefit/risk ratio applicable to any medical treatment.

The dosage for the instant compounds can vary according to many factors, including the type of disease, the age and general condition of the patient, the particular compound administered, and the presence or level of toxicity or adverse effects experienced with the drug. A representative example of a suitable dosage range is from as low as about 0.025 mg to about 1000 mg. However, the dosage administered is generally left to the discretion of the physician.

A wide variety of pharmaceutical dosage forms for mammalian patients can be employed. If a solid dosage is used for oral administration, the preparation can be in the form of a tablet, hard gelatine capsule, troche or lozenge. The amount of solid carrier will vary widely, but generally the amount of the PROTAC compound will be from about 0.025 mg to about 1 g, with the amount of solid carrier making up the difference to the desired tablet, hard gelatine capsule, troche or lozenge size. Thus, the tablet, hard gelatine capsule, troche or lozenge conveniently would have, for example, 0.025 mg, 0.05 mg, 0.1 mg, 0.5 mg, 1 mg, 5 mg, 10 mg, 25 mg, 100 mg, 250 mg, 500 mg, or 1000 mg of the present compound. The tablet, hard gelatine capsule, troche or lozenge is given conveniently once, twice or three times daily.

In general, PROTAC compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors.

In certain embodiments, a therapeutic amount or dose of the compounds of the present invention may range from about 0.1 mg/Kg to about 500 mg/Kg, alternatively from about 1 to about 50 mg/Kg. In general, treatment regimens according to the present invention comprise administration to a patient in need of such treatment from about 10 mg to about 1000 mg of the compound(s) of this invention per day in single or multiple doses. Therapeutic amounts or doses will also vary depending on route of administration, as well as the possibility of co-usage with other agents. Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms. It will be understood, however, that the total daily usage of the compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The invention also provides for pharmaceutical combinations, e.g. a kit, comprising a) a first agent which is a PROTAC compound of the invention as disclosed herein, in free form or in pharmaceutically acceptable salt form, and b) at least one co-agent. The kit can comprise instructions for its administration. The terms “co-administration” or “combined administration” or the like as utilized herein are meant to encompass administration of the selected therapeutic agents to a single patient, and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a PROTAC compound of the invention and a co-agent, are both administered to a patient simultaneously in the form of a single entity or dosage. The term “non-fixed combination” means that the active ingredients, e.g. a PROTAC compound of the invention and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes, oils such as peanut oil, cottonseed oil; safflower oil; sesame oil; olive oil; corn oil and soybean oil; glycols; such a propylene glycol or polyethylene glycol; esters such as ethyl oleate and ethyl laurate, agar; buffering agents such as magnesium hydroxide and aluminium hydroxide; alginic acid; pyrogen-free water, isotonic saline; Ringer's solution; ethyl alcohol, and phosphate buffer solutions, as well as other non-toxic compatible lubricants such as sodium lauryl sulphate and magnesium stearate, as well as colouring agents, releasing agents, coating agents, sweetening, flavouring and perfuming agents, preservatives and antioxidants can also be present in the composition, according to the judgment of the formulator.

### BRIEF DESCRIPTION OF THE FIGURES

FIGS. 1 a) and 1 b) show tertiary complex crystal structures of VHL:MZ1:BRD4^{BD2} (a, PDB:5T35) and BRD4^{BD2}:MT1:BRD4^{BD2} (b, PDB 5JWM) to identify the solvent-exposed region of the compounds for chemical branching of linkers in trivalent PROTAC design. The chemical structures of parent bifunctional molecules MZ1 and MT1 are shown.

FIG. 1 c) shows the chemical structures of designed trivalent PROTACs based on VHL and CRBN E3 ligase ligands.

FIG. 1 d) shows immunoblot analysis of BRD2, BRD3, BRD4 after treatment of HEK293 cells with 1 μM PROTACs or DMSO for 4 h.

FIG. 1 e) shows quantitative live-cell degradation kinetics of CRISPR HiBiT-BRD4 HEK293 cells following treatment with 1 μM compounds or DMSO in quadruplicates. Luminescence (RLU) was continuously monitored in 5 min intervals over a 24 h time period.

FIG. 1 f) is a graph representing the cell viability of MV4; 11 AML cell line following treatment with PROTACs or DMSO for 48 h in in three replicates for each concentration point.

FIG. 2 a) shows Immunoblots of BRD2, BRD3, BRD4 levels in HEK293 cells treated with serially diluted PROTAC compounds SIM1, SIM2 and SIM3 for 4 h. Quantification of BET protein levels was done relative to DMSO control and plots used to measure DC50 values, as shown in FIG. 6d. The blot shown is representative of two independent experiments.

FIG. 2 b) shows quantitative live-cell degradation kinetics of CRISPR HiBiT-BRD2, BRD3, and BRD4 HEK293 cells following treatment with serially diluted SIM1 in quadruplicates. Luminescence (RLU) was continuously monitored over a 24 h time period.

FIG. 2 c) shows calculation of degradation rate, degradation maximum (Dmax), and Dmax₅₀ values from BRD2, BRD3, and BRD4 kinetic profiles shown in panel b.

FIG. 2 d) shows the effects of SIM1 (blue) and cis-SIM1 (red) on the proteome of MV4; 11 cells treated with compound at 10 nM for 4 h. Data plotted log 2 of the fold change versus DMSO control against−log 10 of the P value per protein from three independent experiments. Quantification of representative proteins can be found in FIG. 7d.

FIG. 2 e) shows NanoBRET ubiquitination kinetics of HiBiT-BET proteins following treatment with 10 nM of compound SIM1.

FIG. 3 a) shows a comparison of degradation rate, Dmax, and Dmax₅₀ values calculated from HiBiT-BRD2 kinetic dose response profiles with SIM1 (kinetic profiles show in FIG. 2b), ARV-771 (kinetic profiles shown in FIG. 8a), and MZ1⁴.

FIG. 3 b) shows a graph displaying the quantified expression levels of endogenous BRD2 and Myc in 22Rv1 prostate cancer cell line treated with compounds for 4 h. Curves are a best fit of means from two biologically independent experiments, s.e.m.

FIG. 3 c) shows a graph displaying the loss in CRISPR cMyc-HiBiT protein levels and correlative cell viability in MV4; 11 cells treated with 1 nM concentration of the indicated compounds in quadruplicates. Luminescence and cell viability by CellTiter-Glo were measured at various time points over 24 h.

FIG. 3 d) shows immunoblot of PARP-cleavage in 22Rv1 cells with indicated compounds at 10 nM for 24 h with or without the addition of caspase inhibitor (QVD-OPh, 20 μM) or necroptosis inhibitor (Necrostatin-1, 20 μM). Blots for 48 h treatments and 1 mM MZ1 and MT1 treatments are in FIG. 10.

FIG. 3 e) shows Caspase-Glo 3/7 assays treated with compounds or DMSO for 24 h in 22Rv1 cells. Curves are a best fit of means from three biologically independent experiments, ±s.e.m.

FIG. 3 f) shows images of experiments on the survival of 22Rv1 cells in clonogenic assay. Cells were treated with 10 nM compounds for 24 h. Five hundred cells were re-plated and allowed to grow at 37° C. for 20 days before scanning. Survival fraction was determined by dividing plating efficiency of treated cells by plating efficiency of untreated cells. Error bars indicate the mean values ±S.D. from duplicates.

FIG. 4 a) shows size exclusion chromatography results of complex formation after incubation of SIM1 (red), MZ1 or cis-SIM1 (orange), MT1 (green) or DMSO (cyan) with BD1-BD2 tandem domain from BRD4 (left panel: wild type, middle panel: N140F mutant, right panel: wild type with VCB protein). Intensity of peaks is absorbance at 280 nm.

FIG. 4 b) shows NanoBRET conformational biosensor assay consisting of either the BD1-BD2 tandem domain of BRD4 wild-type (WT), or containing the BD2 N433F mutation flanked by NanoLuc donor and HaloTag acceptor fusion tags. HEK293 cells were transiently transfected with either the WT or N433F mutant biosensor and treated with a serial dilution of compounds SIM1, cis-SIM1, or MT1 compounds in quadruplicates. NanoBRET was measured to determine which treatments showed a conformational change, EC₅₀ s values were calculated and are shown.

FIG. 4 c) shows ITC titrations of BRD4 BD1-BD2 tandem proteins (loaded in the syringe, WT at 200 μM, N-to-F mutants 300 μM) into a 1:1 mixture of SIM1 (16 μM) and VCB (32 μM) pre-incubated into the sample cell.

FIG. 4 d) shows a graph displaying the NanoBRET kinetic ternary complex formation in HEK293 cells transiently expressing HaloTag-VHL paired with either full-length BRD4 WT, N140F or N433F mutants treated with SIM1, cis-SIM1, MT1 or DMSO control in quadruplicates. NanoBRET was continuously monitored for 2 h after compound addition and showed differential levels of ternary complex formation for each BRD4 variant.

FIG. 4 e) shows AlphaLISA titrations of SIM1 in duplicates against biotin-JQ1:BRD4^{BD2} in the absence (red) or presence (blue) of VCB protein.

FIG. 4 f) shows fitted curves from fluorescence polarization competition assays measuring displacement of a FAM-labelled HIF-1alpha peptide from VCB by SIM1 titrated in triplicates, in the presence or absence of BRD4 tandem BD1-BD2.

FIG. 4 g) shows a SPR sensogram to monitor in real-time the interaction of pre-incubated SIM1-BRD4-tandem protein with immobilized biotin-VCB protein. The sensorgram shown is for a ternary single-cycle kinetic (SCK) experiment as representative to three independent experiments, to measure dissociation constant K_{d} and dissociative half-life t_{1/2} of the ternary complex.

FIG. 4 h) shows Live cell compound residence time experiments with BRD4 as measured by NanoBRET target engagement. CRISPR HiBiT-BRD4 cells were incubated with the indicated compounds in quadruplicate followed by addition of a competitive fluorescently-labelled BET inhibitor. NanoBRET was measured kinetically in live cells.

FIG. 5 a) shows the proposed mechanism of SIM1 formation of a 1:1:1 ternary complex with VHL and BET protein. Preferential initial binding of SIM1 to BD2 of BRD4 is followed by conformational change and intramolecular simultaneous binding to BD1. Avidity and cooperativity contribute to formation of a highly stable ternary complex with enhanced residence time at extraordinarily low concentrations of SIM1.

FIG. 5 b) shows different types of PROTAC and molecular glue induced ternary complexes with E3 ligase components and depicted at their varying extents as a factor of PROTAC or degradation concentration. A trivalent complex combining avidity with cooperativity shows the highest and most sustained levels of ternary complex formation, with a minimized hook effect. A cooperative bivalent PROTAC complex is next, followed by a non-cooperative bivalent complex. Lastly a ternary complex induced by molecular glue compounds is shown, which reaches a plateau and unlike PROTACs do not experience the competitive hook effect at higher concentrations.

FIG. 6 (related to FIG. 1) FIG. 6a) shows an inspection of tertiary complex crystal structures BRD4BD1:Bi-BET:BRD4BD1 (PDB: 5ad3) shows the bivalent inhibitor buried inside the protein interface, suggesting derivatization would impair the binding mode.

FIG. 6b) shows quantitative live-cell degradation kinetics of CRISPR HiBiT-BRD4 HEK293 cells stably expressing LgBiT following treatment with serially diluted SIM1-SIM3 in quadruplicates. Luminescence (RLU) was continuously monitored in 5-15 min intervals over a 24 h time period and fractional RLU was determined by normalization to DMSO control.

FIG. 6c) shows the cell viability of A549 lung carcinoma cell line or HL-60 AML cell line following treatment with PROTACs or DMSO for 48 h in three replicates for each concentration point.

FIG. 6d) shows the quantification of each BET protein levels relative to DMSO control of IB data from HEK293 cells of FIG. 2a.

FIG. 7 (related to FIG. 2)

FIGS. 7 a), b) show quantitative live-cell degradation kinetics of CRISPR HiBiT-BRD2, BRD3, and BRD4 HEK293 cells stably expressing LgBiT following treatment with serially diluted SIM2 or SIM3 in quadruplicates. Luminescence (RLU) was continuously monitored in 5-15 min intervals over a 24 h time period and fractional RLU was determined by normalization to DMSO control.

FIG. 7c) shows a calculation of degradation rate, degradation maximum (Dmax), and Dmax50 values for SIM2 (upper set) and SIM3 (lower set) from SIM2 and SIM3 kinetic profiles shown in FIG. 7a or 2b respectively.

FIG. 7d) shows the quantification of representative proteins of mass spectrometry proteomics on FIG. 2d.

FIG. 7e) shows NanoBRET ubiquitination kinetics of HiBiT-BET HEK293 cells expressing LgBiT and HaloTag-Ubiquitin following 100 nM SIM1 or MZ1 or DMSO treatment in quadruplicate. Values are expressed as fold increase over DMSO control.

FIG. 8 (related to FIG. 3)

FIG. 8 a) shows quantitative live-cell degradation kinetics of CRISPR HiBiT-BRD2, BRD3, and BRD4 HEK293 cells following treatment with serially diluted ARV-771 in quadruplicates. Luminescence (RLU) was continuously monitored in 5-15 min intervals over a 24 h time period and fractional RLU was determined by normalization to DMSO control.

FIG. 8b) Comparison of BRD3 and BRD4 degradation rates, degradation maximum (Dmax), and Dmax50 values from kinetic profiles of SIM1 (FIG. 2b), ARV-771 (FIG. 8a), and the previously determined MZ1 (Riching et. al. ACS Chem. Biol. 2018).

FIG. 8c) (upper graph) shows quantified expression levels of endogenous BRD3 and BRD4 in 22Rv1 prostate cancer cell line treated with compounds for 4 h. Curves are a best fit of means from two biologically independent experiments, ±s.e.m. (lower image) Representative images of Western blots for FIG. 3b and upper graph.

FIG. 8d) shows immunoblots of endogenous BRD2, BRD3 and BRD4 in MV4; 11 cells treated in presence of BRD degrader or inhibitor with/without proteasome inhibitor (MG132) or VHL ligand (VH298) for 4 h. Error bars indicate the mean values ±S.D from two biologically independent experiments. SIM1 and cis-SIM1 were used at 100 nM. MG132 was used at 3 μM. VH298 was used at 10 μM.

FIG. 9 (related to FIG. 3c)

FIGS. 9a) and b) show graphs indicating the loss in CRISPR cMyc-HiBiT protein levels (a) and correlative cell viability (b) in MV4; 11 cells treated with 3 nM, 10 nM, 50 nM and 100 nM concentration of the indicated compounds in quadruplicates. Luminescence and cell viability by CellTiter-Glo were measured at various time points over 24 h and normalized to the DMSO control.

FIG. 10 (related to FIGS. 3d,e)

FIG. 10a) shows immunoblot of PARP-cleavage in 22Rv1 cells with SIM1 or cis-SIM1 at 10 nM for the indicated time point with/without the addition of caspase inhibitor (QVD-OPh, 20 μM) or necroptosis inhibitor (Necrostatin-1, 20 μM).

FIG. 10b) shows immunoblot of PARP-cleavage in 22RV1 cells with MT1 at 10 nM or 1 μM for the indicated time point with/without the addition of caspase inhibitor (QVD-OPh, 20 μM) or necroptosis inhibitor (Necrostatin-1, 20 μM).

FIG. 10c) shows immunoblot of PARP-cleavage in 22RV1 cells with MZ1 at 10 nM or 1 μM for the indicated time point with/without the addition of caspase inhibitor (QVD-OPh, 20 μM) or necroptosis inhibitor (Necrostatin-1, 20 μM).

FIG. 10d) shows Caspase-Glo 3/7 assays treated in presence of BET degrader or inhibitor with/without caspase inhibitor (QVD-OPh) or VHL ligand (VH298) for 24 h in 22Rv1 cells. Error bars indicate the mean values ±S.D from three biologically independent experiments. SIM1, cis-SIM1 and MT1 were used at 100 nM. MZ1 and ARV-771 were used at 1 μM. VH298 was used at 10 mM. QvD-OPh was used at 20 μM.

FIG. 11 (related to FIG. 4)

FIG. 11a) shows size exclusion chromatography of complex formation after incubation of SIM1 (red, 25 μM), MZ1 (orange, 25 μM), MT1 (green, 25 μM) or DMSO (cyan, 25 μM) with 25 μM BRD4 BD1-BD2 tandem protein (left panel: N433F mutant, right panel: N140F mutant with VCB protein). Intensity of peaks is absorbance 280 nM.

FIG. 11b) shows NanoBRET target engagement assays in quadruplicate with CRISPR HiBiT-BRD4 HEK293 cells stably expressing LgBiT performed in permeabilized and live cell formats. Cells were treated with a fluorescent BET tracer, then incubated with the indicated compounds at various concentrations to measure competitive displacement. IC50 values for each compound are shown for both permeabilized and live cells.

FIG. 11c) shows NanoBRET ternary complex kinetics of HiBiT-BET HEK293 cells expressing LgBiT and HaloTag-VHL following 100 nM SIM1 or MZ1 or DMSO treatment in quadruplicates. Values are expressed as fold increase over DMSO control.

FIG. 12 shows Table 1 displaying experimental and fitted SPR data for SIM1 binary and ternary complex binding to immobilized VCB.

FIG. 13 shows the chemical structures of SIM1 and its designed negative controls, (R,S)-SIM1 and cis-SIM1. Reversed stereocentres are indicated by asterisks.

FIG. 14 shows BROMOscan displacement titrations by SIM1 and (R,S)-SIM1 from BRD4(1,2). The amount of bromodomain protein measured by qPCR (Signal; y-axis) is plotted against the corresponding compound concentration in log 10 scale (nM, x-axis). Dissociation constants Kd from curve fitting are tabulated. Error values are generated by the GraphPad Prism program and reflect the quality of the fit between the nonlinear least-squares curve and the experimental data.

FIG. 15 shows Immunoblot of degradation of BET proteins in HEK293 cells after treatment with indicated compounds at 1 μM or DMSO for 4 h.

FIG. 16 shows CRISPR HiBiT-BRD2, BRD3, and BRD4 HEK293 cells were treated with 100 nM of DMSO, MZ1, (R,S)-SIM1, and both 10 nM and 100 nM of SIM1 in replicate plates for washout experiments. Media containing the 10 nM and/or 100 nM compounds was removed at 3.5 h, indicated on the graphs, and replaced with media lacking compounds for the remainder of the experiment. Luminescence (RLU) was continuously monitored over a 50 h time period and is plotted normalized to the DMSO control as Fractional RLU.

FIG. 17 shows a bar chart representing the percentage of early (FITC: Apotracker Green) and late (FITC: Apotracker Green and DAPI) apoptotic and healthy MV4; 11 cells after treatment with test compounds atthe indicated concentrations for 24 h, as analysed by Apotracker Green and DAPI staining for viability and surface presence of phosphatidyl serine, respectively, and flow cytometry analysis. Data are plotted as stacked bars so single dots are not shown. Error bars reflect a mean±s.d. of three biological replicates.

FIG. 18 shows mean plasma concentration-time profiles of SIM1 after single intravenous (IV) or subcutaneous (SC) administration (5 mg/kg) to male C57BL/6 mice (n=3). The graph shows that SIM1 exhibits excellent availability and pharmacokinetics exposure in vivo. Further details are in the associated Table 2.

FIG. 19 shows Table 2, which presents calculated pharmacokinetic parameters for intravenous (i.v.) and subcutaneous (s.c.) administration of SIM1 (this work), alongside those of MZ1 (https://opnme.com/molecules/bet-mz-1) and JQ1 (Filippakopoulos et al. Nature 468, 1067-1073 (2010)).

FIG. 20 shows Table 3, which summarises the DC₅₀ values in nM of BRD2, BRD4_long and BRD4_short to quantify the potency of SIM1 and its analogue MN674 at degrading BRD2 and BRD4 isoforms. DC₅₀ values are in nM and are obtained from the dose-dependent curves generated in FIG. 22.

FIG. 21 shows immunoblots demonstrating dose-dependent protein degradation of BRD2 and BRD4 using heterotrivalent PROTACs SIM1 and MN674 in RCC4-HA-VHL.

FIG. 22 shows graphs representing the intensities of the bands of the immunoblots of the dose-dependent protein degradation of BRD2 (FIG. 22a) and BRD4 (FIG. 22b) by MN674 and SIM1 shown on FIG. 21, converted into a percentage band intensity rationalised to the 0.1% DMSO-only control, plotted against the concentration of degrader PROTAC (in nM).

### DETAILED DESCRIPTION

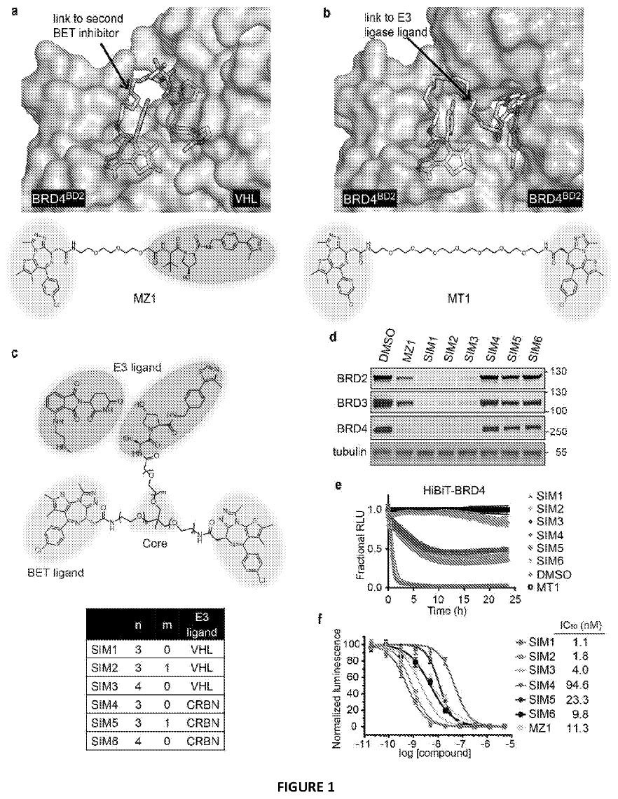

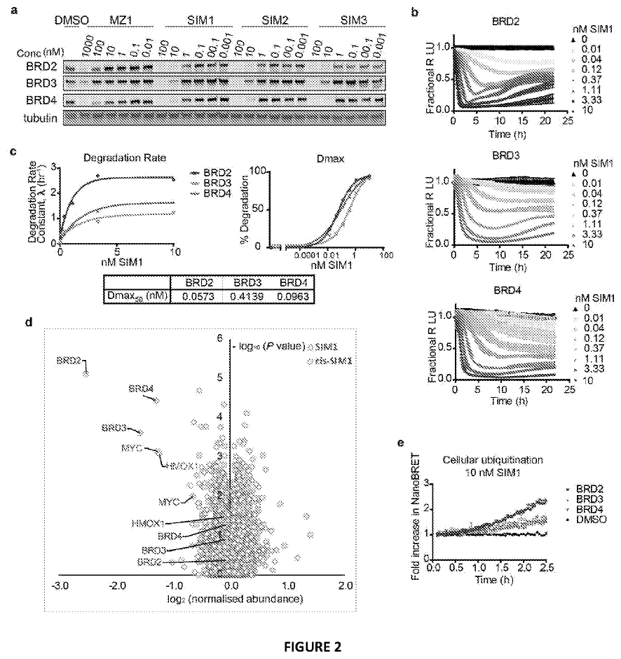

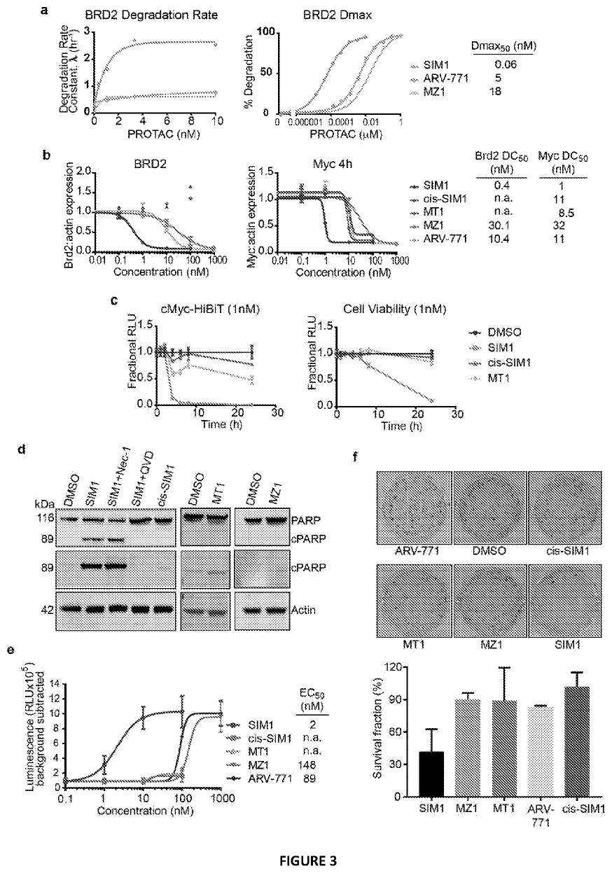

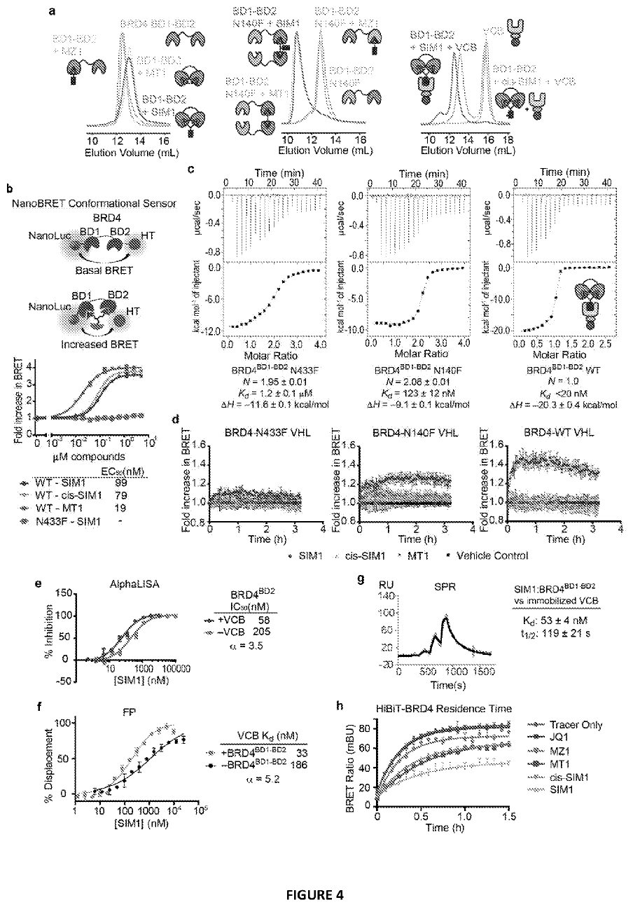

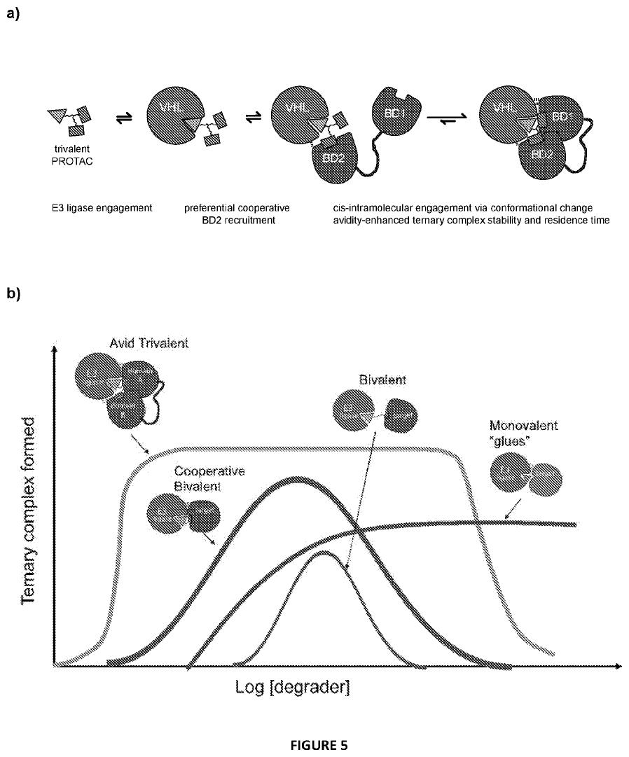

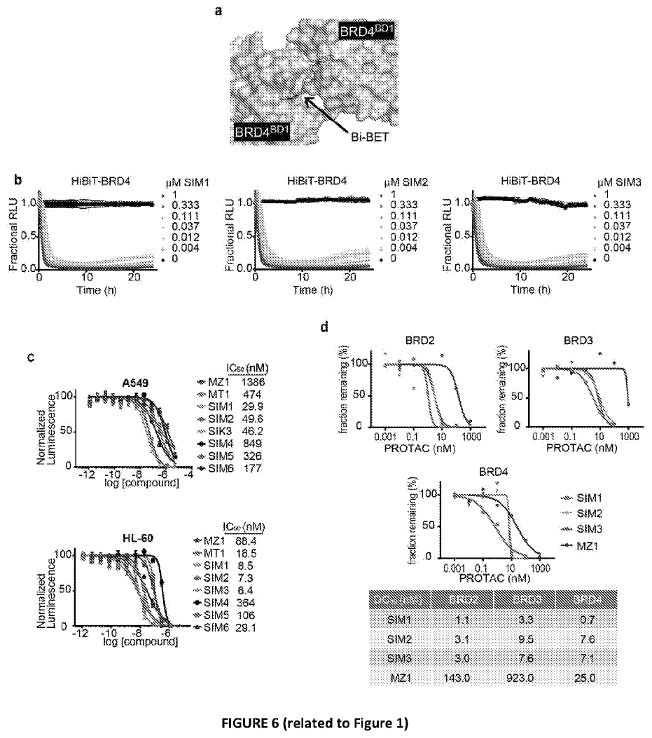

Structure-guided design and synthesis of trivalent PROTACs Several BET family PROTACs, including MZ1 (disclosed in EP3268363), have been developed from parent monovalent BET inhibitors^{9, 11, 37, 38}. The inventors had previously synthesized the potent BET PROTAC compound MZ1,⁹ composed of the VHL ligand VH032 (ref. ³⁹) conjugated to the pan-BET BD inhibitor (+)-JQ1, The crystal structure of MZ1 bound in a ternary complex with VHL and the second bromodomain (BD) of BRD4, BRD4^{BD2, 21} showed that the central portion of the PEG3 linker of MZ1 was solvent exposed, suggesting that the PEG3 linker was a potential a branching point where to link to a second BET ligand (FIG. 1a). Similarly, inspection of the co-crystal structure of the bivalent BET inhibitor MT1, a homodimer of (+)-JQ1 with a PEG7 linker, bound to two monomers of BRD4^{BD2, 40} revealed a site within the central region of the linker for potential chemical branching towards an E3 ligase ligand (FIG. 1b). In contrast, the bivalent inhibitor Bi-BET is fully buried at the interface of the two bromodomains in its co-crystal structure suggesting derivatization could impair its binding mode (FIG. 6a)⁴¹.

The inventors elected MZ1 and MT1 as the progenitor bifunctional molecules in their design strategy. They then devised a suitable ‘core scaffold’ which would enable to assemble three ligands via variable linkers to create trifunctional PROTACs (FIG. 1c). The inventors selected 1,1,1-tris(hydroxymethyl)ethane, also known as trimethylolethane (TME), because it features three primary alcohol groups in a neopentyl core structure, thus acting as branched, close bioisosteric replacements of PEG units. In some embodiments the chemical linkers to each BET ligand moiety were kept identical, resulting in achirality of the central quaternary carbon of the TME. To allow flexibility in exploring the relative constraints between the different monomeric ligands, while at the same time keeping the overall chemical structure as close as possible to those of MZ1 and MT1, we designed three branched PROTACs (SIM1-SIM3) bearing PEG3 or PEG4 at each linker to the BET ligand (n=3,4), and PEG0 or PEG1 towards VH032 (m=0,1) (FIG. 1c). They also designed analogous compounds composed of the CRBN targeting ligand pomalidomide in place of VH032 (SIM4-SIM6) (FIG. 1c).

To synthesize VHL-based trivalent PROTACs of general structure 12 the inventors started from the acetonide 1, synthesized from TME, as key precursor to the trivalent linker core (Scheme 1). Allylation of 1 and subsequent deprotection gave diol 2, which was alkylated with the mesylate linker 3 to give allyl ether 4 as the precursor for PROTACs bearing m=0 to the E3 ligase ligand. To introduce the additional PEG1 unit (m=1), alcohol 1 was reacted with iodide 5 in the presence of sodium hydride. Deprotection of the acetonide group of 6 and subsequent alkylation of the revealed primary alcohols with linker 3 yielded the di-azide 7. Deprotection of the p-methoxybenzyl (PMB) group of 7 with 2,3-Dichloro-5,6-dicyano-1,4-benzoquinone (DDQ) and subsequent allylation afforded allyl ether 8.

Oxidative cleavage of the double bond in allyl ethers 4 or 8 and subsequent Pinnick oxidation of the resulting aldehyde 9 gave carboxylic acid 10, which was coupled with VH032-amine^{27,37}. Reduction of the two azide groups in 11 to amine and further amide coupling with (+)-JQ1 carboxylic acid, completed the preparation of VHL-based trivalent PROTACs. CRBN-based trivalent PROTAC were similarly achieved by coupling an amine-containing analogue of pomalidomide²⁹ in place of VH032-amine.

While the synthesis of cis-SIM1 was straightforward as with its trans diastereomer, initial attempts to synthesize (R,S)-SIM1 compound (see FIG. 13) by using half equivalent of BET ligand at the final coupling step failed to produce the desired 1:1 coupling with an acceptable yield. Therefore, the inventors revised the synthetic route to allow subsequent, independent coupling steps with (+)-JQ1 (Filippakopoulos et al. Nature 468, 1067-1073 (2010)) first, followed by (−)-JQ1 (Filippakopoulos et al. Nature 468, 1067-1073 (2010)) (see Chemistry—Materials and Methods).

SIM1 is a Potent VHL-Based Trivalent PROTAC with Preference for BRD2 Degradation

To evaluate the ability of trivalent compounds to induce intracellular degradation of BET proteins, the inventors first treated human HEK293 cells for 4 hours at 1 μM concentration and assessed levels of BRD2, BRD3 and BRD4 by western blot. Profound degradation across BET proteins was observed with VHL-based SIM1-SIM3, whilst minimal to no-degradation was observed with CRBN-based SIM4-SIM6 (FIG. 1d). To confirm the greater activity of VHL-based compounds compared to CRBN-based PROTACs they next used live cell continual luminescent monitoring of CRISPR/Cas9 endogenously tagged HiBiT-BRD4 in HEK293 cells over 24 h^{29,31}. Faster initial rates of BRD4 degradation accompanied by greater maximal degradation levels were seen with SIM1-SIM3 compared to SIM4-SIM6 (FIG. 1e). Dose response kinetic analysis of HiBiT-BRD4 with SIM1-3 showed rapid and complete degradation from 4 nM-1 μM with no observable hook effect for any of the compounds (FIG. 6b). To confirm greater potency of VHL-based compounds, the inventors evaluated growth inhibition profiles of BET-sensitive cancer cell lines MV4; 11 (FIG. 1f), A549 and HL-60 (FIG. 6c) with compound treatments. Compounds SIM1-SIM3 consistently showed more potent activity relative to SIM4-SIM6, and compared to parent bifunctional molecules MZ1 and MT1 (FIG. 1f and FIG. 6c). Together, the initial screens support greater activity of structure-guided designed VHL degraders over their homologous CRBN molecules.

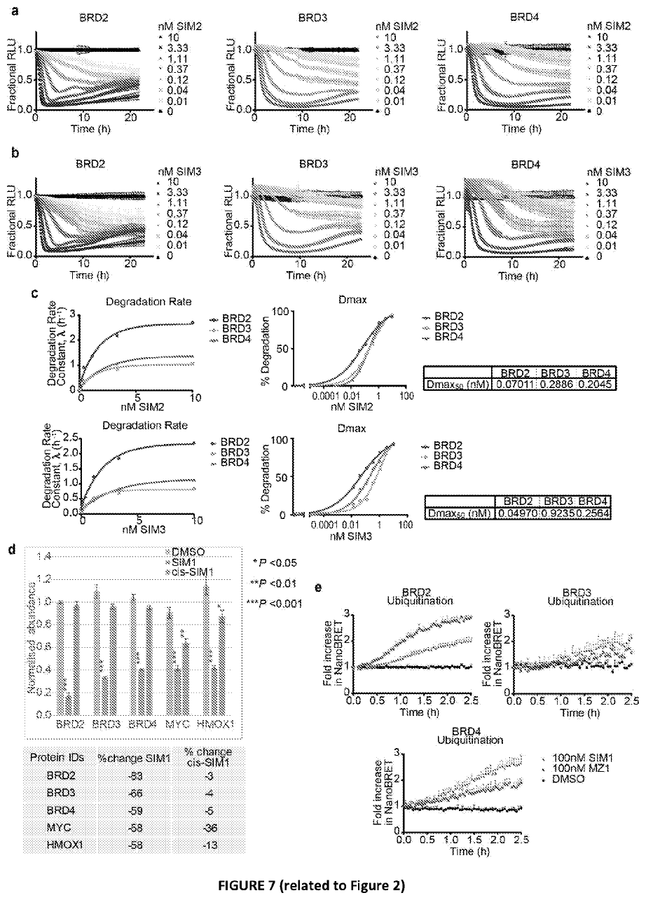

Encouraged by these initial results the inventors next sought to characterize in more depth the degradation activity of the VHL-based degraders across the BET protein family. Concentration-dependent profiling at 4 h treatments using immunoblots evidenced much lower DC₅₀ values of 0.7-9.5 nM for compounds SIM1-SIM3 compared to MZ1 (DC₅₀ values of 25-920 nM) across all the BET proteins, with SIM1 emerging as the most potent of the three degraders (FIG. 2a and FIG. 6d). The inventors also observed a preference for degradation of BRD2 over BRD3 and BRD4 with all compounds, compared to the marked preferential BRD4 degradation of MZ1 (FIG. 6d)^{9,21,25} Quantitative live-cell degradation kinetic assays with compounds SIM1-SIM3 were performed with CRISPR HiBiT cell lines of BET family members BRD2, 3, and 4 and compounds SIM1-SIM3 titrations up to 10 nM concentrations (FIG. 2b and FIG. 7a,b). These analyses further qualified compound SIM1 as the most potent degrader across all BET family members (Dmax₅₀ 60-400 μM) (FIG. 2b,c and FIG. 7a-c), with BRD2 showing the fastest degradation rate and lowest Dmax₅₀ values across all compounds (FIG. 2c and FIG. 7c).

(R,S)-SIM1 was also tested for degradation activity and was found to behave similarly to MZ1 and less potently than SIM1 for degradation of all BET family members (see FIG. 15, which shows an immunoblot of the degradation of BET proteins in HEK293 cells after treatment with indicated compounds at 1 μM or DMSO for 4 h).

To assess whether the trivalent PROTAC induced also a more sustained degradation of BET proteins in cells compared to MZ1 or (R,S)-SIM1, degradation washout experiments were performed. CRISPR HiBiT-BET HEK293 cells were treated with equivalent concentration (100 nM) of SIM1, (R,S)-SIM1 and MZ1 compounds for 3.5 h, then media was removed and replaced with media lacking compounds. The inventors also tested SIM1 at 10× lower concentration (10 nM) to account for its higher potency of degradation across BET proteins. The HiBiT-BET protein levels were continuously monitored from the initial addition of the compounds and immediately after the wash for a total time of 50 h. At the 100 nM treatment, degradation of all BET family members by SIM1 remained at constant low levels after washout over the time course, while at the 10 nM SIM1 treatment partial recovery was observed for BRD2, BRD3, and BRD4 after washout (FIG. 16). Recovery of all BET family members following washout for cells treated with 100 nM (R,S)-SIM1 or 100 nM MZ1 was greater and occurred faster than those observed with SIM1 (FIG. 16).

To assess the cellular selectivity of compound SIM1 for BET proteins, multiplexed tandem mass tag (TMT) labelling mass spectrometry proteomic experiments were performed to monitor protein levels in a quantitative and unbiased fashion. To furnish a suitable non-degrading negative control isomer in these experiments, the inventors synthesized compound cis-SIM1 bearing inverted stereochemistry at the hydroxyproline centre of the VHL ligand, resulting in loss of binding to VHL (see Chemistry—Materials and Methods)^{9,10} Acute myeloid leukaemia (AML) Mv4; 11 cells were treated in triplicate with DMSO, 10 nM of compound SIM1, or 10 nM of compound cis-SIM1 for 4 h. Among the 5,232 proteins quantified, BRD2 was found as most significantly degraded by compound SIM1 compared to compound cis-SIM1, followed by BRD3 and BRD4 (FIG. 2d and FIG. 7d).

The inventors also observed a slight decrease in protein levels for MYC, which is a known downstream effect of BET-induced degradation, and HMOX1 (heme oxygenase 1) suggesting early initiation of apoptosis⁴².

As enhanced rate and/or levels of degradation have been found to correlate with enhanced ubiquitination, cellular studies were performed to monitor the kinetics of ubiquitination of BET family members using bioluminescence resonance energy transfer (NanoBRET) assays consisting of the HiBiT-BET CRISPR cell lines expressing fluorescently labelled HaloTag-Ubiquitin³¹. Shown in FIG. 2e, the kinetic increase in cellular ubiquitination was more robust for BRD2 as compared to BRD3 and BRD4 after a treatment with 10 nM of compound SIM1.

These same trends were observed at a concentration of 100 nM of compound SIM1. Comparison with MZ1 revealed that compound SIM1 led to higher levels of ubiquitination of all BET family members, with the greatest difference observed for BRD2 (FIG. 7e). Together, these cellular degradation data highlighted compound SIM1 as the most potent BET degrader amongst the studied trivalent PROTACs, with preferential degradation of BRD2.

Compound SIM1 is Functionally More Potent than Bivalent BET PROTACs and Parent Bifunctional Inhibitors

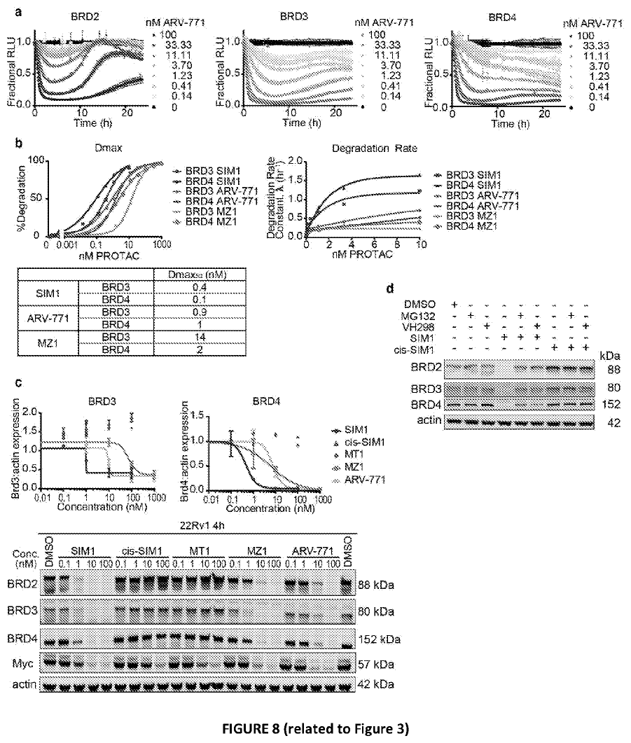

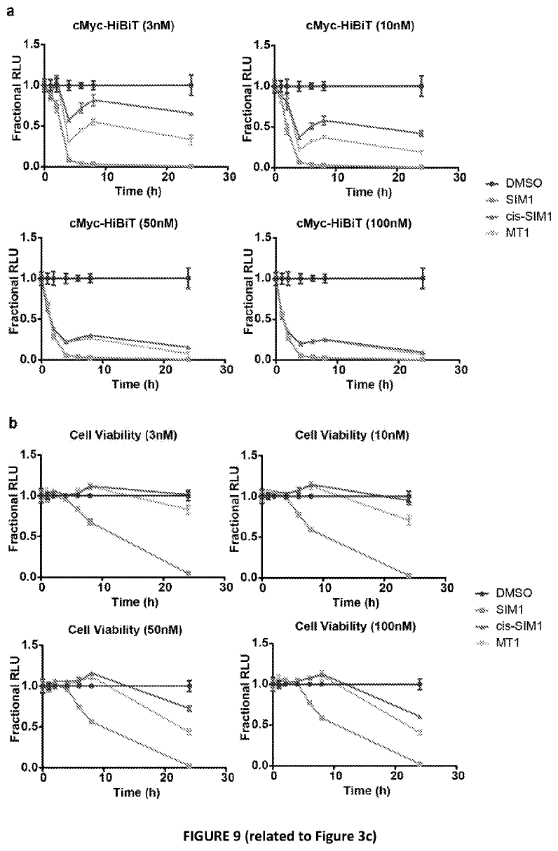

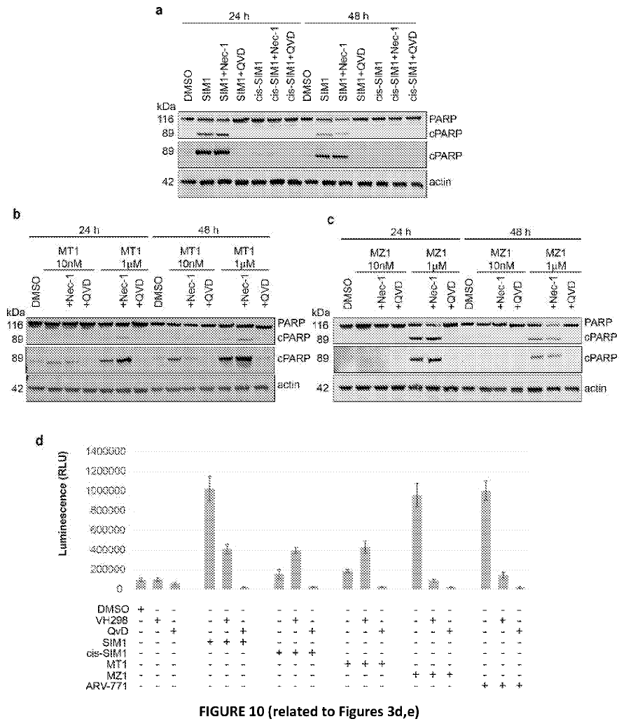

To determine whether increase in target binding valency of compound SIM1 improved both degradation and efficacy as compared to other VHL-based bivalent PROTACs, kinetic degradation profiles for each BET family members were first determined for the bivalent BET PROTAC ARV-771³⁷ (FIG. 8a). Calculated from these and the previously published MZ1 analysis³¹, degradation rates and Dmax₅₀ values as compared to compound SIM1 were plotted for BRD2 (FIG. 3a), as well as BRD3 and BRD4 (FIG. 8b). These quantitative comparisons revealed that compound SIM1 had a vastly improved rate of degradation as well as Dmax₅₀ values ranging from increases in degradation potency of 80-300× for BRD2 (FIG. 3a), 2-35× for BRD3, and 10-20× for BRD4 (FIG. 8b).

To understand if the improvement in degradation of the trivalent PROTAC relative to bivalent BET PROTACs translated to enhanced functional outcomes, the inventors next moved to study the response of compound SIM1 in a BET-sensitive cell line, the prostate cancer line 22Rv1. Treatment of 22Rv1 cells with varying concentrations of compounds at the early time point of 4 h confirmed the enhancement in BET degradation potency and cMyc level suppression activity of compound SIM1 compared to related bifunctional BET degraders MZ1 and ARV-771, as well as non-degrading controls MT1 and cis-SIM1 (FIG. 3b and FIG. 8c).

The expected mechanistic dependency of compound SIM1-induced degradation on functional VHL E3 ligase and proteasomal activity was confirmed with co-treatment with VHL inhibitor VH298³⁹ and proteasome inhibitor MG132 (FIG. 8d).

BET inhibitors and degraders result in down-regulation of numerous targets, including cMyc, which showed loss after 4 h treatment with 10 nM of compound SIM1 in unbiased proteomics profiling in the BET-relevant AML line of MV4; 11 (FIG. 2d). To quantitate the time-dependent loss of cMyc in this cancer cell line, cMyc was endogenously tagged with HiBiT and protein levels were monitored in cell lysates at varying times with different concentrations of compounds SIM1, cis-SIM1, and MT1 (FIG. 3c, left and FIG. 9a). Rapid and complete loss of cMyc was observed with 1 nM SIM1 concentration (FIG. 3c, left) whereas similar levels of cMyc loss with the parent MT1 or cis-SIM1 were not achieved until 50-100 nM treatments (FIG. 9a). Correlative cell viability assays were performed to understand how each compound impacted cell death, and these revealed that only treatment with SIM1 at 1 nM (and not cis-SIM1 or MT1) resulted in measurable cellular death after 6 h (FIG. 3c, right). Similar trends were observed at higher compounds concentrations where SIM1 was found to be significantly more cytotoxic than control compounds at any concentration after 6 h (FIG. 9b).

Next, the inventors assessed the functional effects of SIM1 on the viability of 22Rv1 cancer cells. Substantial cell death was observed after 24 h treatment with 10 nM SIM1, as shown by PARP cleavage assays (FIG. 3d and FIG. 10a). In contrast, MT1 or MZ1 did not cause observable PARP cleavage even up to 48 h treatment at the same concentration as SIM1 (FIG. 10b-c). Similar levels of PARP cleavage were observed with MT1 and MZ1 only at 1 μM concentration (FIG. 10b-c). Cells died of apoptosis as indicated by prevented cleavage of PARP upon co-treatment with QVD-OPh, a pan-caspase inhibitor, and not of necroptosis, as shown by co-treatment with Necrostatin-1 (FIG. 3d and FIG. 10a).

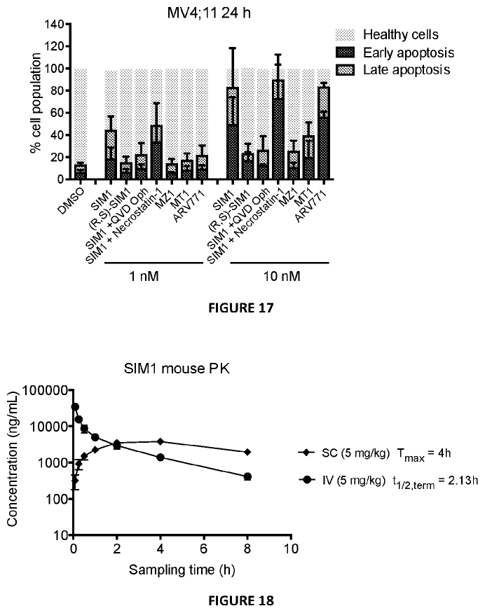

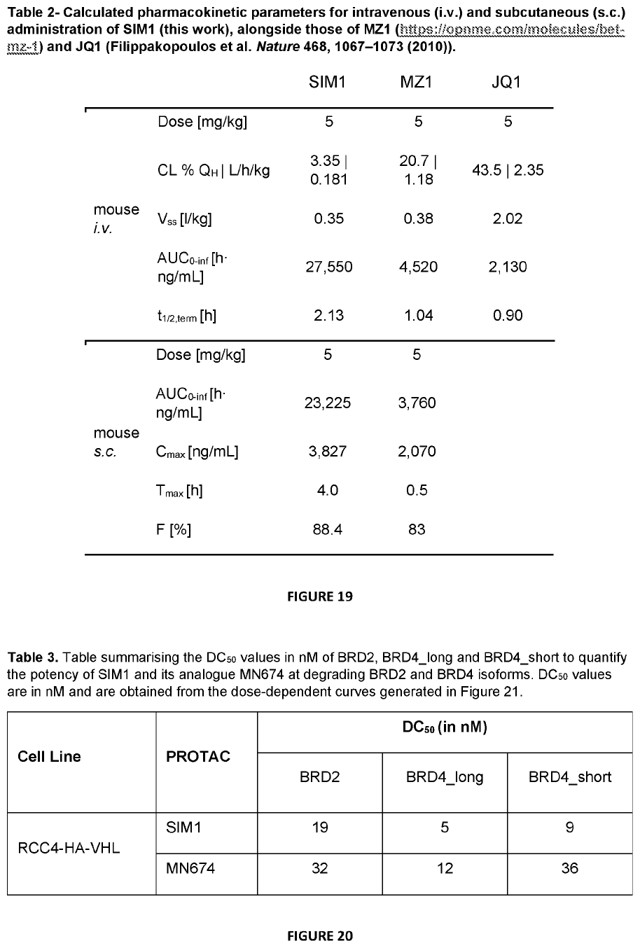

Early and late apoptotic induction between compound treatment was compared and notably, SIM1 induced a much greater degree of both early and late apoptosis at 1 nM compared to all bivalent counterparts tested, even when compared to tenfold higher concentration of (R,S)-SIM1, MZ1, or MT1 (see FIG. 17, which shows the percentage of early (FITC: Apotracker Green) and late (FITC: Apotracker Green and DAPI) apoptotic and healthy MV4; 11 cells after treatment with test compounds at the indicated concentrations for 24 h, as analysed by Apotracker Green and DAPI staining for viability and surface presence of phosphatidyl serine, respectively, and flow cytometry analysis. Data are plotted as stacked bars so single dots are not shown. Error bars reflect a mean±s.d. of three biological replicates). Together, the biological data supports more potent degradation and more substantial downstream functional activity of the trivalent degrader SIM1 compared to (R,S)-SIM1 and parent bivalent degraders or inhibitors.

Caspase-Glo assays confirmed the significantly more potent activity of compound SIM1 (EC₅₀ 2 nM) compared to bifunctional degraders MZ1 and ARV-771 (EC₅₀ 150 and 90 nM, respectively), with much greater maximal signal than non-degraders cis-SIM1 and MT1 (FIG. 3e). Caspase-Glo activity of compounds SIM1, MZ1, and ARV-771 was blocked by co-treatment with the VH298 and Q-VD-OPh, whereas only Q-VD-OPh and not VH298 co-treatment blocked activity of cis-SIM1 and MT1, consistent with their different mode of action (FIG. 10d).

The superior activity of compound SIM1 was evidenced in a colony-formation assay where 22Rv1 cells were treated with test compounds at 10 nM concentration for 24 h, and treated cells re-plated and then allowed to grow for 20 days. In this assay, only treatment with compound SIM1 resulted in significant cytotoxicity compared to vehicle control (FIG. 3f). Together, the biological data supports more potent degradation and more substantial downstream functional activity of the trivalent degrader compound SIM1 compared to benchmark parent bivalent degraders or inhibitors.

Finally, the inventors evaluated the pharmacokinetics (PK) of SIM1 following intravenous and subcutaneous administration in mice (see FIG. 18). SIM1 exhibited highly favourable bioavailability and stability, including high AUCs, low clearance and long half-lives, comparing positively to those of the more canonical small-molecule components i.e. monovalent JQ1 36 and bivalent MZ1 (data available on OpnMe.com) (FIG. 18 and Table 2). Such favourable PK profile is remarkable given its large size (molecular weight 1,619 Da) and qualifies SIM1 as chemical probe appropriate for in vivo use.

Compound SIM1 Engages BD1 and BD2 Intramolecularly and Forms a 1:1:1 Ternary Complex with VHL and BRD4

The remarkable cellular activity and potency of compound SIM1 prompted the inventors to interrogate the molecular recognition and stoichiometry of trivalent PROTAC complexes with VHL and BRD4 as representative BET protein. Previous work had established that bivalent BET inhibitors intramolecularly and simultaneously engage BD1 and BD2 bromodomains within a BET protein^{40,41}.

The inventors thus hypothesized that trivalent BET PROTAC SIM1 could also display cis intramolecular engagement to underpin its mechanism of action. To address this, they first employed biophysical binding assays with recombinant proteins. They performed size-exclusion chromatography (SEC)²⁷ experiments using tandem BD1-BD2 constructs from BRD4 that were either wild-type (WT), so competent to cis intramolecular binding, or contained a point mutation in either BD1 or BD2 at a conserved asparagine residue in the ligand-binding pocket (N140F in BRD4^{BD1}, or N433F in BRD4^{BD2}) to abrogate binding to one of the two bromodomains thus rendering the mutant tandem unable to engage in a cis bivalent fashion⁴¹.

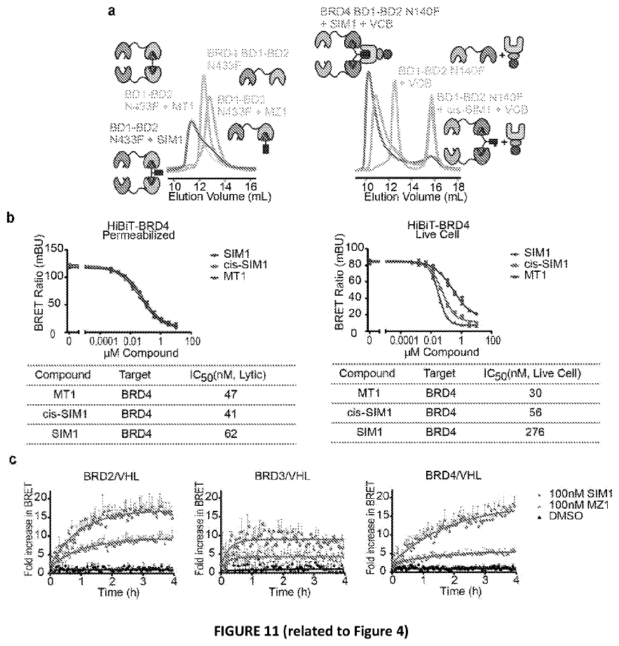

Compound SIM1 was able to shift the SEC profile of BRD4 wild-type BD1-BD2 tandem construct to a higher elution volume, compared to free or MZ1-bound BRD4, consistent with the formation of a more compact intramolecular 1:1 complex with compound SIM1, as observed with MT1 (FIG. 4a). In contrast, when either N140F or N433F mutant tandems were used, we observed a significant shift to much lower elution volumes with both compounds SIM1 and MT1, consistent with the formation of the higher-molecular weight dimer species in solution, compared to the reference free or MZ1-bound BRD4, consistent with the formation of higher-molecular weight 2:1 species in solution (FIG. 4a and FIG. 11a).

Having established that compound SIM1 engages BD1 and BD2 in a cis intramolecular fashion, using its bivalent BET ligand portion, the inventors next asked whether compound SIM1 could then form the expected 1:1:1 complex between the BD1-BD2 tandem domain and the E3 ligase VHL, by utilizing the remaining binding valency from the VHL ligand portion. Indeed, a sample containing 1:1:1 equivalents of compound SIM1, BD1-BD2, and VHL-ElonginB-ElonginC complex (VCB) ran as a single species and at lower elution volumes compared to either of the two peaks observed from a sample containing the same equivalent ratio of cis-SIM1, BD1-BD2 tandem and VCB, where only the 1:1 cis-SIM1:BD1-BD2 complex and unbound VCB can be formed (FIG. 4a).

Previous biophysical and cellular studies with bivalent BET inhibitors, showed that the intramolecular binding of BD1 and BD2 resulted in structural conformational change of BRD4⁴¹. To determine if compound SIM1 cellular binding to BRD4 could also induce a structural change, the inventors utilized a NanoBRET biosensor containing the same BD1-BD2 tandem domains of wild-type BRD4 or mutant N433F, flanked respectively by a NanoLuc donor and HaloTag acceptor (FIG. 4b). With the BD1-BD2 tandem WT sensor, all compounds showed a change in BRD4 conformation, manifested by an increase in NanoBRET signal which reached and maintained a plateau, as is expected for a intramolecular engagement (FIG. 4b). Given the similar patterns of cis-SIM1 and SIM1, the ability to engage VHL did not appear to significantly impact the structural change in BRD4 (FIG. 4b). As a control, the BD1-BD2 N433F mutant sensor showed no response indicating that simultaneous binding of BD1 and BD2 is required for the conformation change (FIG. 4b). Interestingly cis-SIM1 and SIM1 showed higher EC₅₀ values for BRD4 engagement than MT1 (FIG. 4b).

To determine if this is due to a reduced binding affinity of BRD4 and/or reduced permeability given their increase in molecular weight, NanoBRET target engagement assays were performed measuring displacement of a fluorescent BET tracer molecule bound to HiBiT-BRD4⁴¹. In permeabilized cells, where permeability is not a factor, the inventors observed binding of SIM1, cis-SIM1 and MT1 to endogenous HiBiT-BRD4 with near-identical binding affinities and IC₅₀ values (FIG. 11b). However in live cells, both SIM1 and cis-SIM1 showed reduced binding affinity to BRD4 compared to MT1, with the same 10× shift observed in the conformational sensor assay, suggesting this reflects reduced permeability of the trivalent molecule relative to the parent bivalent inhibitor (FIG. 11b).

Despite this reduced cellular permeability, compound SIM1 is a highly potent degrader and these biochemical and cellular studies give insights as to its mode of ternary complex formation, wherein compound SIM1 simultaneously engages BD1 and BD2 within a given BET protein, and recruits VHL to form a 1:1:1 complex.

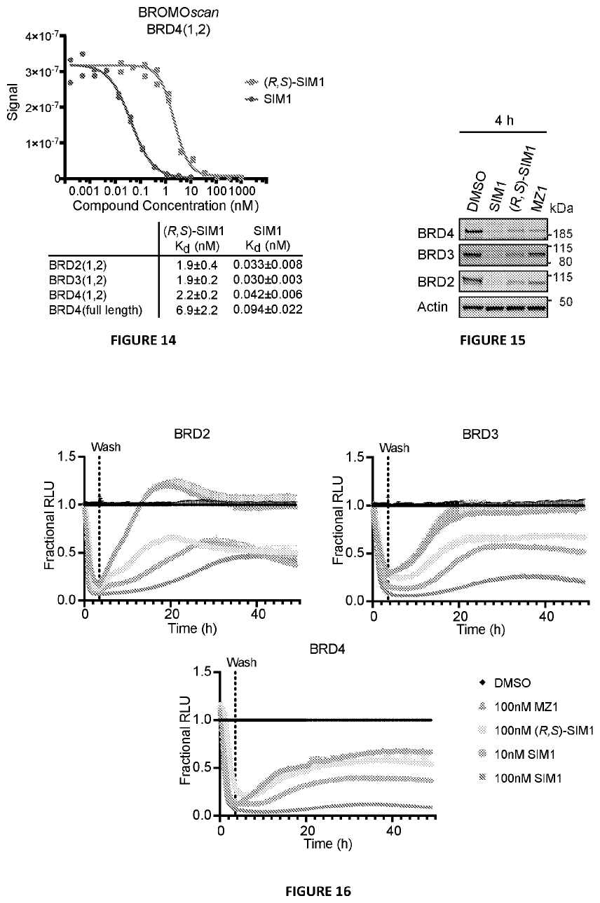

(R,S)-SIM1, was used it as monodentate BET binder control to ask to what extent the bidentate BET binder SIM1 might exhibit avidity, i.e. enhanced binding affinity for BET proteins due to intramolecular BD1 and BD2 binding. We used established phage-based bromodomain displacement assays to quantitatively measure compound binding with tandem bromodomain constructs. Bidentate SIM1 showed picomolar affinity to tandem bromodomain constructs BRD2(1,2), BRD3(1,2), BRD4(1,2) and full-length BRD4, with 50-90× increase in affinity compared to monodentate (R,S)-SIM1 evidencing its avidity (see FIG. 14).

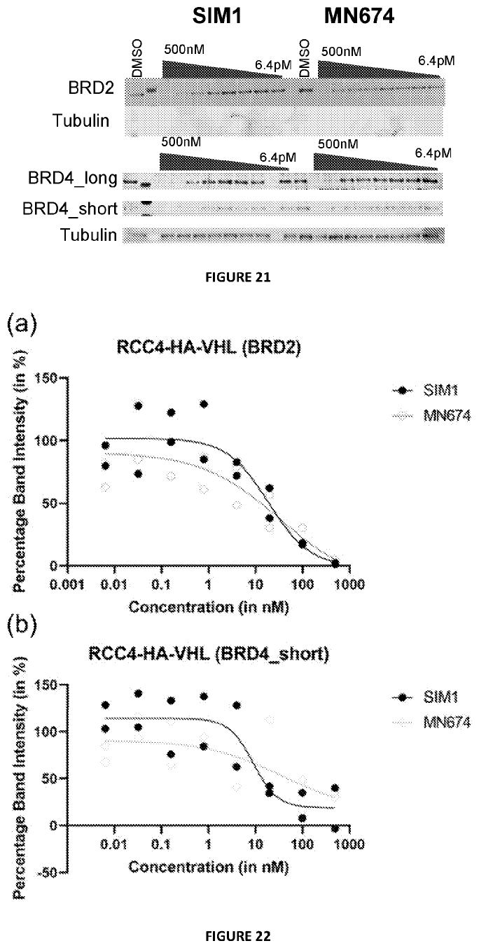

Compound MN674 was designed and synthesised as another analogue of SIM1. The structure of MN674 is the same as for SIM1, but contains one less PEG unit in the linkers to the JQ1 binders. one less PEG unit in the linkers to the JQ1 binders. MN674 was tested in the mammalian cell line RCC4-HA-VHL in a dose-response degradation assay. The assay involved immunoblots (see FIG. 21 for raw western blot data and FIG. 22 for dose-response curves) displaying 6 hour treatments of eight 5-fold serial dilutions from 500 nM to 6.4 pM of each compound. Quantification of protein levels of BRD2 and BRD4 were performed to measure DC50s values, that are tabulated in Table 3 (FIG. 20). FIG. 21 shows the results of immunoblots demonstrating dose-dependent protein degradation of Brd2 and Brd4 using heterotrivalent PROTACs SIM1 and MN674 in RCC4-HA-VHL. RCC4-HA-VHL cells were exposed to eight 5-fold serial dilutions (from 500 nM to 6.4 μM) of heterotrivalent Bet-Bet-VHL PROTACs SIM1 and MN674 for 6 hour treatments, before lysates were harvested and analysed on immunoblots (FIG. 21). A Bio-Rad MP imaging system was used for fluorescence detection of BRD2 (IRDye 800CW), BRD4_long (IRDye 800CW), BRD4_short (IRDye 800CW) and house-keeping gene Tubulin (Rhodamine). The intensities of the bands were determined using ImageLab software and all BET protein intensities normalised to Tubulin in order to convert them into a percentage band intensity rationalised to the 0.1% DMSO-only control. These intensities were then plotted in GraphPad Prism for Dose-Response [Inhibition] Variable slope (four parameter) analyses to generate the curves for BRD2 (FIG. 22a) and BRD4 (FIG. 22b), to determine the DC50 values presented in Table 3 (FIG. 20). The experiment is representative of n=2 i.e. biological duplicate.

Overall, MN674 was shown to be comparable in degradation potencies to SIM1, albeit with slightly differential outcome depending on the different cell lines.

Compound SIM1 Forms Cooperative Stable Ternary Complexes with Enhanced Cellular Residence Time

To validate the ternary complex stoichiometry and further characterize the binding thermodynamics and kinetics of their formation and dissociation, the inventors next used isothermal titration calorimetry (ITC) by performing reverse titrations^{21,32} First, in titrations of BRD4 N140F or N433F tandems into preformed SIM1:VCB complex we observed 2:1 stoichiometry, molar binding enthalpy of ΔH=−9.1 and −11.6 kcal/mol, and K_{d}=0.12 and 1.2 μM, respectively. In contrast, titration of BRD4 WT BD1-BD2 under identical conditions displayed binding stoichiometry of 1:1, a large negative binding enthalpy (ΔH=−20 kcal/mol), and a much lower dissociation constant (K_{d}<20 nM) (FIG. 4c). Together, the ITC data confirmed the stoichiometries observed in SEC and point to preferential engagement of BRD4-BD2 by VCB:SIM1 as likely first step prior to intramolecular 1:1 engagement.

To study the favourability of engaging both BD1 and BD2 in ternary complex with VHL in the context of cellular, full-length BRD4, the inventors interrogated VHL binding to full-length BRD4 WT, N140F, or N433F mutations using kinetic NanoBRET ternary complex assays³¹. HEK293 cells expressing the various NanoLuc-BRD4 fusions with HaloTag-VHL showed ternary complex with VHL and WT BRD4 was rapidly formed in the presence of SIM1, but not with controls cis-SIM1 or MT1 (FIG. 4d). Ternary complex formation however was markedly reduced with the BRD4 N140F mutation and almost abolished with the N433F mutation (FIG. 4d). These results confirm that compound SIM1 utilizes both BD1 and BD2 for ternary complex formation and suggest preferential binding to BD2 over BD1, consistent with the ITC results and what was found previously with MZ1^{21,32}.

Cellular NanoBRET ternary complex formation with compound SIM1 compared to MZ1 was also assessed with the panel of endogenously tagged HiBiT BET family members and a more robust and sustained ternary complex was observed for BRD2 and BRD4 with VHL induced by compound SIM1 as compared to MZ1, while that with BRD3 did not appear to be as prolonged or stable (FIG. 11c), again similar to what was found previously with MZ1^{31,32}.

The inventors next examined ternary complex cooperativity demonstrating that compound SIM1 exhibited a positive cooperativity α value of 3.5 for ternary complex formation as shown in competitive AlphaLISA assays (FIG. 4e), measuring binding of compound SIM1 alone (IC₅₀=205 nM) or SIM1:VCB binary complex (IC₅₀=58 nM) via the displacement of biotinylated JQ1 (ref. ⁴³) from BRD4^{BD2}. As a cross-validation, the inventors evaluated cooperativity in a competitive FP assay measuring binding at the VHL end of the PROTAC molecule via the displacement of a fluorescent HIF-1α peptide probe^{11,32} Again, in this experiment compound SIM1 exhibited positive cooperativity (α=5.2) from enhanced affinity of its competitive displacement as binary SIM1:BD1-BD2 tandem (K_{d}=33 nM) compared to SIM1 alone (K_{d}=186 nM) (FIG. 4f).

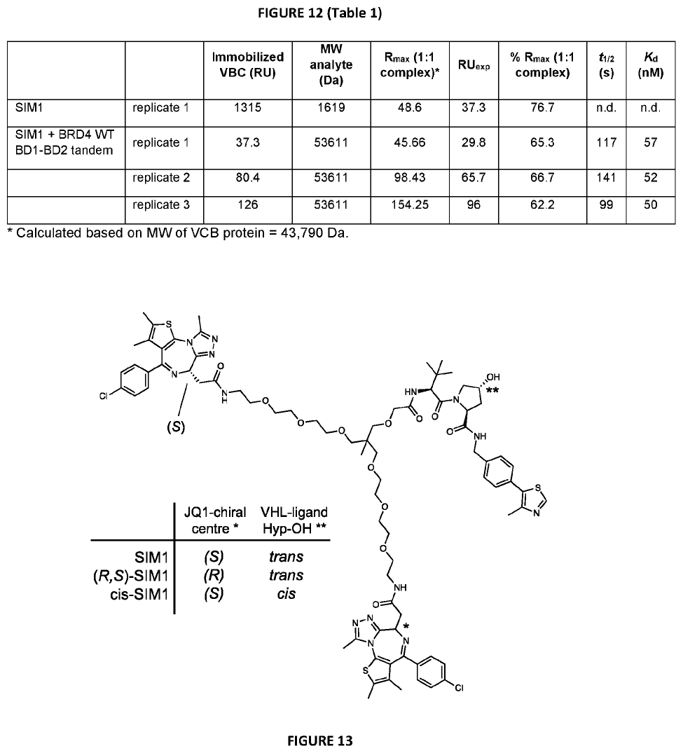

Finally, the inventors assessed formation of ternary complexes of compound SIM1 in an SPR binding assay as previously described³². They immobilized biotinylated VCB onto the surface chip and injected serially-diluted solution of compound SIM1 pre-incubated in excess of BD1-BD2 protein, in a single-cycle kinetic format experiment (FIG. 4g)³². SIM1:BD1-BD2 bound to VCB in 1:1 stoichiometry, as evidenced by the experimental % Rmax value for the expected 1:1 binding model (64.7±2.3%) being comparable to and not exceeding that observed from a titration of SIM1 alone (77%) (FIG. 12—Table 1). Compound SIM1 formed a high-affinity, stable and long-lived 1:1:1 complex with wild-type BD1-BD2 and VCB (t_{1/2}=119±21 s; K_{d}=53±4 nM) (FIG. 4g and FIG. 12—Table 1).

The positive cooperativity of compound SIM1 as well as previous studies showing bivalent inhibitors exhibit longer target residence time as a mechanism for improved efficacy compared to their monovalent counterparts prompted us to explore whether the cis-binding mode of compound SIM1 also contributed to any changes in residence time, either to the target or within the ternary complex in live cells.

To monitor residence time, the HiBiT-BRD4 CRISPR cells were first incubated with saturating concentrations of PROTAC or inhibitor compounds, followed by the competitive BET fluorescent tracer. The NanoBRET signal produced from this displacement can be monitored kinetically in live cells, the rate and intensity of which directly correlates to the to the residence time of the initial compound-bound complex⁴¹. In agreement with previous studies, JQ1 had a short residence time⁴¹, while MT1 showed longer residence time, as expected for a bivalent inhibitor (FIG. 4h). Interestingly MZ1 showed a residence time between the monovalent and bivalent BET inhibitors, indicating even with monovalent BET target engagement, improvements in residence time are possible with a PROTAC (FIG. 4h), which the inventors ascribe to its cooperative binding²¹. Strikingly, differences were observed between compounds cis-SIM1 and SIM1, with SIM1 showing the longest residence time on BRD4 while cis-SIM1 showed identical displacement kinetics as MT1 (FIG. 4h).

The different behaviours between cis-SIM1, (R,S)-SIM1 and SIM1, all of which exhibit avidity from bivalent target engagement, indicate that recruitment of VHL by SIM1 significantly increases the residence time on BRD4, due to the formation of a highly stable and cooperative ternary complex inside the cell. Together, the results from these biophysical binding assays and cellular studies in vitro demonstrate that compound SIM1 forms cooperative, stable and long-lived 1:1:1 ternary complex with VHL and BET proteins. Furthermore, the inventor's findings suggest that this is facilitated by intramolecular binding that results in both a structural change as well as prolonged compound interaction with the target, which is even further enhanced by the ability to engage VHL in the ternary complex.

### Discussion of Results

The inventors have demonstrated that the novel trivalent PROTACs of the invention SIM1-SIM6, and in particular SIM1 are an exquisitely potent and profoundly active degrader of BET proteins. The inventor's biological and mechanistic investigation of compound SIM1 provides proof-of-concept for augmenting the valency of PROTAC degraders as a viable strategy to boost their mode of action by positively impacting the ternary complex. To understand the mechanism of action of compound SIM1, a series of biophysical, biochemical, and cellular studies were performed. Most critically these showed a 1:1:1 BRD4:SIM1:VHL complex stoichiometry and SIM1 bound intramolecularly in a cis-fashion to both BD1 and BD2 of BRD4, inducing a conformational change (FIG. 5a). Further investigation with BRD4 BD1 or BD2 mutants suggested there is sequential or preferred BD binding of SIM1 into BRD4, with BD2 being the more critical first step (FIG. 5a), consistent with the BD2-specificity of the parent PROTAC MZ1²¹,32. Based upon the extensive data presented here and degradation patterns of all BET family members, the inventors thus propose all BET family engage with compound SIM1 in this same fashion.

Interestingly, BRD2 was found in a series of orthogonal assays to show the highest level of degradation and correspondingly the most robust level of ubiquitination of the family members, which is unprecedented preference for BRD2 from this class of BET PROTAC degraders. It is not known why there is this preference, but it could reflect greater avidity and ternary complex stability of BRD2 with VHL, or the possibility that the induced structural change better positions BRD2 in a more favourable state for more productive ubiquitination, as compared to BRD3 or BRD4.

For successful degradation to occur via PROTACs, the induced ternary complex between the target protein and the E3 ligase not only must form, but the target within the complex must also be well positioned for efficient ubiquitination. To achieve this structural favourability, numerous linkers are tested in the development of PROTACs to discover compounds which optimally recruit the target in a position and geometry productive for E2/E3 catalysed neo-substrate ubiquitination^{21, 34, 44}. An additional factor which can be a crucial factor to success, perhaps even able to overcome non-optimal structural positioning, is kinetic and thermodynamic favourability of ternary complex formation. In these cases, PROTACs facilitate positive neo-interactions between the E3 ligase and target, similar to the mechanism of monovalent molecular glues, resulting in stable, cooperative, and long-lived ternary complexes which drive catalytic ubiquitination^{21, 31, 32, 45}. For compounds which do not have this latter ternary complex favourability, their window of degradation efficacy will be limited by the hook effect as the binary complexes will have higher preference of formation than the ternary complex (FIG. 5b)^{17, 46}. On the other hand, PROTACs which do facilitate cooperative ternary complexes have a broaderwindow of degradation activity as the complex equilibria is now shifted towards the ternary complex as compared to the non-productive binary interactions of compounds with either the target or E3 ligase (FIG. 5b). The downstream ubiquitination and degradation outcomes will be ternary complex dependent and therefore could potentially be optimized through rational chemical design¹⁵.

The studies with the trivalent PROTAC disclosed herein provide evidence to support optimization of structural, energetic and kinetic ternary complex favourability parameters as a result of increased avidity in the process (FIG. 5b). This is most notably observed with the prolonged cellular residence time of compound SIM1 binding to BRD4, only when VHL can engage to form the ternary complex. An improvement in all the parameters with the trivalent PROTAC resulted in a vast expansion of the degradation window, from rapid rates of BET family loss at very low concentration to maximal degradation with no hook effect observed at higher concentration 10,000-fold above the Dmax₅₀. Some of these characteristics have been observed previously with bivalent BET family PROTACs, such as cooperative complexes with MZ1⁹ and potent degradation with dBET-6³⁸ and ARV-771³⁷, but the combination of these to achieve maximal avidity in the ternary complex to drive profound degradation had not yet been shown.

The transition from heterobifunctional degraders to trivalent degraders is not an obvious approach for improvement of degradation outcomes, particularly given the chemical synthesis challenges and presupposition that increasing molecular weight of degraders would be accompanied by either decreased cellular permeability or a completely impermeable compound. Surprisingly, the compounds of formula I disclosed and characterized herein, demonstrated that this is not the case. While indeed the trivalent compounds SIM1 and cis-SIM1 have reduced permeability compared to their parent bivalent inhibitor, they are still readily cell-permeable.

In addition, the transition from bivalent BET PROTACs to trivalent ones resulted in several highly potent and efficacious compounds, improving degradation of all BET family members as well as performance in relevant cellulardisease assays used for assessment of BET compound potential fortherapeutic use.

While the chemical design and synthesis of a trivalent degrader is more involved than for bivalent PROTACs, the increased effort showed significant benefits and outlined is a new linker design strategy for generation of a branched trifunctional scaffold to which both target and E3 binders could be conjugated. In overcoming these perceived challenges, the trivalent PROTAC revealed that increasing target binding valency afforded an improved degrader.

### Biological Methods

Cell lines and culture. HEK293, 22Rv1 and MV4; 11 cells (ATCC) were grown in DMEM and RPMI (Invitrogen) respectively and supplemented with 10% v/v foetal bovine serum (FBS) (South American origin, Life Science Production) at 37° C. and 5% CO₂ in a humidified atmosphere. CRISPR HiBiT-BRD2, HiBiT-BRD3, and HiBiT-BRD4 HEK293 cells stably expressing LgBiTwere grown in DMEM with 10% v/v FBS and CRISPR cMyc-HiBiT MV4; 11 cells were grown in RPMI with 10% v/v FBS. All cells were split 1-2 times per week when 90% confluent and were not used beyond passage 30. Cells were routinely checked for mycoplasma contamination using Mycoalert detection kit (Lonza).

Degradation assays. MV4; 11 cells were seeded at 1×10⁶ cells/mL of 10 cm dishes 12-24 h before treatment. 22Rv1 and HEK293 cells were seeded at 2.5-6×10⁵ cells/well of 6 well plates 12-24 h before treatment. Cells were treated with test compounds with and without inhibitors as indicated or an equivalent volume of DMSO and lysed at the stated time point. For lysis, cells were washed twice in ice cold PBS (Invitrogen) then lysed in 250 μL/plate for MV4; 11 cells or 80 μL/well for 22Rv1 and HEK293 cells of ice cold lysis buffer containing 50 mM Tris hydrochloride pH 7.4, 150 mM sodium chloride, 1 mM EDTA pH 7.4, 1% v/v Triton X-100, 1×Halt™ Protease Inhibitor Cocktail (ThermoFisher). Lysates were sonicated, cleared by centrifugation at 4° C., at 15800×g for 10 mins and the supernatants stored at −80° C. Protein concentration was determined by BCA assay (Pierce) and the absorbance at 562 nm measured by spectrophotometry (NanoDrop ND1000). Samples were run on SDS-PAGE using NuPAGE Novex 4-12% Bis-Tris gels (Invitrogen) with 40 μg total protein/well, transferred to 0.2 μm pore nitrocellulose membrane (Amersham) by wet transfer and blocked with 3% w/v BSA (Sigma) in 0.1% TBST. Blots were incubated in anti-BRD2 (1:2000, abcam #ab139690), anti-BRD3 (1:500, abcam #ab50818), anti-BRD4 (1:1000, abcam #ab128874), anti-c-myc (1:1000, abcam #32072), anti-PARP (1:1000, CST #9542S), anti-cleaved PARP (1:1000, BD Pharmingen #51-9000017), anti-caspase-3 (1:1000, CST #9662S), anti-tubulin (1:3,000, Bio-Rad #12004165) or anti-β-actin (1:2500, CTS #4970S) antibody overnight at 4° C. with rotation. Blots were then incubated in goat anti-mouse or donkey anti-rabbit IRDye 800CW secondary antibodies (1:10,000, LICOR #925-32210 and #926-32213) for 1 h at room temperature with rotation. Bands were detected using a ChemiDoc MP imaging system (BioRad) and quantified (Image Studio Lite, version 5.2) with normalisation to β-actin and the DMSO control per time point. Data are the average of two biological repeats unless indicated otherwise. Degradation data were plotted and fitted by nonlinear regression using a single-phase exponential decay model in Prism (Graphpad, version 7.03).

Cell Viability Assay. MV4; 11 cells were incubated with compounds at the desired concentration for 48 h on a clear-bottom 384-well plate. MV4; 11 cells were kept in RPMI medium supplemented with 10% FBS, L-glutamine. Initial cell density was 3×10⁵ per mL. The cells were treated with various concentrations of compound or 0.05% DMSO in triplicates for each concentration point. After treatment, cell viability was measured with Promega CellTiter-Glo luminescent cell viability assay kit according to the manufacturer instructions. Signal was recorded on a BMG Labtech Pherastar luminescence plate reader with recommended settings. Data were analysed with Graphpad Prism software to obtain EC₅₀ values of each test compound.

Kinetic Degradation and Quantitation. HEK293 cells (ATCC) stably expressing LgBiT (Promega) were edited using CRISPR/Cas9 to endogenously HiBiT tag the N-terminal genomic loci of BRD2, BRD3, or BRD4³¹. For kinetic degradation assays, cells were plated in white 96-well tissue culture plates at a density of 2×10⁴ cells per well in 100 μL of growth medium and incubated overnight at 37° C., 5% CO₂. The following day, medium was replaced with CO₂-independent medium (Gibco) containing a 1:100 dilution of Endurazine (Promega) and were incubated at 37° C. in 5% CO₂ for 2.5 h before addition of a 3-fold serial dilution of 1 μM (SIM1, SIM2 and SIM3 with BRD4) or 10 nM (SIM1 with BRD2, 3, and 4) final concentration of compound. Plates were placed with lids into the GloMax Discover Microplate Reader (Promega) set to 37° C. and continuous luminescent measurements with readings every 5-15 min were carried out over a 24 h period post-compound treatment.

Quantitation of Degradation Kinetics. Degradation rate and degradation plateau were calculated³¹ from above determined kinetic degradation profiles. Briefly, the initial degradation portion of each kinetic concentration curve was fitted to the equation:

y=(y₀−plateau)e⁻+plateau

where λ=degradation rate in units of h⁻¹. The degraded fraction, Dₘₐₓ, was calculated as 1−plateau. For each curve, the data points before onset of degradation were excluded from the fits. The Dₘₐₓ was then plotted against concentration to determine D_{max 50} values.

Mass spectrometry proteomics. Sample preparation. MV4; 11 cells in RPMI (Invitrogen) were seeded at 5×10⁸ cells on a 100 mm plate 24 h before treatment. Cells were treated in triplicate by addition of test compound. After 4 h, the cells were centrifuged at 250 g for 5 min and washed twice with 12 mL of cold PBS. Cells were lyzed in 500 μL of 100 mM TRIS pH 8.0, 4% (w/v) SDS supplemented with protease inhibitor cocktail (Roche). The lysate was pulse sonicated briefly and then centrifuged at 15,000 g for 10 min at 4° C. Samples were quantified using a micro BCA protein assay kit (Thermo Fisher Scientific) and 200 μg of each sample was processed and digested using the filter aided sample preparation method followed by alkylation with iodoacetamide and digestion with trypsin as previously described²¹. The samples were then desalted using a 7 mm, 3 mL C18 SPE cartridge column (Empore, 3 M) and labelled with TMT 10plex Isobaric Label Reagent Set (Thermo Fisher Scientific) as per the manufacturer's instructions. After labelling, the peptides from the nine samples were pooled together in equal proportion. The pooled sample was fractionated using high pH reverse-phase chromatography on an XBridge peptide BEH column (130 Å, 3.5 μm, 2.1×150 mm, Waters) on an Ultimate 3000 HPLC system (Thermo Scientific/Dionex). Buffers A (10 mM ammonium formate in water, pH 9) and B (10 mM ammonium formate in 90% acetonitrile, pH 9) were used over a linear gradient of 5 to 60% buffer B over 60 min at a flow rate of 200 μL min⁻¹. Then, 80 fractions were collected before concatenation into 20 fractions on the basis of the ultraviolet signal of each fraction. All the fractions were dried in a Genevac EZ-2 concentrator and resuspended in 1% formic acid for mass spectrometry analysis.

LC-MS/MS analysis. The fractions were analysed sequentially on a Q Exactive HF Hybrid Quadrupole-Orbitrap Mass Spectrometer (Thermo Scientific) coupled to an UltiMate 3000 RSLCnano ultra HPLC system (Thermo Scientific) and EasySpray column (75 μm×50 cm, PepMap RSLC C18 column, 2 μm, 100 Å, Thermo Scientific). Buffers A (0.1% formic acid in water) and B (0.08% formic acid in 80% acetonitrile) were used over a linear gradient from 5 to 35% buffer B over 125 min at 300 nL min⁻¹. The column temperature was 50° C. The mass spectrometer was operated in data dependent mode with a single mass spectrometry survey scan from 335-1,600 m/z followed by 15 sequential m/z dependent MS2 scans. The 15 most intense precursor ions were sequentially fragmented by higher energy collision dissociation. The MS1 isolation window was set to 0.7 m/z and the resolution set at 120,000. MS2 resolution was set at 60,000. The automatic gain control (AGC) targets for MS1 and MS2 were set at 3×10⁶ ions and 1×10⁵ ions, respectively. The normalized collision energywas set at 32%. The maximum ion injection times for MS1 and MS2 were set at 50 and 200 ms, respectively.

Peptide and protein identification. The raw mass spectrometry data files for all 20 fractions were merged and searched against the Uniprot-sprot-Human-Canonical database by MaxQuant software v.1.6.0.16 for protein identification and TMT reporter ion quantitation. The MaxQuant parameters were set as follows: enzyme used trypsin/P; maximum number of missed cleavages equal to two; precursor mass tolerance equal to 10 ppm; fragment mass tolerance equal to 20 ppm; variable modifications: oxidation (M), acetyl (N-term), deamidation (NQ), Gln→pyro-Glu (Q N-term); fixed modifications: carbamidomethyl (C). The data was filtered by applying a 1% false discovery rate followed by exclusion of proteins with fewer than two unique peptides. Quantified proteins were filtered if the absolute fold-change difference between the three DMSO replicates was ≥1.5.

Monitoring cMyc Loss and Cell Viability in MV4; 11 Cells. CRISPR cMyc-HiBiT MV4; 11 cells (Promega) were plated at a density of 5×10⁴ cells per well in solid, white 96-well tissue culture plates (Corning Costar #3917). Following an overnight incubation, they were treated with 1-100 nM concentration of the indicated compounds and at the plotted time points, cMyc levels were determined using luminescent measurement with NanoGlo HiBiT lytic reagent (Promega). Replicate plate of all compound treatments were prepared and at identical timepoints as the protein level measurements, cell viability was measured using Cell-Titer Glo (Promega). Plates were shaken on an orbital shaker for 10-20 min before reading luminescence on a GloMax Discover Microplate Reader (Promega).

Caspase-Glo® 3/7 assays. 22Rv1 cells were seeded at 10,000 cells/well of white 96 well plates (Corning #3917) 12-24 h before treatment with test compounds with and without inhibitors or an equivalent volume of DMSO for 24 h. 100 μL/well of Caspase-Glo@ 3/7 Reagent (Promega) was added and the plate shaken at 500 rpm for 30 seconds. The plate was incubated for 2 h and luminescence measured using a PHERAstar FS plate reader (BMG Labtech).

Clonogenic assay. 22Rv1 cells were treated with 10 nM SIM1, cis-SIM1, MT1, MZ1 and ARV711 for 24 h. The next day, cells were trypsinised and counted. 500 cells were re-plated and allowed to grow at 37° C. and 5% CO₂ for 20 days. After 20 days incubation, the cells were fixed with ice-cold 100% (v/v) methanol for 30 min at 4° C. Afterwards, methanol was removed and the cells were stained with 500 μl 0.1% crystal-violet dye (in MeOH) for 30 min at room temperature. Following incubation, the cells were washed with dH₂O and left to dry overnight. Plates were scanned on a Epson Perfection V800 Photo scanner. And image analysis was done in ImageJ software. Plating efficiency (PE) was calculated by counting colonies for each treatment condition and dividing the average by number of cells plated. Survival fraction was determined by diving PE of treated cells by PE of untreated cells, multiplied by 100. Bar graphs were generated using GraphPad prism software. Error bars indicate the mean values ±S.D. Two independent experiments were performed.

Constructs, protein expression and purification. Wild-type and mutant versions of human proteins BRD2 (P25440), BRD3 (Q15059) and BRD4 (060885) VHL (UniProt accession number: P40337), ElonginC (Q15369), ElonginB (Q15370), were used for all protein expression.

pET-His-SUMO TEV BRD4 tandem was produced by cloning truncated BRD4 containing the two bromodomains (residue 1-463) into parent pET His6 Sumo TEV LIC cloning vector (1S) using ligase independent cloning. pET His6 Sumo TEV LIC cloning vector (1S) was a gift from Scott Gradia (Addgene plasmid #29659). Quikchange mutagenesis was performed on pET-His-SUMO BRD4 tandem with mutagenic primer following standard procedures, to generate mutant with the conserved Asparagine sitting in the Acetyl-lysine binding pocket substituted with Phenylalanine, i.e. BRD4 N140F and BRD4 N433F.

For expression of BRD4 tandem construct, N-terminally His6-tagged BRD4 (1-463) or similar mutants were expressed in Escherichia coli BL21(DE3) at 18° C. for 16 h using 0.4 mM isopropyl β-D-1-thiogalactopyranoside (IPTG). E. coli cells were lysed using a pressure cell homogenizer (Stansted Fluid Power) and lysate clarified by centrifugation. His6-tagged VCB was purified on a HisTrap HP affinity column (GE Healthcare) by elution with an imidazole gradient. The His6 tag was removed using TEV protease and the untagged complex dialyzed into low-concentration imidazole buffer. BRD4 was then flowed through the HisTrap HP column a second time, allowing impurities to bind, as the complex eluted without binding. BRD4 was then additionally purified by anion exchange and size-exclusion chromatography using Mono S and Superdex-200 columns (GE Healthcare), respectively. The final purified complex was stored in 20 mM HEPES, pH 7.5, 100 mM sodium chloride and 1 mM TCEP.

The VCB complex was expressed and purified as described previously. Briefly, N-terminally His6-tagged VHL (54-213), ElonginC (17-112) and ElonginB (1-104) were co-expressed and the complex was isolated by Ni-affinity chromatography, the His6 tag was removed using TEV protease, and the complex further purified by anion exchange and size-exclusion chromatography.

The BET protein BDs were expressed and purified as described previously²¹. Briefly, N-terminally His6-tagged BRD2-BD1 (71-194), BRD2-BD2 (344-455), BRD3-BD1 (24-146), BRD3-BD2 (306-416), BRD4-BD1 (44-178) and BRD4-BD2 (333-460) were expressed and isolated by Ni-affinity chromatography and size-exclusion chromatography.

Size exclusion chromatography (SEC). SEC experiments were carried out in a ÄKTA pure system (GE Healthcare) at room temperature. The oligomeric state of the BRD4 BD1 BD2 tandem protein in solution was analysed by gel filtration in a buffer containing 20 mM HEPES (pH 7.5), 100 mM NaCl and 1 mM TCEP using a Superdex 200 Increase 10/300 GL column (GE Healthcare) calibrated with globular proteins of known molecular weight (GE Healthcare, 28-4038-41/42). BRD4 tandem (25 μM) was incubated with SIM1 (25 μM), MZ1 (25 μM), MT1 (25 μM) or DMSO (0.5%) for 30 min at room temperature prior to injection. Sample volume for each injection was 200 μl, and the flow rate was 0.8 ml/min. Peak elution was monitored using ultraviolet absorbance at 280 nm.

ITC. Titrations were performed on an ITC200 micro-calorimeter (Malvern). The titrations consisted of 19 injections of 2 μl tandem BRD4 BD1-BD2 construct (WT or N140F or N433F) solution in 20 mM Bis-Tris propane, 100 mM NaCl, 1 mM TCEP, 1.6% DMSO, pH 7.5, at a rate of 0.5 μl/s at 120 s time intervals. An initial injection of protein (0.4 μl) was made and discarded during data analysis. All experiments were performed at 25° C., whilst stirring at 750 r.p.m. SIM1 from 10 mM DMSO stock solution and VCB were diluted in buffer containing 20 mM Bis-Tris propane, 100 mM NaCl, 1 mM TCEP, pH 7.5. The final DMSO concentration was 1.6% v/v. BRD4 protein (200 μM, in the syringe) was titrated into the SIM1-VCB complex (SIM1 16 μM, VCB 32 μM, in the cell). Data were fitted to a single-binding site model for each BRD4 mutant or a two-binding site model for BRD4 WT to obtain the stoichiometry (n), the dissociation constant (Kd) and the enthalpy of binding (ΔH) using Microcal LLC ITC200 Origin software provided by the manufacturer.

AlphaLISA assays. Ligands were titrated against 4 nM His-tagged BRD4 BD2 and 10 nM biotinylated JQ1. All reagentswere diluted in 50 mM HEPES, 100 mM NaCl, 0.1% BSA, 0.02% CHAPS, pH7.5 (final concentration). On VCB premixed condition, the buffer also included 12.5 mM VCB. Ligands were tested over an 11-point 3-fold serial dilution, starting at 100 μM without VCB or starting at 10 mM with 20 mM VCB, and giving a final DMSO concentration of 1%. Binding was detected using anti-His6 antibody-conjugated AlphaLISA acceptor beads and streptavidin-coated donor beads (PerkinElmer), with a final concentration of 10 μg/ml for each bead). Titrations were prepared in white 384-well Alphaplates (PerkinElmer), and read on a Pherastar FS plate reader (BMG) equipped with an AlphaLISA excitation/emission module. Data was analysed and dose-response curves generated using GraphPad Prism 7. Each assay well had a final volume of 25 μl. First 10 μl of 2.5×ligand or 2.5× ligand with VCB was mixed with 5 μl of a 5× mix of bromodomain and biotinylated JQ1 and incubated for 1 h at room temperature. The assay plate was then moved to a dark room and 5 μl of 5× acceptor beads were added and incubated for 1 h. Then (still in darkness) 5 μl of 5× donor beads were added, the plate was incubated for 1 h before being read.

Fluorescence polarization assay. FP competitive binding assays were run as described previously³² with a final volume of 15 μL, with each well solution containing 15 nM VCB protein, 10 nM FAM-labelled HIF-1α peptide (FAM-DEALAHypYIPMDDDFQLRSF, “JC9”) and decreasing concentrations of PROTAC (14-point 2-fold serial dilution starting from 50 μM) or PROTAC:BRD4 tandem protein (14-point 2-fold serial dilutions starting from 10 μM PROTAC:40 μM individual bromodomain or 10 μM PROTAC:20 μM tandem bromodomain). Assays were prepared in triplicate on 384-well plates (Corning 3575) and all measurements taken using a PHERAstar FS (BMG LABTECH) with fluorescence excitation and emission wavelengths (λ) of 485 and 520 nm, respectively. Components were dissolved from stock solutions using 100 mM Bis-Tris propane, 100 mM NaCl, 1 mM TCEP, pH 7.5, and DMSO was added as appropriate to ensure a final concentration of 1%. Control wells containing VCB and JC9 with no compound (zero displacement), or JC9 in the absence of protein (maximum displacement) were also included to allow for normalization. Normalized (%) displacement values were plotted against log[PROTAC] and curves were fitted by nonlinear regression using Prism (v. 8.0.1, GraphPad) to determine the IC₅₀ values for each titration. Kᵢ values were back-calculated from the K_{d} for JC9 (˜2 nM, determined from direct binding) and fitted IC₅₀ values, as described previously. Cooperativity (α) values were calculated from the ratio of binary Kᵢ and ternary Kᵢ values determined for JC9 displacement by SIM1 alone or SIM1+BRD4, respectively.

SPR binding studies. SPR experiments were performed on Biacore T200 instruments (GE Healthcare) as described previously³². Immobilization of Biotinylated VCB was carried out at 25° C. on a pre-coupled Series S SA chip in running buffer containing 20 mM TRIS, 150 mM potassium chloride, 2 mM magnesium chloride, 2 mM TCEP, 0.005% TWEEN 20, 1% DMSO; pH 8.3. Multiple surface densities of biotinylated VHL were used (40, 80 and 120 RU). Biotinylated VCB was prepared as previously described³². All interaction experiments were performed at 9° C. in running buffer containing 20 mM TRIS, 150 mM potassium chloride, 2 mM magnesium chloride, 2 mM TCEP, 0.005% TWEEN 20, 1% DMSO; pH 8.3. SIM1 (10 mM in 100% DMSO) were initially prepared at 1 μM in running buffer with a concentration of 2% DMSO. This solution was mixed 1:1 with a solution of 50 μM of the BRD4 tandem protein in running buffer without DMSO, to prepare a final solution of 500 nM SIM1 and 25 μM BRD4 tandem protein in running buffer containing 1% DMSO. This complex was then serially diluted in running buffer containing 2 μM bromodomain and 1% DMSO (5-point five-fold serial dilution). Solutions were injected sequentially in single-cycle kinetic format without regeneration (three replicate series per experimental repeat, contact time 100 sec, flow rate 100 μL/min, dissociation time 800 sec) using a stabilization period of 30 sec and syringe wash (50% DMSO) between injections. High flow rates and multiple surface densities were used to minimise mass transfer effects. Two series of blank injections were performed for all single cycle experiments. Sensorgrams from reference surfaces and blank injections were subtracted from the raw data before data analysis using Biacore insight evaluation software. To calculate the association rate (kₒₙ), dissociation rate (k_{off}), and dissociation constant (K_{D}), experiments were fitted using a 1:1 Langmuir interaction model, with a term for mass-transport included.

NanoBRET Ubiquitination, Ternary Complex, and Biosensor Experiments. For endogenous live cell BET:Ubiquitin and BET:VHL assays, CRISPR HiBiT-BRD2, HiBiT-BRD3, and HiBiT-BRD4 HEK293 cells stably expressing LgBiT were transfected with 2 μg of HaloTag-UBB or HaloTag-VHL vectors in 6-well plates using FuGENE HD (Promega). For full transient NanoBRET experiments with NanoLuc-BRD4 WT, N433F, or N140F mutants, HEK293 cells (8×10⁵) were co-transfected with 0.02 μg NanoLuc-BRD4 and 2 μg of HaloTag-VHL vectors. For transient NanoBRET experiments with the BRD4 NL-BD1-BD2-HT biosensor containing either the WT tandem BD1-BD2 domain (AA 44-460) or containing the N433F mutation, HEK293 cells (8×10⁵) were transfected with 0.02 μg biosensor plasmid and 2 μg carrier DNA. The following day, transfected cells (2×10⁴) were replated into white 96-well tissue culture plates in the presence or absence of HaloTag NanoBRET 618 Ligand (Promega) and incubated overnight at 37° C., 5% CO₂. For kinetic experiments, medium was replaced with Opti-MEM (Gibco) containing a 1:100 dilution of Vivazine (Promega), and plates were incubated at 37° C., 5% CO₂, for 1 h before addition of DMSO or 10 nM-1 μM final concentration of the indicated compounds. Continual BRET measurements were then made every 3 min up to 5 h on a CLARIOstar equipped with an atmospheric control unit (BMG Labtech) set to 37° C. and 5% CO₂. For the biosensor experiments, the cells were treated with a 3-fold serial titration of 10 μM of the indicated compounds. NanoBRET NanoGlo (Promega) substrate was added and BRET was measured two hours post-compound treatment using a GloMax Discover Microplate Reader (Promega). Dual filtered luminescence was collected with a 460/80 nm bandpass filter and a 610 nm long pass filter (acceptor, HaloTag NanoBRET ligand) using an integration time of 0.5 s. For all NanoBRET experiments, background subtracted NanoBRET ratios expressed in milliBRET units were calculated from the equation:

$$mBRET ratio = ( \frac{acceptor channel}{donor channel} - \frac{acceptor channel ( no ligand )}{donor channel ( no ligand )} ) 1000.$$

Fold increase in BRET was calculated by normalizing mBRET ratios to the average mBRET ratios for DMSO controls.

NanoBRET Target Engagement and Residence Time. For target engagement experiments in live and permeabilized cells, CRISPR HiBiT-BRD4 HEK293 cells stably expressing LgBiT were plated into white 96-well tissue culture plates at a density of 2×10⁴ cells/well. Cells were equilibrated for 1 h with energy transfer probes and the indicated test compound prior to NanoBRET measurements. NanoBRET tracers were prepared at a working concentration of 20× in tracer dilution buffer (12.5 mM HEPES, 31.25% PEG-400, pH 7.5). NanoBRET BRD Tracer-02 was added to cells at a final concentration of 0.5 μM. To measure NanoBRET in live cells, NanoBRET NanoGlo Substrate and Extracellular NanoLuc Inhibitor (Promega) were added according to the manufacturer's recommended protocol, and filtered luminescence was measured on a GloMax Discover luminometer equipped with 450 nm BP filter (donor) and 600 nm LP filter (acceptor) using 0.3 s integration time. To measure NanoBRET in permeabilized cells, digitonin was added to the cells to a final concentration of 50 μg/mL and Extracellular NIuc inhibitor was omitted during the detection step. For residence time experiments CRISPR HiBiT-BRD4 HEK293 cells stably expressing LgBiT were trypsinized, washed and resuspended to a density of 2×10⁵ cells/ml in Opti-MEM and incubated with either 1 μM JQ1, 1 μM SIM1, 100 nM cis-SIM1, 100 nM MT1, or 10 μM MZ1 representing the representative IC80 values for tracer displacement in live cell format. Cells were incubated in 15 mL conical tubes with caps loosened in a tissue culture incubator for 1 h. Following incubation, cells were spun at 300×g for 5 min, washed once with Opti-MEM, spun a second time at 300×g for 5 min, then resuspended with fresh Opti-MEM before plating at 2×10⁴ cells/well. NanoBRET BRD Tracer-02 was added at a final concentration of 0.5 μM cells and kinetic NanoBRET measurements were collected on a GloMax Discover. NanoBRET ratios were expressed in milliBRET units and calculated according to the equation in the NanoBRET Ubiquitination, Ternary Complex and Biosensor Experiments section.

### Chemistry—Materials and Methods

All chemicals unless otherwise stated, were commercially available, at least 90% pure and used without further purification. Commercially available dry solvents were used. Normal phase TLC was carried out on pre-coated silica plates (Kieselgel 60 F254, BDH) with visualization via UV light (UV 254 and/or 365 nm) and/or basic potassium permanganate solution. Flash column chromatography was performed using a Teledyne Isco Combiflash Rf with prepacked Redisep RF Normal phase disposable Columns. NMR Spectra were recorded on a Bruker Ascend 400 MHz or 500 MHz as specified. Chemical shifts are quoted in ppm and referenced to the residual solvent signals: ¹H NMR δ (ppm)=7.26 (CDCl₃), ¹³C NMR δ (ppm)=77.16 (CDCl₃); ¹H NMR δ (ppm)=5.32 (CD₂Cl₂), ¹³C NMR δ (ppm)=53.84 (CD₂Cl₂), ¹H NMR δ (ppm)=2.50 (DMSO-d6); ¹H NMR δ (ppm)=3.31 (CD₃OD), ¹³C NMR δ (ppm)=49.00 (CD₃OD). Signal splitting patterns are described as singlet (s), doublet (d), triplet (t), quartet (q), multiplet (m), broad (br) or a combination thereof. Coupling constants (J) are measured in Hertz (Hz). High Resolution Mass Spectra (HRMS) were recorded on a Bruker microTOF. Other resolution MS and analytical HPLC traces were recorded on an Agilent Technologies 1200 series HPLC connected to an Agilent Technologies 6130 quadrupole LC/MS, connected to an Agilent diode array detector. The column used was a Waters XBridge column (50 mm×2.1 mm, 3.5 μm particle size) and the compounds were eluted with a gradient 5-95% acetonitrile/water+0.1 formic acid (“acidic method”). HPLC purification was performed on a Gilson Preparative HPLC System with a Waters XBridge C18 column (100 mm×19 mm; 5 μm particle size) and a gradient of 5% to 95% acetonitrile in water over 10 min, flow 25 mL/min, with 0.1% ammonia in the aqueous phase.

Abbreviations used: DMSO for dimethylsulfoxide, PMB for p-methoxybenzyl, Ms for mesyl, tBu for tert-butyl, pTsOH for p-toluenesulfonic acid, TBAB for tetrabutylammonium bromide, TFA for trifluoroacetic acid, MeOH for methanol, DCM for dichloromethane, DDQ for 2,3-Dichloro-5,6-dicyano-1,4-benzoquinone, THE for tetrahydrofuran, HATU for 1-[bis(dimethylamino)methylene]-1H-1,2,3-triazolo[4,5-b]pyridinium 3-oxid hexafluorophosphate, HOAt for 1-hydroxy-7-azabenzotriazole, DIPEA for N,N-diisopropylethylamine, DMF for N,N-dimethylformamide, MTBE for methyl tert-butyl ether, COMU for (1-cyano-2-ethoxy-2-oxoethylidenaminooxy)dimethylamino-morpholino-carbenium hexafluorophosphate.

### Synthesis of Compounds SIM1, SIM2, SIM3

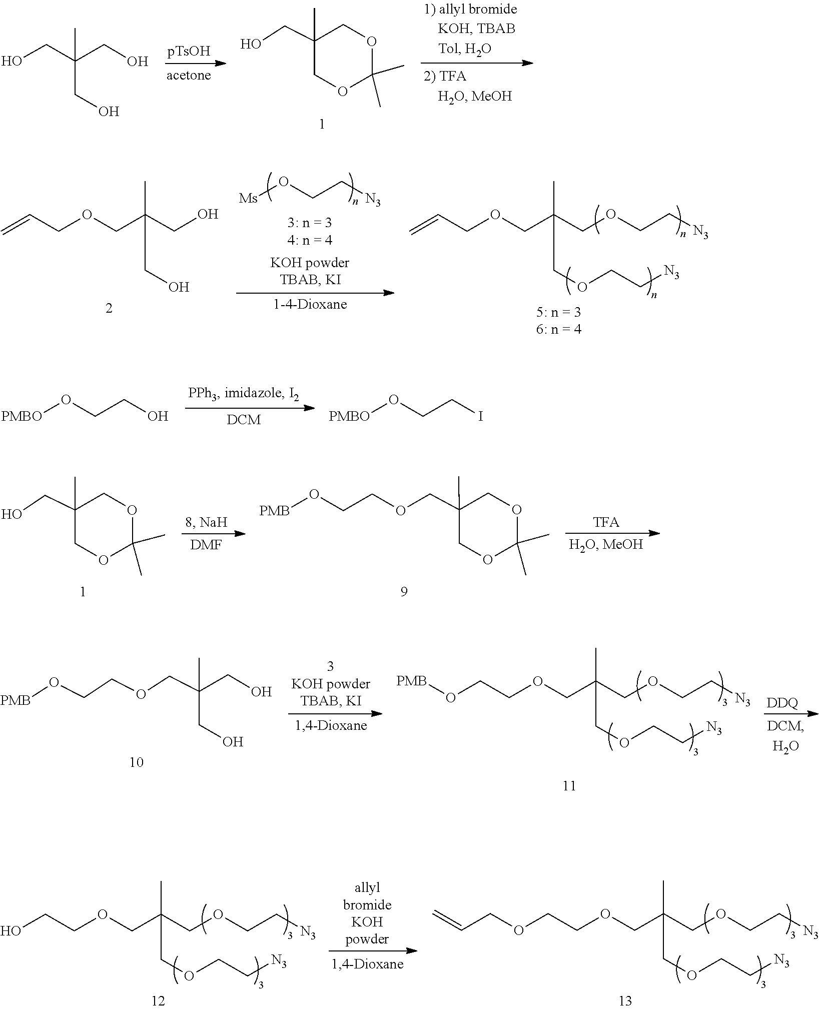

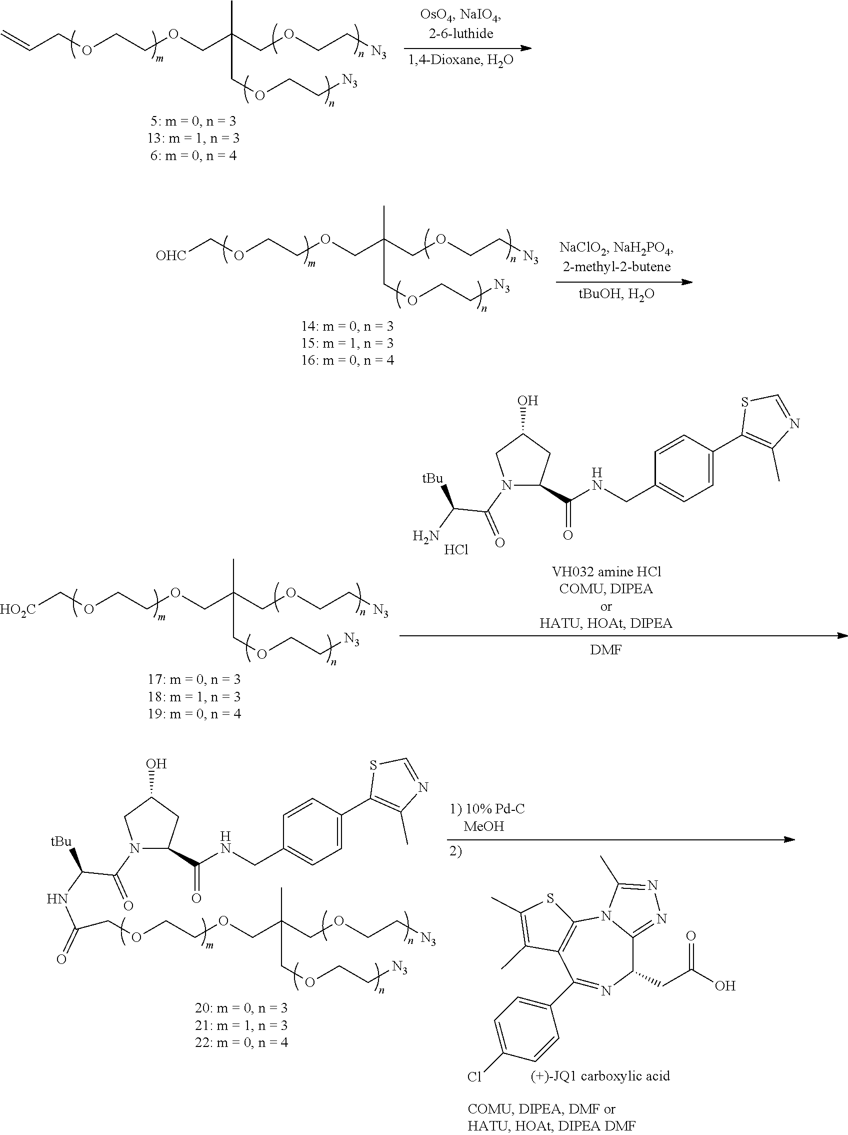

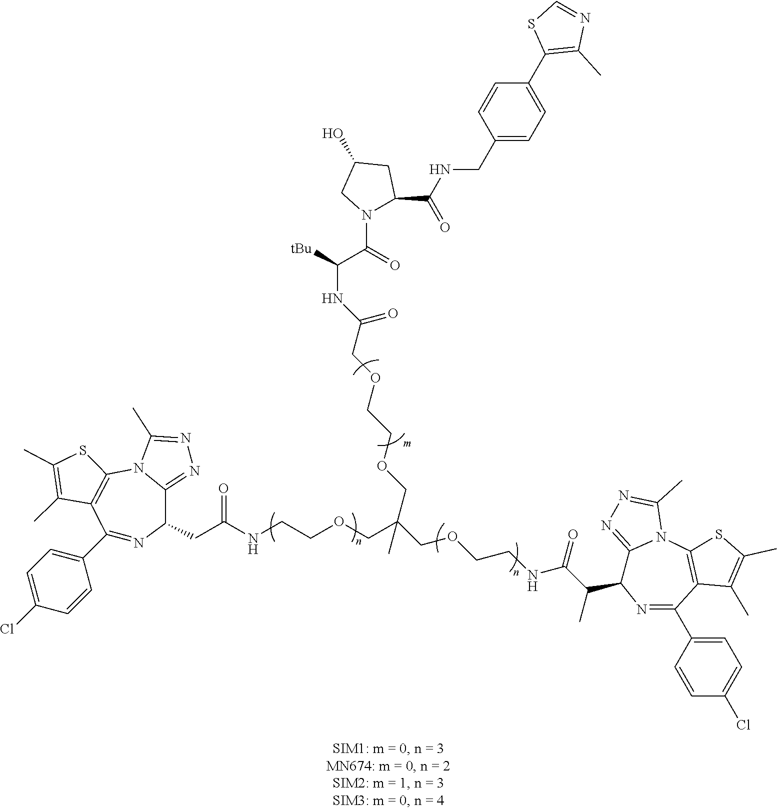

### (2,2,5-trimethyl-1,3-dioxan-5-yl) methanol (1)

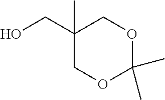

2-(Hydroxymethyl)-2-methylpropane-1,3-diol (50.0 g, 0.42 mol) and p-toluenesulfonic acid (50 mg) were dissolved in dry acetone (500 ml). The mixture was stirred for 2 days at room temperature. The solution was neutralized by adding solid potassium carbonate, filtrated, and evaporated under vacuum to give the desired product (64 g, 96%, thick colourless oil) which was used without any further purification. Analytical data matched those reported in literature (Ouchi M. et al. J. Org. Chem. 1987, 52, 2420).

### 2-((allyloxy)methyl)-2-methylpropane-1,3-diol (2)

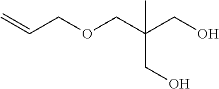

Potassium hydroxide (1.05 g, 16.75 mmol) in H₂O (1.05 ml), allyl bromide (1.54 ml, 18.75 mmol) and TBAB (202 mg, 0.625 mmol) were added to a solution of (2,2,5-trimethyl-1,3-dioxan-5-yl) methanol 1 (1.0 g, 6.25 mmol) in toluene (6.25 ml). The resulting mixture was stirred at r.t. for 24 h. The reaction mixture was diluted with dichloromethane. The organic phase was separated and evaporated to dryness. The crude material was purified by column chromatography. The resulting allyl ether was dissolved in methanol (16 ml) and H₂O (3.2 ml). After added trifluoroacetic acid (287 μL), the mixture was stirred at r.t. for 4 h. The reaction mixture was evaporated to dryness. The crude material was purified by column chromatography to afford title compound. Yield: 860 mg (86%).

¹H NMR (400 MHz, CDCl₃) δ (ppm)=5.98-5.83 (1H, m), 5.28 (1H, dd, J=1.4, 17.3 Hz), 5.21 (1H, dd, J=1.2, 10.5 Hz), 4.03-3.98 (2H, m), 3.73 (2H, dd, J=4.8, 11.0 Hz), 3.62 (2H, dd, J=5.5, 10.9 Hz), 3.47 (2H, d, J=4.0 Hz), 2.70 (2H, s), 0.86 (3H, s). ¹³C NMR (101 MHz, CDCl₃) δ (ppm)=134.3, 117.2, 75.8, 72.5, 68.0, 40.8, 17.2.

### 11-((allyloxy)methyl)-1,21-diazido-11-methyl-3,6,9,13,16,19-hexaoxahenicosane (5)

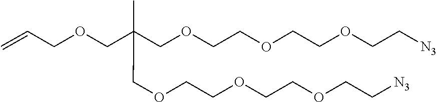 [SMILES: C=CCOCC(C)(COCCOCCOCCN=[N+]=[N-])COCCOCCOCCN=[N+]=[N-]]

2-((allyloxy)methyl)-2-methylpropane-1,3-diol 2 (136 mg, 0.85 mmol), 2-(2-(2-azidoethoxy)ethoxy)ethyl methanesulfonate 3 (646 mg, 2.55 mmol) were dissolved in 1,4-dioxane (0.85 mL). TBAB (55 mg, 0.18 mmol), potassium iodide (7.1 mg, 0.04 mmol) and potassium hydroxide powder (143 mg, 2.55 mmol) were added and the reaction was stirred at 100° C. for 2 h. The reaction mixture was diluted with dichloromethane and filtrated. The organic phase was evaporated to dryness. The crude material was purified by HPLC under acidic condition (5-95% CH₃CN in 0.1% aq. HCO₂H) to afford title compound. Yield: 50 mg (12%).

¹H NMR (500 MHz, CDCl₃) δ (ppm)=5.96-5.84 (1H, m), 5.26 (1H, dd, J=1.7, 17.2 Hz), 5.15 (1H, dd, J=1.5, 10.4 Hz), 4.01-3.93 (2H, m), 3.76-3.61 (16H, m), 3.62-3.55 (4H, m), 3.44-3.37 (4H, m), 3.37-3.33 (4H, m), 3.33-3.29 (2H, m), 0.96 (3H, s).

¹³C NMR (126 MHz, CDCl₃) δ (ppm)=135.3, 116.0, 74.0, 73.0, 72.3, 71.1, 70.8, 70.7, 70.5, 70.0, 50.7, 41.0, 17.4.

### 14-((allyloxy)methyl)-1,27-diazido-14-methyl-3,6,9,12,16,19,22,25-octaoxaheptacosane (6)

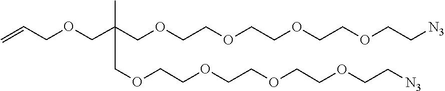 [SMILES: C=CCOCC(C)(COCCOCCOCCOCCN=[N+]=[N-])COCCOCCOCCOCCN=[N+]=[N-]]

2-((allyloxy)methyl)-2-methylpropane-1,3-diol 2 (50 mg, 0.31 mmol), 2-(2-(2-(2-azidoethoxy)ethoxy)ethoxy)ethyl methanesulfonate 4 (278 mg, 0.94 mmol) were dissolved in 1,4-dioxane (0.31 mL). TBAB (55 mg, 0.18 mmol), potassium iodide (2.6 mg, 0.016 mmol) and potassium hydroxide powder (52.5 mg, 0.94 mmol) were added and the reaction was stirred at 100° C. for 2 h. The reaction mixture was diluted with dichloromethane and filtrated. The organic phase was evaporated to dryness. The crude material was purified by HPLC under acidic condition (5-95% CH₃CN in 0.1% aq. HCO₂H) to afford title compound. Yield: 15 mg (9%).

¹H NMR (500 MHz, CDCl₃) δ (ppm)=5.93-5.83 (1H, m), 5.25 (1H, dd, J=1.3, 17.2 Hz), 5.16-5.11 (1H, m), 3.96-3.92 (2H, m), 3.71-3.54 (28H, m), 3.42-3.27 (10H, m), 0.94 (3H, s).

¹³C NMR (126 MHz, CDCl₃) δ (ppm)=135.5, 116.2, 74.2, 73.2, 72.4, 71.2, 70.9, 70.8, 70.6, 70.2, 50.9, 41.2, 17.6.

### 1-((2-iodoethoxy)methyl)-4-methoxybenzene (8)

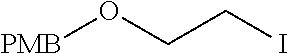

Iodine (180 mg, 0.71 mmol) was added at 0° C. to triphenylphosphine (187 mg, 0.71 mmol) and imidazole (48.6 mg, 0.71 mmol) solution in dichloromethane (3.8 mL). The resulting mixture was stirred at r.t. for 5 min. To the reaction mixture, 2-((4-methoxybenzy)oxy) ethan-1-ol 7 (100 mg, 0.55 mmol) in dichloromethane (1.3 mL) was added at 0° C. The mixture was stirred at r.t. for 3 h. The reaction was quenched with Na₂SO₃ (aq) and NaHCO₃, then extracted with ethyl acetate. The organic phase were combined and evaporated to dryness. The crude material was purified by flush column chromatography to afford title compound. Yield: 131 mg (82%).

¹H NMR (400 MHz, CDCl₃) δ (ppm)=7.28 (2H, d, J=8.8 Hz), 6.89 (2H, d, J=8.8 Hz), 4.51 (2H, s), 3.81 (3H, s), 3.71 (2H, t, J=7.0 Hz), 3.26 (2H, t, J=6.7 Hz)

¹³C NMR (101 MHz, CDCl₃) δ (ppm)=159.4, 129.9, 129.4, 113.9, 72.6, 70.5, 55.3, 3.0.

### 5-((2-((4-methoxybenzyl)oxy)ethoxy)methyl)-2,2,5-trimethyl-1,3-dioxane (9)

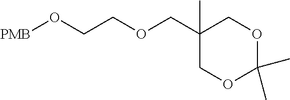

Sodium hydride 60% dispersion in mineral oil (384 mg, 9.60 mmol) was added to a solution of (2,2,5-trimethyl-1,3-dioxan-5-yl) methanol 1 (1.54 g, 9.60 mmol) in DMF (3.0 mL) at 0° C. The resulting mixture was stirred at r.t. for 30 min. To the mixture, 1-((2-iodoethoxy)methyl)-4-methoxybenzene 8 (700 mg, 2.40 mmol) in DMF (0.5 mL) was added dropwise at 0° C. The reaction mixture was stirred at 130° C. for 45 min. The mixture was quenched with H₂O and extracted with ethyl acetate. The organic phase was evaporated to dryness. The crude material was purified by flush column chromatography to afford title compound. Yield: 110 mg (14%).

¹H NMR (400 MHz, CDCl₃) δ (ppm)=7.29 (2H, d, J=6.3 Hz), 6.90 (2H, d, J=8.8 Hz), 4.53 (2H, s), 3.83 (3H, s), 3.74 (2H, d, J=11.9 Hz), 3.68-3.60 (4H, m), 3.56 (2H, d, J=11.7 Hz), 3.49 (2H, s), 1.45 (3H, s), 1.42 (3H, s), 0.92 (3H, s).

¹³C NMR (101 MHz, CDCl₃) δ (ppm)=159.3, 130.7, 129.4, 113.9, 98.0, 74.3, 73.0, 71.3, 69.2, 66.7, 55.4, 34.6, 26.4, 21.5, 18.5.

### 2-((2-((4-methoxybenzyl)oxy)ethoxy)methyl)-2-methylpropane-1,3-diol (10)

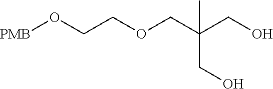

Trifluoroacetic acid (11 μL, 0.14 mmol) was added to 5-((2-((4-methoxybenzyl)oxy)ethoxy)methyl)-2,2,5-trimethyl-1,3-dioxane 9 (77 mg, 0.24 mmol) in methanol (0.6 mL) and H₂O (0.02 mL). The resulting mixture was stirred at r.t. for 16 h and then evaporated to dryness. The crude material was purified by flush column chromatography to afford title compound. Yield: 60 mg (89%).

¹H NMR (500 MHz, CDCl₃) δ (ppm)=7.26 (2H, d, J=8.5 Hz), 6.88 (2H, d, J=8.5 Hz), 4.49 (2H, s), 3.80 (3H, s), 3.51 (2H, s), 2.66 (2H, s), 0.80 (3H, s)

¹³C NMR (126 MHz, CDCl₃) δ (ppm)=159.5, 130.1, 129.6, 114.0, 73.1, 70.9, 69.0, 68.3, 55.4, 41.0, 17.4.

### 1,21-diazido-11-((2-((4-methoxybenzyl)oxy)ethoxy)methyl)-11-methyl-3,6,9,13,16,19-hexaoxahenicosane (11)

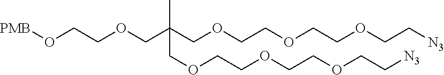

To a mixture of 2-((2-((4-methoxybenzyl)oxy)ethoxy)methyl)-2-methylpropane-1,3-diol 10 (73 mg, 0.26 mmol) and 2-(2-(2-azidoethoxy)ethoxy)ethyl methanesulfonate 3 (390 mg, 1.54 mmol) in 1,4-dioxane (0.51 mL), were added TBAB (25 mg, 0.077 mmol), potassium iodide (2.1 mg, 0.013 mmol) and potassium hydroxide powder (86 mg, 1.54 mmol). The resulting reaction mixture was stirred at 100° C. for 40 h. The reaction mixture was purified by flush column chromatography to afford title compound. Yield: 85 mg (55%).

¹H NMR (400 MHz, CDCl₃) δ (ppm)=7.26 (2H, d, J=8.4 Hz), 6.87 (2H, d, J=8.4 Hz), 4.49 (2H, s), 3.80 (3H, s), 3.70-3.53 (24H, m), 3.41-3.35 (4H, m), 3.35-3.30 (6H, m), 0.94 (3H, s)

¹³C NMR (101 MHz, CDCl₃) δ (ppm)=159.3, 130.8, 129.3, 113.9, 74.1, 72.9, 71.2, 70.9, 70.8, 70.7, 70.2, 69.3, 55.4, 50.9, 41.2, 17.5.

### 1-azido-11-((2-(2-(2-azidoethoxy)ethoxy)ethoxy)methyl)-11-methyl-3,6,9,13-tetraoxapentadecan-15-ol (12)

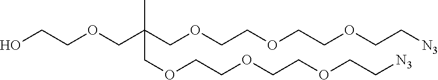 [SMILES: CC(COCCO)(COCCOCCOCCN=[N+]=[N-])COCCOCCOCCN=[N+]=[N-]]

To a mixture of 1,21-diazido-11-((2-((4-methoxybenzyl)oxy)ethoxy)methyl)-11-methyl-3,6,9,13,16,19-hexaoxahenicosane 11 (118 mg, 0.20 mmol) in H₂O (0.20 mL) and dichloromethane (2.0 mL), 2,3-dichloro-5,6-dicyano-1,4-benzoquinone (49.2 mg, 0.22 mmol) was added at 0° C. The resulting reaction mixture was stirred at 4° C. for 16 h. The reaction mixture was quenched with NaHCO₃ (aq) and filtered to remove precipitate. The filtrate was evaporated and the remaining residue was purified by HPLC under acidic condition (5-95% CH₃CN in 0.1% aq. HCO₂H) to afford title compound. Yield: 86 mg (91%).

¹H NMR (500 MHz, CDCl₃) δ (ppm)=3.71-3.61 (18H, m), 3.60-3.55 (4H, m), 3.55-3.51 (2H, m), 3.42-3.30 (10H, m), 2.48 (1H, t, J=6.2 Hz), 0.94 (3H, s).

¹³C NMR (126 MHz, CDCl₃) δ (ppm)=74.2, 73.5, 72.3, 71.2, 70.9, 70.8, 70.7, 70.2, 61.7, 50.9, 41.1, 17.7.

### 11-((2-(allyloxy)ethoxy)methyl)-1,21-diazido-11-methyl-3,6,9,13,16,19-hexaoxahenicosane (13)

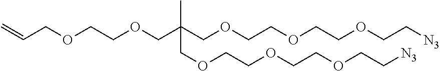 [SMILES: C=CCOCCOCC(C)(COCCOCCOCCN=[N+]=[N-])COCCOCCOCCN=[N+]=[N-]]

To a mixture of 1-azido-11-((2-(2-(2-azidoethoxy)ethoxy)ethoxy)methyl)-11-methyl-3,6,9,13-tetraoxapentadecan-15-ol 12 (50 mg, 0.10 mmol) in 1,4-dioxane (0.21 mL), were added allyl bromide (26 mg, 0.31 mmol), and potassium hydroxide powder (18 mg, 0.31 mmol). The resulting reaction mixture was stirred at 80° C. for 6 h. The reaction mixture was diluted with dichloromethane and evaporated. The remaining residue was purified by flush column chromatography to afford title compound. Yield: 39 mg (72%).

¹H NMR (500 MHz, CDCl₃) δ (ppm)=5.95-5.85 (1H, m), 5.26 (1H, dd, J=1.7, 17.2 Hz), 5.16 (1H, dd, J=1.4, 10.5 Hz), 4.01 (2H, d, J=6.0 Hz), 3.73-3.59 (16H, m), 3.58-3.52 (8H, m), 3.40-3.35 (4H, m), 3.35-3.28 (6H, m), 0.93 (3H, s)

¹³C NMR (126 MHz, CDCl₃) δ (ppm)=135.1, 116.8, 74.1, 72.2, 71.2, 70.9, 70.8, 70.7, 70.2, 69.5, 50.8, 41.2, 17.5.

### 1-azido-11-((2-(2-(2-azidoethoxy)ethoxy)ethoxy)methyl)-11-methyl-3,6,9,13-tetraoxapentadecan-15-al (14)

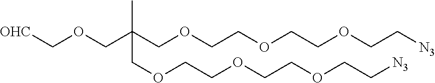 [SMILES: CC(COCC=O)(COCCOCCOCCN=[N+]=[N-])COCCOCCOCCN=[N+]=[N-]]

To a mixture of 11-((allyloxy)methyl)-1,21-diazido-11-methyl-3,6,9,13,16,19-hexaoxahenicosane 5 (25 mg, 0.053 mmol) in H₂O (0.3 mL) and 1,4-dioxane (1.0 mL), were added 2,6-luthidine (12.2 μL, 0.11 mmol), osmium tetroxide 4% in H₂O (6.7 μL, 0.0011 mmol), sodium periodate (45 mg, 0.21 mmol). The resulting reaction mixture was stirred at r.t. for 16 h. The reaction mixture was quenched with Na₂SO₃ (aq) and extracted with dichloromethane. The organic layer was concentrated and the remaining residue was purified by flush column chromatography to afford title compound. Yield: 16 mg (64%).

¹H NMR (500 MHz, CDCl₃) δ (ppm)=9.73 (1H, s), 4.02 (2H, s), 3.74-3.52 (20H, m), 3.46-3.26 (10H, m), 0.98 (3H, s).

¹³C NMR (126 MHz, CDCl₃) δ (ppm)=202.2, 77.0, 74.7, 73.9, 71.2, 70.9, 70.8, 70.7, 70.2, 50.9, 41.3, 17.5.

### 1-azido-11-((2-(2-(2-azidoethoxy)ethoxy)ethoxy)methyl)-11-methyl-3,6,9,13,16-pentaoxaoctadecan-18-al (15)

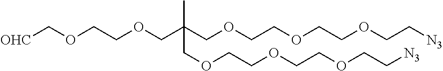 [SMILES: CC(COCCOCC=O)(COCCOCCOCCN=[N+]=[N-])COCCOCCOCCN=[N+]=[N-]]

To a mixture of 11-((2-(allyloxy)ethoxy)methyl)-1,21-diazido-11-methyl-3,6,9,13,16,19-hexaoxahenicosane 13 (50 mg, 0.096 mmol) in H₂O (0.6 mL) and 1,4-dioxane (1.7 mL), were added 2,6-luthidine (22.4 μL, 0.19 mmol), osmium tetroxide 4% in H₂O (12.2 μL, 0.0019 mmol) and sodium periodate (82.5 mg, 0.39 mmol). The resulting reaction mixture was stirred at r.t. for 16 h. The reaction mixture was quenched with Na₂SO₃ (aq) and extracted with dichloromethane. The organic layer was concentrated and the remaining residue was purified by flush column chromatography to afford title compound. Yield: 38 mg (76%).

¹H NMR (400 MHz, CDCl₃) δ (ppm)=9.71 (1H, s), 4.14 (2H, s), 3.71-3.50 (24H, m), 3.44-3.25 (10H, m), 0.92 (3H, s)

¹³C NMR (101 MHz, CDCl₃) δ (ppm)=201.3, 77.0, 73.9, 73.4, 71.1, 70.8, 70.6, 70.1, 55.1, 50.8, 41.0, 17.5

### 1-azido-14-(13-azido-2,5,8,11-tetraoxatridecyl)-14-methyl-3,6,9,12,16-pentaoxaoctadecan-18-al (16)

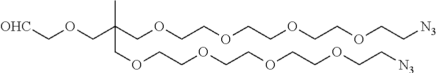 [SMILES: CC(COCC=O)(COCCOCCOCCOCCN=[N+]=[N-])COCCOCCOCCOCCN=[N+]=[N-]]

To a mixture of 14-((allyloxy)methyl)-1,27-diazido-14-methyl-3,6,9,12,16,19,22,25-octaoxaheptacosane 6 (35 mg, 0.062 mmol) in H₂O (0.4 mL) and 1,4-dioxane (1.7 mL) were added 2,6-luthidine (14.5 μL, 0.12 mmol), osmium tetroxide 4% in H₂O (12.2 μL, 0.0012 mmol), sodium periodate (53 mg, 0.25 mmol). The resulting reaction mixture was stirred at r.t. for 16 h. The reaction mixture was quenched with Na₂SO₃ (aq) and extracted with dichloromethane. The organic layer was concentrated and the remaining residue was purified by flush column chromatography to afford title compound. Yield: 23 mg (65%).

¹H NMR (500 MHz, CDCl₃) δ (ppm)=9.75 (1H, s), 4.10-3.99 (2H, m), 3.77-3.29 (38H, m), 1.00 (3H, s).

¹³C NMR (126 MHz, CDCl₃) δ (ppm)=202.2, 74.7, 73.9, 71.2, 70.9, 70.8, 70.6, 70.2, 50.9, 41.3, 17.5.

### 1-azido-11-((2-(2-(2-azidoethoxy)ethoxy)ethoxy)methyl)-11-methyl-3,6,9,13-tetraoxapentadecan-15-oic acid (17)

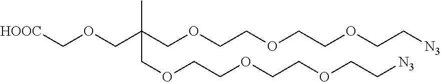 [SMILES: CC(COCCOCCOCCN=[N+]=[N-])(COCCOCCOCCN=[N+]=[N-])COCC(=O)O]

To a mixture of 1-azido-11-((2-(2-(2-azidoethoxy)ethoxy)ethoxy)methyl)-11-methyl-3,6,9,13-tetraoxapentadecan-15-al 14 (16 mg, 0.034 mmol) in t-BuOH (0.6 mL) were added 2M 2-methyl-2-butene in THE (84 μL, 0.168 mmol), NaH₂PO₄ (4.0 mg, 0.034 mmol), sodium chlorite (12.1 mg, 0.134 mmol) in H₂O (0.2 mL). The resulting reaction mixture was stirred at r.t. for 16 h. The reaction mixture was concentrated and then diluted with NaOH (aq). The mixture was washed with MTBE and neutralized by 2M HCl. Extracted with dichloromethane, the organic layer was dried by Na₂SO₄ and concentrated. The remaining crude was used in next step without further purification. Yield: 16 mg (97%).

¹H NMR (500 MHz, CDCl₃) δ (ppm)=4.05 (2H, s), 3.70-3.58 (20H, m), 3.45-3.33 (10H, m), 0.95 (3H, s).

¹³C NMR (126 MHz, CDCl₃) δ (ppm)=172.1, 75.4, 74.7, 71.3, 70.9, 70.7, 70.4, 70.2, 68.8, 50.8, 40.8, 18.0.

### 1-azido-11-((2-(2-(2-azidoethoxy)ethoxy)ethoxy)methyl)-11-methyl-3,6,9,13,16-pentaoxaoctadecan-18-oic acid (18)

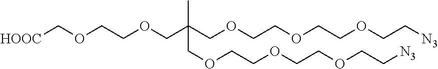 [SMILES: CC(COCCOCCOCCN=[N+]=[N-])(COCCOCCOCCN=[N+]=[N-])COCCOCC(=O)O]

To a mixture of 1-azido-11-((2-(2-(2-azidoethoxy)ethoxy)ethoxy)methyl)-11-methyl-3,6,9,13,16-pentaoxaoctadecan-18-al 15 (35 mg, 0.067 mmol) in t-butanol (1.2 mL), were added 2M 2-methyl-2-butene in THE (168 μL, 0.336 mmol), NaH₂PO₄ (8.1 mg, 0.067 mmol), sodium chlorite (24 mg, 0.265 mmol) in H₂O (0.4 mL). The resulting reaction mixture was stirred at r.t. for 16 h. The reaction mixture was concentrated and then diluted with NaOH (aq). The mixture was washed with MTBE and neutralized with 2M HCl. Extracted with dichloromethane, the organic layer was dried by Na₂SO₄ and concentrated. The remaining crude was used in next step without further purification. Yield: 36 mg (quant.).

¹H NMR (500 MHz, CDCl₃) δ (ppm)=4.09 (2H, s), 3.71-3.47 (24H, m), 3.37-3.21 (10H, m), 0.87 (3H, s)

¹³C NMR (126 MHz, CDCl₃) δ (ppm)=172.2, 74.0, 73.9, 71.5, 71.1, 70.8, 70.7, 70.1, 68.9, 50.8, 41.0, 17.5.

### 1-azido-14-(13-azido-2,5,8,11-tetraoxatridecyl)-14-methyl-3,6,9,12,16-pentaoxaoctadecan-18-oic acid (19)

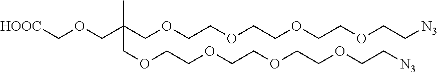 [SMILES: CC(COCCOCCOCCOCCN=[N+]=[N-])(COCCOCCOCCOCCN=[N+]=[N-])COCC(=O)O]

To a mixture of 1-azido-14-(13-azido-2,5,8,11-tetraoxatridecyl)-14-methyl-3,6,9,12,16-pentaoxaoctadecan-18-al 16 (14 mg, 0.025 mmol) in t-Butanol (0.45 mL), were added 2M 2-methyl-2-butene in THF (62 μL, 0.124 mmol), NaH₂PO₄ (3.0 mg, 0.025 mmol), sodium chlorite (8.9 mg, 0.099 mmol) in H₂O (0.15 mL). The resulting reaction mixture was stirred at r.t. for 16 h. The reaction mixture was concentrated and then diluted with NaOH (aq). The mixture was washed with MTBE and neutralized by 2M HCl. Extracted with dichloromethane, the organic layer was dried by Na₂SO₄ and concentrated. The remaining crude was used in next step without further purification. Yield: 8 mg (56%).

¹H NMR (500 MHz, CDCl₃) δ (ppm)=4.04 (2H, s), 3.71-3.55 (28H, m), 3.45-3.32 (10H, m), 0.95 (3H, s).

¹³C NMR (126 MHz, CDCl₃) δ (ppm)=171.9, 75.3, 74.7, 71.4, 70.9, 70.8, 70.7, 70.4, 70.2, 68.9, 50.9, 40.8, 18.0.

### (2S,4R)-1-((S)-1-azido-11-((2-(2-(2-azidoethoxy)ethoxy)ethoxy)methyl)-17-(tert-butyl)-11-methyl-15-oxo-3,6,9,13-tetraoxa-16-azaoctadecan-18-oyl)-4-hydroxy-N-(4-(4-methylthiazol-5-yl)benzyl)pyrrolidine-2-carboxamide (20)

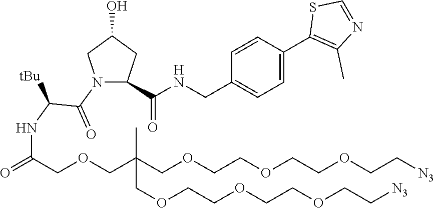 [SMILES: Cc1ncsc1-c1ccc(CNC(=O)[C@@H]2C[C@@H](O)CN2C(=O)[C@@H](NC(=O)COCC(C)(COCCOCCOCCN=[N+]=[N-])COCCOCCOCCN=[N+]=[N-])C(C)(C)C)cc1]

COMU (10.4 mg, 0.024 mmol), N,N-diisopropylethylamine (14.1 μL, 0.081 mmol) were added to a mixture of 1-azido-11-((2-(2-(2-azidoethoxy)ethoxy)ethoxy)methyl)-11-methyl-3,6,9,13-tetraoxapentadecan-15-oic acid 17 (10 mg, 0.020 mmol) in DMF (0.20 mL). The resulting reaction mixture was stirred at r.t. for 2 min. VH032 amine hydrochloride 53 (14.2 mg, 0.031 mmol) was added to the mixture. Then, the mixture was stirred at r.t. for 16 h and purified by HPLC under acidic condition (5-95% CH₃CN in 0.1% aq. HCO₂H) to afford title compound. Yield: 10 mg (54%).

¹H NMR (500 MHz, CDCl₃) δ (ppm)=8.68 (1H, s), 7.39-7.30 (5H, m), 7.10 (1H, d, J=8.5 Hz), 4.73 (1H, t, J=7.8 Hz), 4.59-4.51 (2H, m), 4.48 (1H, d, J=8.7 Hz), 4.35 (1H, dd, J=5.5, 15.0 Hz), 4.09 (1H, d, J=12.0 Hz), 3.94 (2H, dd, J=15.4, 17.7 Hz), 3.71-3.53 (21H, m), 3.46-3.30 (10H, m), 2.60-2.49 (1H, m), 2.51 (3H, s), 2.16-2.08 (1H, m), 0.96 (3H, s), 0.95 (9H, s).

¹³C NMR (126 MHz, CDCl₃) δ (ppm)=171.5, 170.8, 170.7, 150.5, 148.6, 138.3, 131.8, 131.1, 129.7, 128.3, 74.8, 74.2, 74.1, 71.2, 70.9, 70.8, 70.7, 70.6, 70.3, 70.2, 58.5, 57.2, 56.7, 50.8, 43.4, 41.1, 35.9, 35.0, 26.5, 17.7, 16.2.

MS (ESI) for C₄₁H₆₅N₁₀O₁₁S [M+H⁺] calculated 905.5, obtained 905.3.

### (2S,4R)-1-((S)-21-azido-11-((2-(2-(2-azidoethoxy)ethoxy)ethoxy)methyl)-2-(tert-butyl)-11-methyl-4-oxo-6,9,13,16,19-pentaoxa-3-azahenicosanoyl)-4-hydroxy-N-(4-(4-methylthiazol-5-yl)benzyl)pyrrolidine-2-carboxamide (21)

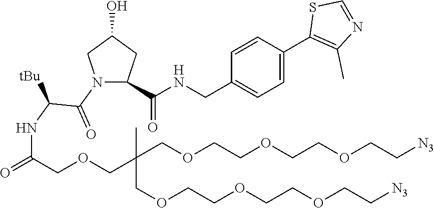 [SMILES: Cc1ncsc1-c1ccc(CNC(=O)[C@@H]2C[C@@H](O)CN2C(=O)[C@@H](NC(=O)COCC(C)(COCCOCCOCCN=[N+]=[N-])COCCOCCOCCN=[N+]=[N-])C(C)(C)C)cc1]

To a mixture of 1-azido-11-((2-(2-(2-azidoethoxy)ethoxy)ethoxy)methyl)-11-methyl-3,6,9,13,16-pentaoxaoctadecan-18-oic acid 18 (17 mg, 0.032 mmol) in DMF (0.32 mL), were added HATU (18 mg, 0.048 mmol), HOAt (6.5 mg, 0.048 mmol), N,N-diisopropylethylamine (22 μL, 0.127 mmol). The resulting reaction mixture was stirred at r.t. for 5 min. VH032 amine hydrochloride 53 (22.1 mg, 0.032 mmol) was added to the mixture. The mixture was then stirred at r.t. for 6 h and purified by HPLC under acidic condition (5-95% CH₃CN in 0.1% aq. HCO₂H) to afford title compound. Yield: 13 mg (43%).

¹H NMR (500 MHz, CDCl₃) δ (ppm)=8.75 (1H, s), 7.44-7.34 (5H, m), 7.21 (1H, d, J=8.0 Hz), 4.76 (1H, t, J=7.8 Hz), 4.61-4.50 (3H, m), 4.38 (1H, dd, J=5.4, 14.8 Hz), 4.12-3.97 (3H, m), 3.74-3.55 (25H, m), 3.43-3.29 (10H, m), 2.61-2.51 (1H, m), 2.54 (3H, s), 2.17-2.10 (1H, m), 0.97 (9H, s), 0.94 (3H, s).

¹³C NMR (126 MHz, CDCl₃) δ (ppm)=171.6, 170.8, 170.5, 150.6, 148.3, 138.4, 131.9, 130.9, 129.7, 128.3, 74.2, 74.1, 71.2, 71.1, 70.9, 70.8, 70.6, 70.3, 70.2, 58.6, 57.2, 56.8, 50.9, 43.4, 41.1, 36.0, 35.1, 26.6, 17.5, 16.0.

MS (ESI) for C₄₃H₆₉N₁₀O_{12S} [M+H⁺] calculated 949.5, obtained 949.4.

### (2S,4R)-1-((S)-1-azido-14-(13-azido-2,5,8,11-tetraoxatridecyl)-20-(tert-butyl)-14-methyl-18-oxo-3,6,9,12,16-pentaoxa-19-azahenicosan-21-oyl)-4-hydroxy-N-(4-(4-methylthiazol-5-yl)benzyl)pyrrolidine-2-carboxamide (22)

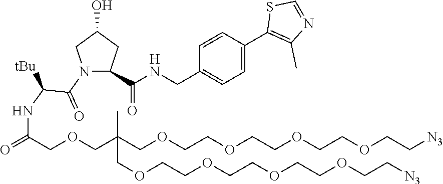 [SMILES: Cc1ncsc1-c1ccc(CNC(=O)[C@@H]2C[C@@H](O)CN2C(=O)[C@@H](NC(=O)COCC(C)(COCCOCCOCCOCCN=[N+]=[N-])COCCOCCOCCOCCN=[N+]=[N-])C(C)(C)C)cc1]

To a mixture of 1-azido-14-(13-azido-2,5,8,11-tetraoxatridecyl)-14-methyl-3,6,9,12,16-pentaoxaoctadecan-18-oic acid 19 (7.1 mg, 0.012 mmol) in DMF (0.20 mL), were added HATU (7.0 mg, 0.018 mmol), HOAt (2.5 mg, 0.018 mmol), N,N-diisopropylethylamine (8.5 μL, 0.127 mmol). The resulting reaction mixture was stirred at r.t. for 5 min. VH032 amine hydrochloride 53 (22.1 mg, 0.049 mmol) was added to the mixture. Then, the mixture was stirred at r.t. for 6 h and purified by HPLC under acidic condition (5-95% CH₃CN in 0.1% aq. HCO₂H) to afford title compound. Yield: 6.2 mg (51%).

¹H NMR (500 MHz, CD₃OD) δ (ppm)=8.90 (1H, s), 7.54-7.36 (5H, m), 4.69 (1H, d, J=9.7 Hz), 4.61-4.48 (3H, m), 4.36 (1H, dd, J=5.1, 15.6 Hz), 4.00 (1H, d, J=14.8 Hz), 3.96 (1H, d, J=15.4 Hz), 3.87 (1H, d, J=11.5 Hz), 3.80 (1H, dd, J=3.7, 10.8 Hz), 3.69-3.55 (28H, m), 3.48 (1H, d, J=9.3 Hz), 3.44 (1H, d, J=9.0 Hz), 3.41-3.34 (8H, m), 2.49 (3H, s), 2.27-2.19 (1H, m), 2.14-2.06 (1H, m), 1.05 (9H, s), 1.01 (3H, s)

¹³C NMR (126 MHz, CDCl₃) δ (ppm)=174.4, 171.9, 171.6, 152.9, 149.0, 140.3, 133.5, 131.5, 130.4, 129.0, 75.5, 74.8, 74.8, 72.2, 71.7, 71.6, 71.5, 71.1, 60.8, 58.1, 51.8, 43.7, 42.1, 38.9, 37.3, 27.0, 18.0, 15.8.

MS (ESI) for C₄₅H₇₃N₁₀O₁₃S [M+H⁺] calculated 993.5, obtained 993.4.

### N,N′-(11-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (SIM1)

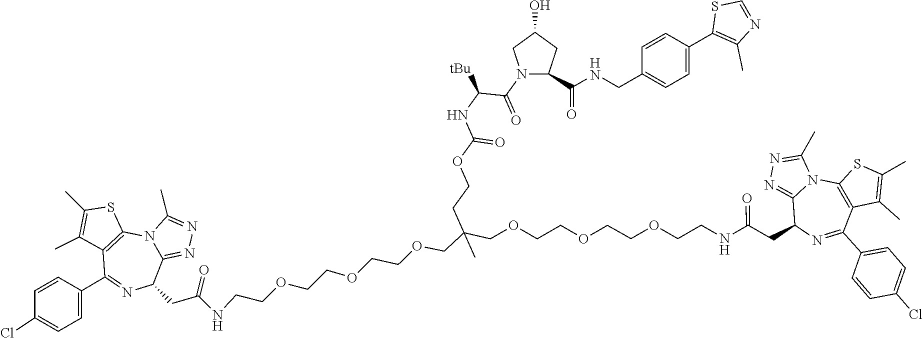 [SMILES: Cc1ncsc1-c1ccc(CNC(=O)[C@@H]2C[C@@H](O)CN2C(=O)[C@@H](NC(=O)OCCC(C)(COCCOCCOCCNC(=O)C[C@@H]2N=C(c3ccc(Cl)cc3)c3c(sc(C)c3C)-n3c(C)nnc32)COCCOCCOCCNC(=O)C[C@@H]2N=C(c3ccc(Cl)cc3)c3c(sc(C)c3C)-n3c(C)nnc32)C(C)(C)C)cc1]

To a mixture of (2S,4R)-1-((S)-1-azido-11-((2-(2-(2-azidoethoxy)ethoxy)ethoxy)methyl)-17-(tert-butyl)-11-methyl-15-oxo-3,6,9,13-tetraoxa-16-azaoctadecan-18-oyl)-4-hydroxy-N-(4-(4-methylthiazol-5-yl)benzyl)pyrrolidine-2-carboxamide 20 (10 mg, 0.011 mmol) in MeOH (0.60 mL), were added 10% wt palladium on carbon (2.0 mg). The resulting reaction mixture was stirred under hydrogen atmosphere at r.t. for 2 h. The mixture was then filtered on a celite pad and evaporated. A pre stirred mixture of (+)-JQ1 carboxylic acid (12.5 mg, 0.031 mmol), COMU (13.4 mg, 0.031 mmol), N,N-diisopropylethylamine (13.6 μL, 0.078 mmol) in DMF (0.20 mL) was added to the concentrated crude. Then, the mixture was stirred at r.t. for 3 h and purified by HPLC under acidic condition (5-95% CH₃CN in 0.1% aq. HCO₂H) to afford title compound. Yield: 10 mg (54%).

¹H NMR (500 MHz, CDCl₃) δ (ppm)=8.67 (1H, s), 7.47-7.28 (14H, m), 7.17 (1H, d, J=9.7 Hz), 4.86 (1H, m), 4.83 (t, 1H, J=7.9 Hz), 4.70-4.61 (3H, m), 4.53 (1H, dd, J=5.7, 15.2 Hz), 4.48-4.42 (1H, m), 4.34 (1H, dd, J=6.0, 14.9 Hz), 4.1 (1H, d, J=11.1 Hz), 4.06 (1H, d, J=15.3 Hz), 3.96 (1H, d, J=15.3 Hz), 3.70-3.24 (31H, m), 3.21 (1H, d, J=8.9 Hz), 2.63 (6H, s), 2.50 (3H, s), 2.44 (1H, m), 2.39 (6H, s), 2.15 (1H, m), 1.94-1.84 (4H, m), 1.65 (6H, s), 0.97 (9H, s), 0.93 (3H, s).

¹³C NMR (126 MHz, CDCl₃) δ (ppm)=171.5, 171.2, 170.8, 170.7, 164.0, 155.9, 150.4, 149.9, 148.6, 138.5, 136.9, 132.3, 131.1, 130.9, 130.7, 130.1, 129.6, 128.8, 128.2, 73.7, 73.6, 73.5, 71.2, 70.8, 70.7, 70.6, 70.4, 70.3, 70.1, 59.0, 57.3, 56.7, 54.5, 43.3, 41.1, 39.6, 38.8, 36.5, 35.6, 26.6, 17.7, 16.2, 14.5.

HRMS (ESI) for C₇₉H₉₉Cl₂N₁₄O₁₃S₃ [M+H⁺] calculated 1617.6050, obtained 1617.6390.

### N,N′-(11-((2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (SIM2)

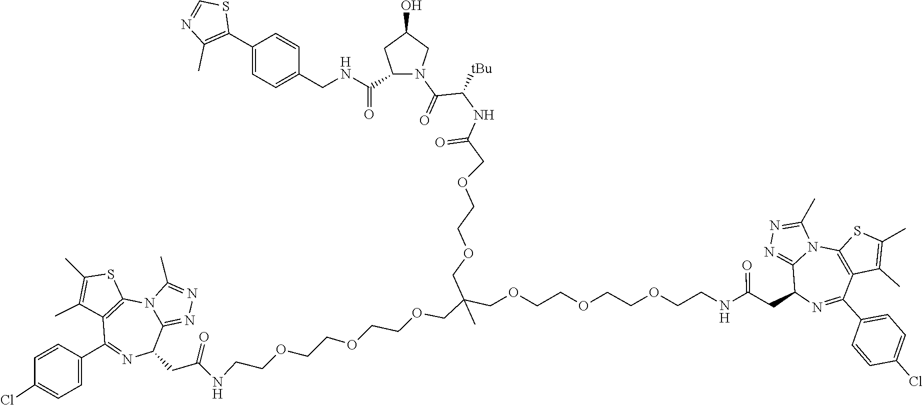 [SMILES: Cc1ncsc1-c1ccc(CNC(=O)[C@@H]2C[C@@H](O)CN2C(=O)[C@@H](NC(=O)COCCOCC(C)(COCCOCCOCCNC(=O)C[C@@H]2N=C(c3ccc(Cl)cc3)c3c(sc(C)c3C)-n3c(C)nnc32)COCCOCCOCCNC(=O)C[C@@H]2N=C(c3ccc(Cl)cc3)c3c(sc(C)c3C)-n3c(C)nnc32)C(C)(C)C)cc1]

To a mixture of (2S,4R)-1-((S)-21-azido-11-((2-(2-(2-azidoethoxy)ethoxy)ethoxy)methyl)-2-(tert-butyl)-11-methyl-4-oxo-6,9,13,16,19-pentaoxa-3-azahenicosanoyl)-4-hydroxy-N-(4-(4-methylthiazol-5-yl)benzyl)pyrrolidine-2-carboxamide 21 (13 mg, 0.014 mmol) in MeOH (0.80 mL), were added 10% wt palladium on carbon (2.5 mg). The resulting reaction mixture was stirred under hydrogen atmosphere at r.t. for 2 h. The mixture was then filtered on a celite pad and evaporated. A pre stirred mixture of (+)-JQ1 carboxylic acid (22.2 mg, 0.055 mmol), COMU (23.7 mg, 0.055 mmol), N,N-diisopropylethylamine (16 μL, 0.092 mmol) in DMF (0.20 mL) was added to the concentrated crude. Then, the mixture was stirred at r.t. for 16 h and purified by HPLC under acidic condition (5-95% CH₃CN in 0.1% aq. HCO₂H) to afford title compound. Yield: 12 mg (53%).

¹H NMR (500 MHz, CDCl₃) δ (ppm)=8.67 (1H, s), 7.57 (1H, t, J=4.9 Hz), 7.42-7.28 (12H, m), 7.25-7.15 (2H, m), 4.81 (1H, t, J=7.7 Hz), 4.69-4.60 (3H, m), 4.54-4.47 (2H, m), 4.46-4.42 (1H, m), 4.36 (1H, dd, J=5.8, 15.3 Hz), 4.07 (1H, d, J=15.4 Hz), 4.05 (1H, br d, J=11.1 Hz), 3.98 (1H, d, J=15.4 Hz), 3.73-3.23 (36H, m), 2.63 (3H, s), 2.62 (3H, s), 2.50 (3H, s), 2.47-2.40 (1H, m), 2.39 (6H, s), 2.23-2.16 (1H, m), 2.01-1.91 (4H, m), 1.66 (6H, s), 0.97 (9H, s), 0.92 (3H, s).

¹³C NMR (126 MHz, CDCl₃) δ (ppm)=171.4, 171.2, 170.7, 170.2, 163.9, 155.9, 150.3, 149.9, 148.6, 138.5, 136.9, 136.8, 132.3, 131.8, 131.1, 130.9, 130.7, 130.0, 129.6, 128.8, 128.2, 74.0, 73.8, 71.2, 71.1, 71.0, 70.7, 70.6, 70.1, 59.0, 57.2, 56.8, 54.5, 43.3, 41.2, 39.6, 39.0, 36.5, 35.8, 26.6, 17.6, 16.2, 14.5, 13.2, 11.9.

HRMS (ESI) for C₈₁H₁₀₃Cl₂N₁₄O₁₄S₃[M+H⁺] calculated 1661.6312, obtained 1661.8200.

### N,N′-(14-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-14-methyl-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (SIM3)

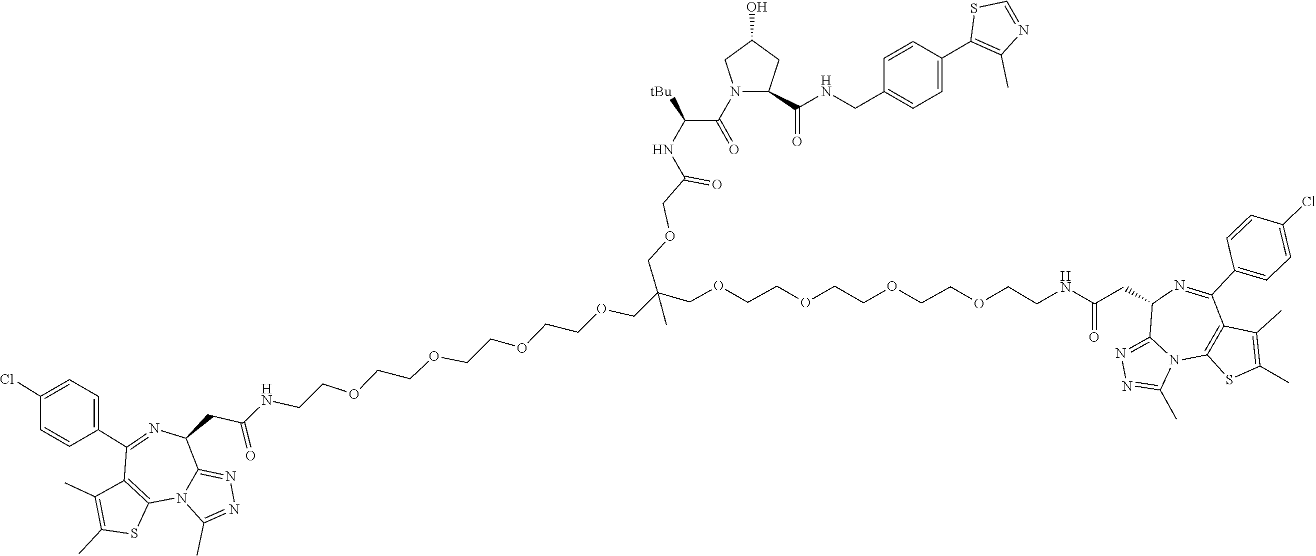 [SMILES: Cc1ncsc1-c1ccc(CNC(=O)[C@@H]2C[C@@H](O)CN2C(=O)[C@@H](NC(=O)COCC(C)(COCCOCCOCCOCCNC(=O)C[C@@H]2N=C(c3ccc(Cl)cc3)c3c(sc(C)c3C)-n3c(C)nnc32)COCCOCCOCCOCCNC(=O)C[C@@H]2N=C(c3ccc(Cl)cc3)c3c(sc(C)c3C)-n3c(C)nnc32)C(C)(C)C)cc1]

To a mixture of (2S,4R)-1-((S)-1-azido-14-(13-azido-2,5,8,11-tetraoxatridecyl)-20-(tert-butyl)-14-methyl-18-oxo-3,6,9,12,16-pentaoxa-19-azahenicosan-21-oyl)-4-hydroxy-N-(4-(4-methylthiazol-5-yl)benzyl)pyrrolidine-2-carboxamide 22 (10 mg, 0.020 mmol) in MeOH (0.20 mL), were added 10% wt palladium on carbon (10.4 mg). The resulting reaction mixture was stirred under hydrogen atmosphere at r.t. for 2 h. The mixture was then filtered on a celite pad and evaporated. A pre stirred mixture of (+)-JQ1 carboxylic acid (14.8 mg, 0.037 mmol), HATU (14 mg, 0.037 mmol), HOAt (5.0 mg, 0.037 mmol), N,N-diisopropylethylamine (12.8 μL, 0.074 mmol) in DMF (0.12 mL) was added to the concentrated crude. Then, the mixture was stirred at r.t. for 16 h and purified by HPLC under acidic condition (5-95% CH₃CN in 0.1% aq. HCO₂H) to afford title compound. Yield: 13 mg (43%).

¹H NMR (500 MHz, CD₃OD) δ (ppm)=8.86 (1H, s), 7.52-7.36 (12H, m), 4.71-4.40 (6H, m), 4.39-4.29 (1H, m), 3.97 (1H, d, J=14.4 Hz), 3.93 (1H, d, J=15.6 Hz), 3.85 (1H, d, J=10.8 Hz), 3.79 (1H, dd, J=3.9, 11.1 Hz), 3.68-3.33 (41H, m), 3.28 (1H, d, J=5.1 Hz), 2.68 (6H, s), 2.46 (3H, s), 2.44 (6H, s), 2.26-2.18 (1H, m), 2.12-2.05 (1H, m), 1.69 (6H, s), 1.03 (9H, s), 0.97 (3H, s).

¹³C NMR (126 MHz, CD₃OD) δ (ppm)=174.4, 172.9, 171.8, 171.6, 166.1, 157.1, 152.8, 149.1, 140.3, 138.2, 137.9, 133.5, 133.2, 132.0, 131.5, 131.4, 130.5, 130.4, 129.8, 129.0, 75.5, 74.8, 72.2, 71.7, 71.6, 71.5, 71.4, 71.1, 70.7, 60.9, 58.1, 58.0, 55.2, 43.7, 42.1, 40.6, 38.9, 38.8, 37.3, 27.0, 18.0, 15.9, 14.4, 12.9, 11.6.

HRMS (ESI) for C₈₃H₁₇Cl₂N₁₄O₁₅S₃ [M+H⁺] calculated 1705.6574, obtained 1705.6430.

### Synthesis of Compounds SIM4, SIM5

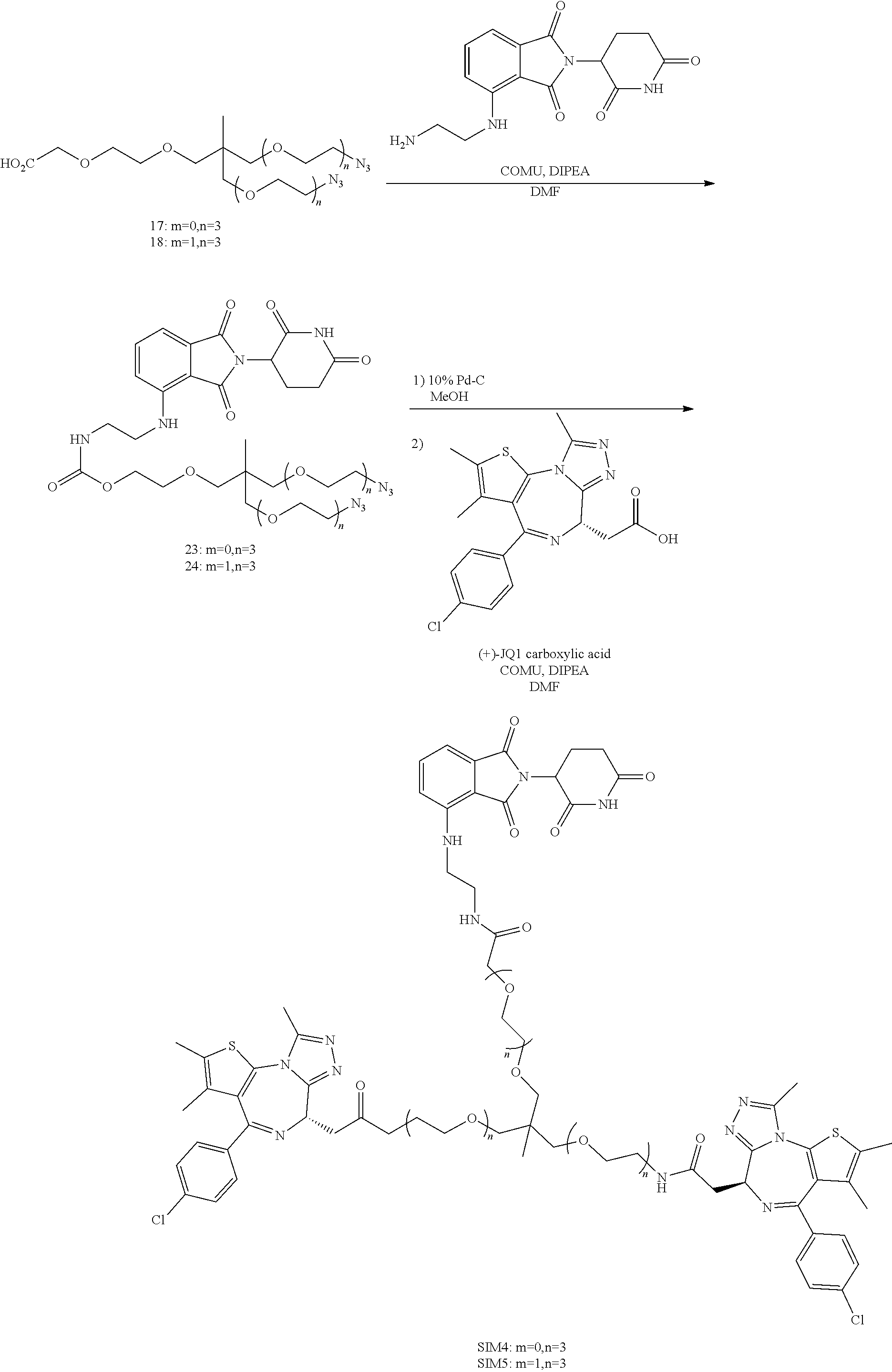

### 1-azido-11-((2-(2-(2-azidoethoxy)ethoxy)ethoxy)methyl)-N-(2-((2-(2,6-dioxopiperidin-3-yl)-1,3-dioxoisoindolin-4-yl)amino)ethyl)-11-methyl-3,6,9,13-tetraoxapentadecan-15-amide (23)

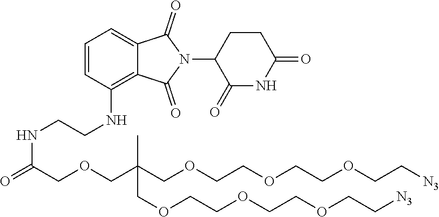 [SMILES: CC(COCCOCCOCCN=[N+]=[N-])(COCCOCCOCCN=[N+]=[N-])COCC(=O)NCCNc1cccc2c1C(=O)N(C1CCC(=O)NC1=O)C2=O]

To a mixture of 1-azido-11-((2-(2-(2-azidoethoxy)ethoxy)ethoxy)methyl)-11-methyl-3,6,9,13-tetraoxapentadecan-15-oic acid 17 (14 mg, 0.0284 mmol) in DMF (0.2 mL), were added COMU (13.4 mg, 0.031 mmol), N,N-diisopropylethylamine (14.8 μL, 0.085 mmol). The resulting reaction mixture was stirred at r.t. for 2 min. 4-[(2-aminoethyl)amino]-2-(2,6-dioxo-3-piperidinyl)-1H-isoindole-1,3(2H)-dione⁵⁴ (10.8 mg, 0.034 mmol) was added to the mixture. Then, the mixture was stirred at r.t. for 1 h and purified by HPLC under acidic condition (5-95% CH₃CN in 0.1% aq. HCO₂H) to afford title compound. Yield: 12.5 mg (56%).

¹H NMR (500 MHz, CDCl₃) δ (ppm)=8.27 (1H, s), 7.54 (1H, t, J=7.8 Hz), 7.14 (1H, d, J=7.1 Hz), 7.07 (1H, d, J=8.6 Hz), 6.50 (1H, t, J=5.7 Hz), 4.94 (1H, dd, J=5.3, 12.3 Hz), 4.00-3.95 (2H, m), 3.73-3.49 (24H, m), 3.45-3.32 (10H, m), 2.94-2.70 (3H, m), 2.21-2.11 (1H, m), 0.92 (3H, s).

¹³C NMR (126 MHz, CDCl₃) δ (ppm)=171.4, 171.1, 169.5, 168.4, 167.7, 147.0, 136.4, 132.7, 117.0, 112.0, 110.5, 75.0, 74.4, 71.2, 70.8, 70.7, 70.5, 70.1, 50.9, 49.1, 42.3, 40.9, 38.7, 31.6, 22.9.

MS (ESI) for C₃₄H₅₁N₁₀O₁₂ [M+H⁺] calculated 791.4, obtained 791.3.

### 1-azido-11-((2-(2-(2-azidoethoxy)ethoxy)ethoxy)methyl)-N-(2-((2-(2,6-dioxopiperidin-3-yl)-1,3-dioxoisoindolin-4-yl)amino)ethyl)-11-methyl-3,6,9,13,16-pentaoxaoctadecan-18-amide (24)

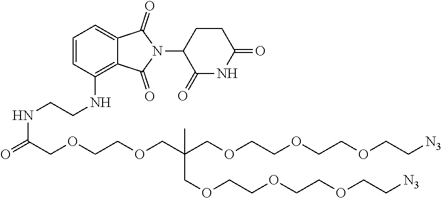 [SMILES: CC(COCCOCCOCCN=[N+]=[N-])(COCCOCCOCCN=[N+]=[N-])COCCOCC(=O)NCCNc1cccc2c1C(=O)N(C1CCC(=O)NC1=O)C2=O]

To a mixture of 1-azido-11-((2-(2-(2-azidoethoxy)ethoxy)ethoxy)methyl)-11-methyl-3,6,9,13,16-pentaoxaoctadecan-18-oic acid 18 (14 mg, 0.026 mmol) in DMF (0.42 mL), were added COMU (12 mg, 0.029 mmol), N,N-diisopropylethylamine (14 μL, 0.078 mmol). The resulting reaction mixture was stirred at r.t. for 2 min. 4-[(2-aminoethyl)amino]-2-(2,6-dioxo-3-piperidinyl)-1H-isoindole-1,3(2H)-dione⁵⁴ (22.1 mg, 0.032 mmol) was added to the mixture. Then, the mixture was stirred at r.t. for 1 h and purified by HPLC under acidic condition (5-95% CH₃CN in 0.1% aq. HCO₂H) to afford title compound. Yield: 12 mg (55%).

¹H NMR (500 MHz, CDCl₃) δ (ppm)=8.36 (1H, s), 7.53 (1H, t, J=7.8 Hz), 7.22 (1H, t, J=5.6 Hz), 7.14 (1H, d, J=6.8 Hz), 7.05 (1H, d, J=8.7 Hz), 6.49 (1H, t, J=5.7 Hz), 4.94 (1H, dd, J=5.2, 12.2 Hz), 4.07-4.01 (2H, m), 3.72-3.47 (28H, m), 3.44-3.29 (10H, m), 2.95-2.69 (3H, m), 2.18-2.10 (1H, m), 0.95 (3H, s).

¹³C NMR (126 MHz, CDCl₃) δ (ppm)=171.2, 169.4, 168.4, 167.7, 146.9, 136.4, 132.7, 116.9, 112.1, 110.6, 74.2, 74.1, 71.2, 71.1, 70.9, 70.8, 70.7, 70.6, 70.1, 50.9, 49.1, 42.3, 41.2, 38.6, 31.6, 22.9, 17.6.

MS (ESI) for C₃₆H₅₅N₁₀O₁₃ [M+H⁺] calculated 835.4, obtained 835.3.

### N,N′-(11-((2-((2-((2-(2,6-dioxopiperidin-3-yl)-1,3-dioxoisoindolin-4-yl)amino)ethyl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (SIM4)

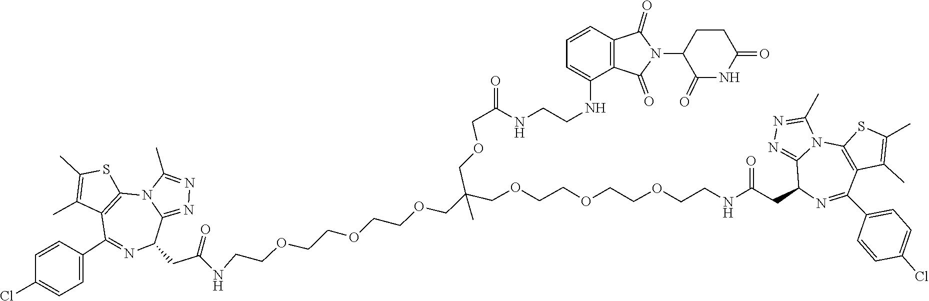 [SMILES: Cc1sc2c(c1C)C(c1ccc(Cl)cc1)=N[C@@H](CC(=O)NCCOCCOCCOCC(C)(COCCOCCOCCNC(=O)C[C@@H]1N=C(c3ccc(Cl)cc3)c3c(sc(C)c3C)-n3c(C)nnc31)COCC(=O)NCCNc1cccc3c1C(=O)N(C1CCC(=O)NC1=O)C3=O)c1nnc(C)n1-2]

To the 1-azido-11-((2-(2-(2-azidoethoxy)ethoxy)ethoxy)methyl)-N-(2-((2-(2,6-dioxopiperidin-3-yl)-1,3-dioxoisoindolin-4-yl)amino)ethyl)-11-methyl-3,6,9,13-tetraoxapentadecan-15-amide 23 (12.5 mg, 0.0158 mmol) in MeOH (0.8 mL), were added 10% wt palladium on carbon (2.5 mg). The resulting reaction mixture was stirred under hydrogen atmosphere at r.t. for 2 h. The mixture was then filtered on a celite pad and solvent evaporated. A pre stirred mixture of (+)-JQ1 carboxylic acid (12.5 mg, 0.031 mmol), COMU (13.4 mg, 0.031 mmol), N,N-diisopropylethylamine (13.6 μL, 0.078 mmol) in DMF (0.20 mL) was added to the concentrated crude. Then, the mixture was stirred at r.t. for 4 h and purified by HPLC under acidic condition (5-95% CH₃CN in 0.1% aq. HCO₂H) to afford title compound. Yield: 1.3 mg (5%).

¹H NMR (500 MHz, CD₃OD) δ (ppm)=8.53 (1H, s), 7.51 (1H, dd, J=7.3, 8.5 Hz), 7.44 (4H, d, J=8.4 Hz), 7.39 (4H, dd, J=2.2, 8.8 Hz), 7.08 (1H, d, J=8.6 Hz), 7.02 (1H, d, J=7.2 Hz), 4.99 (1H, ddd, J=2.0, 5.5, 12.6 Hz), 4.65-4.59 (2H, m), 3.92-3.86 (2H, m), 3.69-3.38 (32H, m), 3.31-3.25 (6H, m), 2.89-2.78 (1H, m), 2.75-2.62 (8H, m), 2.43 (6H, s), 2.12-2.03 (1H, m), 1.68 (6H, s), 0.87 (3H, s).

¹³C NMR (126 MHz, CD₃OD) δ (ppm)=174.7, 173.5, 172.9, 171.4, 170.3, 169.2, 166.1, 157.0, 152.2, 148.1, 138.1, 137.9, 137.3, 133.5, 133.2, 132.0, 131.4, 130.4, 129.8, 129.5, 118.0, 112.1, 111.3, 75.4, 74.7, 72.1, 71.6, 71.4, 70.7, 55.2, 42.6, 41.8, 40.6, 39.6, 38.7, 32.2, 30.8, 23.8, 18.0, 14.5.

HRMS (ESI) for C₇₂H₈₅Cl₂N₁₄O₁₄S₂ [M+2H⁺]/2 calculated 752.2633, obtained 752.2732

### N,N′-(11-((2-(2-((2-((2-(2,6-dioxopiperidin-3-yl)-1,3-dioxoisoindolin-4-yl)amino)ethyl)amino)-2-oxoethoxy)ethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (SIM5)

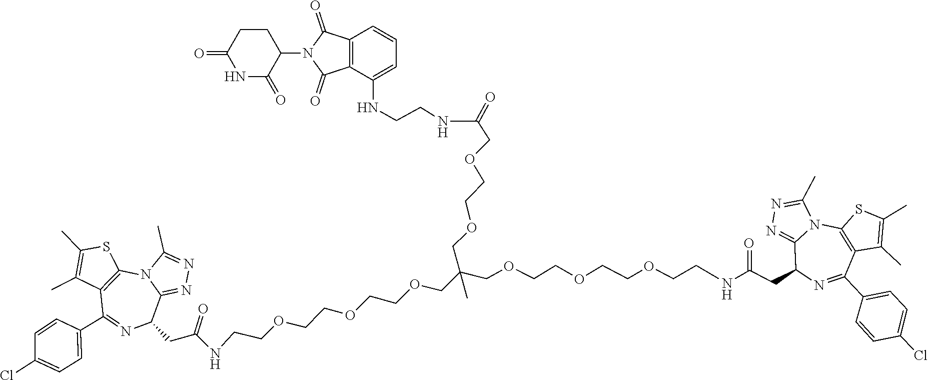 [SMILES: Cc1sc2c(c1C)C(c1ccc(Cl)cc1)=N[C@@H](CC(=O)NCCOCCOCCOCC(C)(COCCOCCOCCNC(=O)C[C@@H]1N=C(c3ccc(Cl)cc3)c3c(sc(C)c3C)-n3c(C)nnc31)COCCOCC(=O)NCCNc1cccc3c1C(=O)N(C1CCC(=O)NC1=O)C3=O)c1nnc(C)n1-2]

To a mixture of 1-azido-11-((2-(2-(2-azidoethoxy)ethoxy)ethoxy)methyl)-N-(2-((2-(2,6-dioxopiperidin-3-yl)-1,3-dioxoisoindolin-4-yl)amino)ethyl)-11-methyl-3,6,9,13,16-pentaoxaoctadecan-18-amide 24 (12 mg, 0.0144 mmol) in MeOH (0.80 mL), were added 10% wt palladium on carbon (2.5 mg). The resulting reaction mixture was stirred under hydrogen atmosphere at r.t. for 2 h. The mixture was then filtered on a celite pad and solvent evaporated. A pre stirred mixture of (+)-JQ1 carboxylic acid (13.8 mg, 0.035 mmol), COMU (14.8 mg, 0.035 mmol), N,N-diisopropylethylamine (15 μL, 0.086 mmol) in DMF (0.20 mL) was added to the concentrated crude. Then, the mixture was stirred at r.t. for 16 h and purified by HPLC under acidic condition (5-95% CH₃CN in 0.1% aq. HCO₂H) to afford title compound. Yield: 1.8 mg (8%).

¹H NMR (500 MHz, CD₃OD) δ (ppm)=7.52 (1H, dd, J=7.4, 8.2 Hz), 7.44 (4H, d, J=8.4 Hz), 7.39 (4H, dd, J=1.4, 8.7 Hz), 7.10 (1H, d, J=8.6 Hz), 7.02 (1H, d, J=7.1 Hz), 5.00 (1H, dd, J=5.3, 13.0 Hz), 4.62 (2H, dd, J=5.2, 8.9 Hz), 3.99-3.94 (2H, m), 3.67-3.38 (36H, m), 3.36-3.23 (7H, m), 2.88-2.78 (1H, m), 2.76-2.61 (2H, m), 2.68 (6H, s), 2.43 (6H, s), 2.12-2.04 (1H, m), 1.69 (6H, s), 0.88 (3H, s).

¹³C NMR (126 MHz, CD₃OD) δ (ppm)=174.6, 173.5, 172.9, 171.4, 170.5, 169.2, 166.1, 157.0, 152.2, 148.0, 138.1, 137.9, 137.3, 133.5, 133.2, 132.0, 131.4, 129.8, 118.1, 112.1, 111.4, 74.8, 72.1, 71.9, 71.7, 71.6, 71.4, 71.3, 70.7, 55.2, 42.6, 42.0, 40.6, 39.4, 38.7, 32.2, 30.8, 23.7, 18.0, 14.5, 12.9, 11.6.

HRMS (ESI) for C₇₄H₈₉Cl₂N₁₄O₁₅S₂ [M+H⁺] calculated 1547.5445, obtained 1547.5989.

### Synthesis of Compounds SIM6, Cis-SIM1

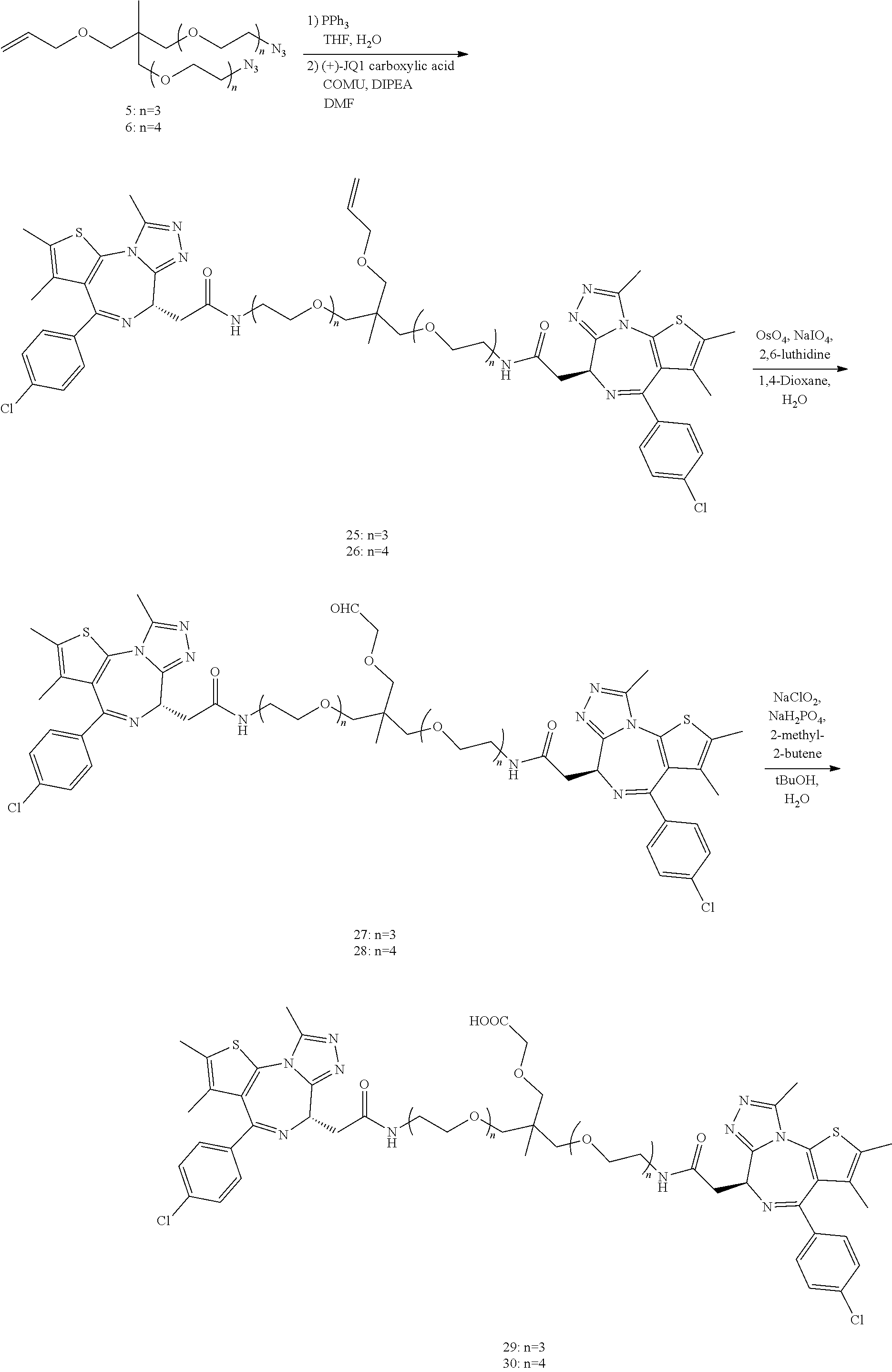

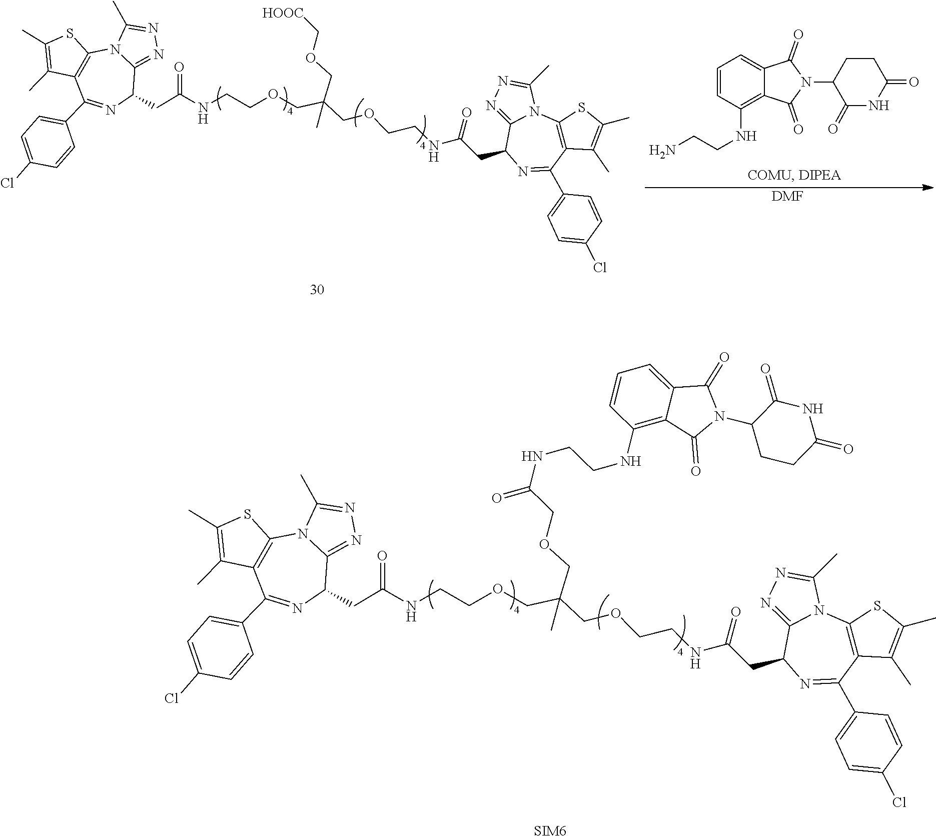

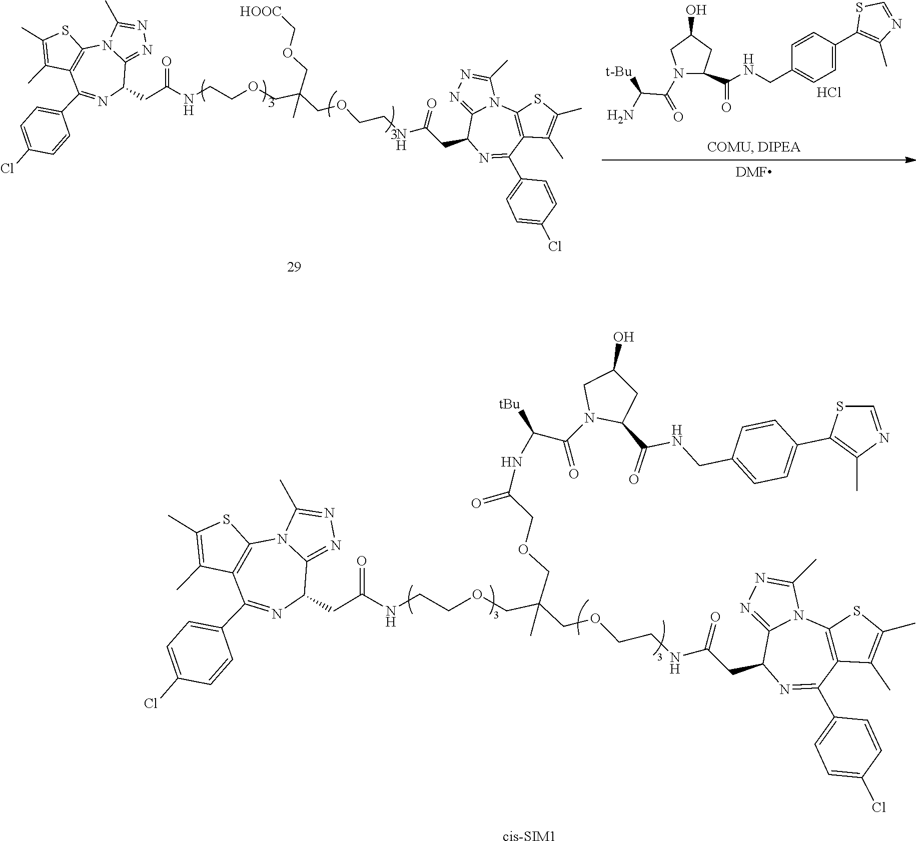

### N,N′-(11-((allyloxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (25)

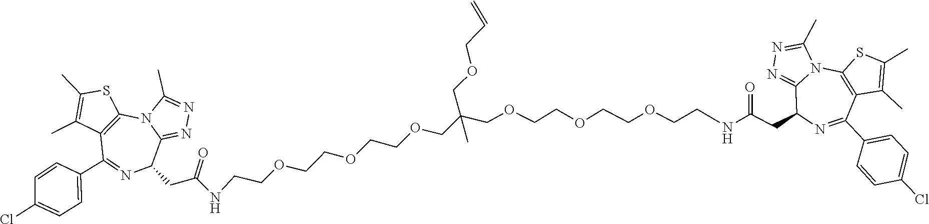 [SMILES: C=CCOCC(C)(COCCOCCOCCNC(=O)C[C@@H]1N=C(c2ccc(Cl)cc2)c2c(sc(C)c2C)-n2c(C)nnc21)COCCOCCOCCNC(=O)C[C@@H]1N=C(c2ccc(Cl)cc2)c2c(sc(C)c2C)-n2c(C)nnc21]

To a mixture of 11-((allyloxy)methyl)-1,21-diazido-11-methyl-3,6,9,13,16,19-hexaoxahenicosane 5 (25 mg, 0.053 mmol) in THE (0.53 mL), was added PPh₃ (41.7 mg, 0.16 mmol). The resulting mixture was stirred at 50° C. for 1 h. H₂O (0.05 mL) was added to the reaction mixture. Then, the mixture was stirred at 50° C. for 1 h and concentrated. A pre stirred mixture of (+)-JQ1 carboxylic acid (64 mg, 0.16 mmol), COMU (20.5 mg, 0.16 mmol), N,N-diisopropylethylamine (55.4 μL, 0.32 mmol) in DMF (0.42 mL) was added to the concentrated crude. Then, the resulting mixture was stirred at r.t. for 1 h and purified by HPLC under acidic condition (5-95% CH₃CN in 0.1% aq. HCO₂H) to afford title compound. Yield: 17 mg (27%).

¹H NMR (400 MHz, CDCl₃) δ (ppm)=7.40 (4H, d, J=8.1 Hz), 7.31 (4H, d, J=7.9 Hz), 6.98-6.89 (2H, m), 5.93-5.80 (1H, m), 5.23 (1H, d, J=17.2 Hz), 5.12 (1H, d, J=11.0 Hz), 4.65 (2H, t, J=6.8 Hz), 3.93 (2H, d, J=5.2 Hz), 3.72-3.24 (32H, m), 2.66 (6H, s), 2.39 (6H, s), 1.66 (6H, s), 0.94 (3H, s).

¹³C NMR (101 MHz, CDCl₃) δ (ppm)=170.7, 164.0, 155.8, 150.0, 136.9, 136.8, 135.4, 132.3, 131.0, 130.9, 130.6, 130.0, 116.3, 74.1, 73.1, 72.4, 71.2, 70.7, 70.6, 70.5, 70.0, 54.5, 41.1, 39.6, 39.2, 17.6, 14.5, 13.2, 11.9.

MS (ESI) for C₅₈H₇₃Cl₂N₁₀O₉S₂ [M+H⁺] calculated 1187.4, obtained 1187.4.

### N,N′-(14-((allyloxy)methyl)-14-methyl-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (26)

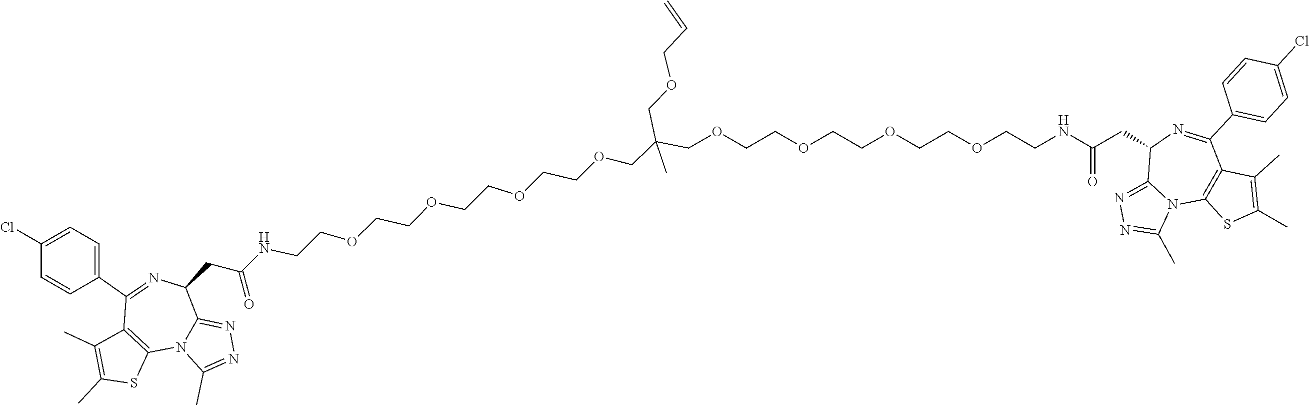 [SMILES: C=CCOCC(C)(COCCOCCOCCOCCNC(=O)C[C@@H]1N=C(c2ccc(Cl)cc2)c2c(sc(C)c2C)-n2c(C)nnc21)COCCOCCOCCOCCNC(=O)C[C@@H]1N=C(c2ccc(Cl)cc2)c2c(sc(C)c2C)-n2c(C)nnc21]

To a mixture of 14-((allyloxy)methyl)-1,27-diazido-14-methyl-3,6,9,12,16,19,22,25-octaoxaheptacosane 6 (67 mg, 0.119 mmol) in THE (1.2 mL), was added PPh₃ (93.5 mg, 0.36 mmol). The resulting mixture was stirred at 50° C. for 1 h. H₂O (0.12 mL) was added to the reaction mixture. Then, the mixture was stirred at 50° C. for 1 h and concentrated. A pre stirred mixture of (+)-JQ1 carboxylic acid (143 mg, 0.36 mmol), COMU (153 mg, 0.36 mmol), N,N-diisopropylethylamine (124 μL, 0.72 mmol) in DMF (0.95 mL) was added to the concentrated crude. Then, the resulting mixture was stirred at r.t. for 1 h and purified by HPLC under acidic condition (5-95% CH₃CN in 0.1% aq. HCO₂H) to afford title compound. Yield: 87 mg (57%).

¹H NMR (500 MHz, CDCl₃) δ (ppm)=7.38 (4H, d, J=8.2 Hz), 7.28 (4H, d, J=8.0 Hz), 7.09-6.93 (2H, m), 5.89-5.76 (1H, m), 5.20 (1H, d, J=18.0 Hz), 5.08 (1H, d, J=10.2 Hz), 4.62 (2H, t, J=6.0 Hz), 3.89 (2H, d, J=4.8 Hz), 3.82-3.18 (40H, m), 2.62 (6H, s), 2.36 (6H, s), 1.64 (6H, s), 0.90 (3H, s).

¹³C NMR (126 MHz, CDCl₃) δ (ppm)=170.5, 163.7, 155.6, 149.7, 136.6, 135.2, 132.0, 130.8, 130.4, 129.8, 128.6, 116.0, 73.9, 72.9, 72.1, 71.0, 70.6, 70.5, 70.3, 69.7, 54.3, 40.9, 39.4, 38.9, 17.4, 14.3, 13.0, 11.7.

MS (ESI) for C₆₂H₈₁Cl₂N₁₀O₁₁S₂ [M+H⁺] calculated 1275.5, obtained 1275.5.

### N,N′-(11-methyl-11-((2-oxoethoxy)methyl)-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (27)

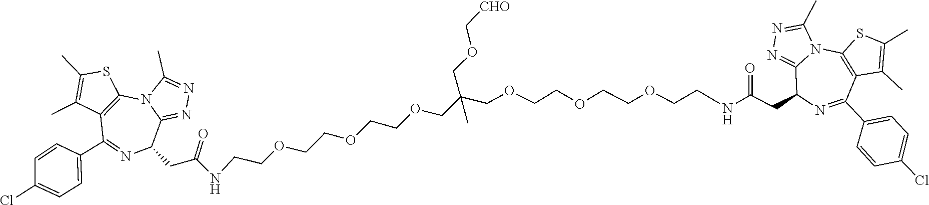 [SMILES: Cc1sc2c(c1C)C(c1ccc(Cl)cc1)=N[C@@H](CC(=O)NCCOCCOCCOCC(C)(COCC=O)COCCOCCOCCNC(=O)C[C@@H]1N=C(c3ccc(Cl)cc3)c3c(sc(C)c3C)-n3c(C)nnc31)c1nnc(C)n1-2]

To a mixture of N,N′-(11-((allyloxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) 25 (17 mg, 0.014 mmol) in H₂O (0.09 mL) and 1,4-dioxane (0.26 mL), were added 2,6-luthidine (3.3 μL, 0.029 mmol), osmium tetroxide 4% in H₂O (1.8 μL, 0.0003 mmol), sodium periodate (12.2 mg, 0.058 mmol). The resulting reaction mixture was stirred at r.t. for 8 h. The reaction mixture was quenched with Na₂SO₃ (aq) and extracted with dichloromethane. The organic layer was concentrated and the remaining residue was purified by flush column chromatography to afford title compound. Yield: 14 mg (82%).

¹H NMR (400 MHz, CDCl₃) δ (ppm)=9.69 (1H, s), 7.40 (4H, d, J=7.9 Hz), 7.31 (4H, d, J=8.0 Hz), 7.00-6.90 (2H, m), 4.66 (2H, t, J=6.7 Hz), 4.04-3.98 (2H, m), 3.74-3.27 (32H, m), 2.65 (6H, s), 2.39 (6H, s), 1.66 (6H, s), 0.97 (3H, s).

¹³C NMR (101 MHz, CDCl₃) δ (ppm)=202.4, 170.7, 163.9, 155.8, 150.0, 136.8, 132.3, 131.0, 130.8, 130.6, 130.0, 128.8, 74.7, 73.9, 71.2, 70.7, 70.6, 70.0, 54.5, 41.2, 39.5, 39.2, 29.8, 17.5, 14.5, 13.2, 12.0.

MS (ESI) for C₅₇H₇₁Cl₂N₁₀O₁₀S₂[M+H⁺] calculated 1189.4, obtained 1189.4.

### N,N′-(14-methyl-14-((2-oxoethoxy)methyl)-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (28)

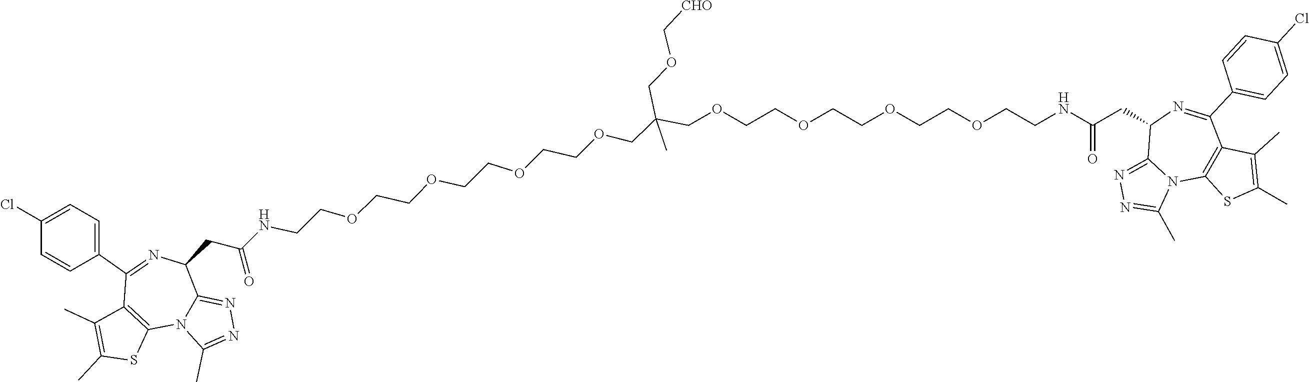 [SMILES: Cc1sc2c(c1C)C(c1ccc(Cl)cc1)=N[C@@H](CC(=O)NCCOCCOCCOCCOCC(C)(COCC=O)COCCOCCOCCOCCNC(=O)C[C@@H]1N=C(c3ccc(Cl)cc3)c3c(sc(C)c3C)-n3c(C)nnc31)c1nnc(C)n1-2]

To a mixture of N,N′-(14-((allyloxy)methyl)-14-methyl-3,6,9,12,16,19,22,25-octaoxaheptacosane-1,27-diyl)bis(2-((S)-4-(4-chlorophenyl)-2,3,9-trimethyl-6H-thieno[3,2-][1,2,4]triazolo[4,3-a][1,4]diazepin-6-yl)acetamide) 26 (87 mg, 0.068 mmol) in H₂O (0.41 mL) and 1,4-dioxane (1.2 mL), were added 2,6-luthidine (15.9 μL, 0.136 mmol), osmium tetroxide 4% in H₂O (1.8 μL, 0.0014 mmol), sodium periodate (58 mg, 0.27 mmol). The resulting reaction mixture was stirred at r.t. for 8 h. The reaction mixture was quenched with Na₂SO₃ (aq) and extracted with dichloromethane. The organic layer was concentrated and the remaining residue was purified by flush column chromatography to afford title compound. Yield: 74 mg (85%).

¹H NMR (500 MHz, CDCl₃) δ (ppm)=9.70 (1H, s), 7.41 (4H, d, J=8.3 Hz), 7.32 (4H, d, J=8.6 Hz), 6.93-6.85 (2H, m), 4.65 (2H, t, J=6.9 Hz), 4.07-4.01 (2H, m), 3.73-3.45 (36H, m), 3.44-3.29 (6H, m), 2.66 (6H, s), 2.39 (6H, s), 1.67 (6H, s), 0.94 (3H, s).

¹³C NMR (126 MHz, CDCl₃) δ (ppm)=202.0, 170.6, 163.7, 155.7, 149.8, 136.7, 136.7, 132.2, 130.9, 130.7, 130.4, 129.9, 128.6, 76.9, 74.6, 73.8, 71.0, 70.6, 70.5, 70.4, 70.3, 69.8, 54.4, 41.0, 39.4, 39.0, 17.4, 14.3, 13.0, 11.8.

MS (ESI) for C₆₁H₇₉Cl₂N₁₀O₁₂S₂ [M+H⁺] calculated 1277.5, obtained 1277.5.

### 1-((S)-4-(4-chlorophenyl)-2,3,9-trimethyl-6H-thieno[3,2-f][1,2,4]triazolo[4,3-a] [1,4]diazepin-6-yl)-14-(13-((S)-4-(4-chlorophenyl)-2,3,9-trimethyl-6H-thieno[3,2-f][1,2,4]triazolo[4,3-a][1,4]diazepin-6-yl)-12-oxo-2,5,8-trioxa-11-azatridecyl)-14-methyl-2-oxo-6,9,12,16-tetraoxa-3-azaoctadecan-18-oic acid (29)

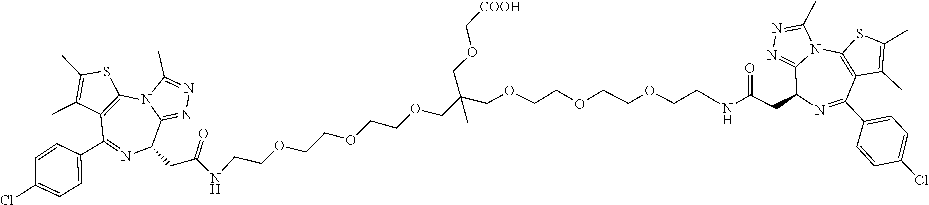 [SMILES: Cc1sc2c(c1C)C(c1ccc(Cl)cc1)=N[C@@H](CC(=O)NCCOCCOCCOCC(C)(COCCOCCOCCNC(=O)C[C@@H]1N=C(c3ccc(Cl)cc3)c3c(sc(C)c3C)-n3c(C)nnc31)COCC(=O)O)c1nnc(C)n1-2]

To a mixture of N,N′-(11-methyl-11-((2-oxoethoxy)methyl)-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) 27 (14 mg, 0.012 mmol) in t-BuOH (0.21 mL), were added 2M 2-methyl-2-butene in THF (59 μL, 0.12 mmol), NaH₂PO₄ (1.4 mg, 0.012 mmol), sodium chlorite (4.6 mg, 0.047 mmol) in H₂O (0.07 mL). The resulting reaction mixture was stirred at r.t. for 4 h. The reaction mixture was evaporated and purified by HPLC under acidic condition (5-95% CH₃CN in 0.1% aq. HCO₂H) to afford title compound. Yield: 6.2 mg (44%).

¹H NMR (500 MHz, CDCl₃) δ (ppm)=7.41 (4H, d, J=8.7 Hz), 7.32 (4H, d, J=8.7 Hz), 4.68 (2H, t, J=7.1 Hz), 4.11-4.05 (2H, m), 3.72-3.31 (34H, m), 2.66 (6H, s), 2.40 (6H, s), 1.68 (6H, s), 0.94 (3H, s).

¹³C NMR (126 MHz, CDCl₃) δ (ppm)=170.7, 164.0, 155.8, 150.0, 137.0, 136.7, 132.1, 131.1, 130.9, 130.0, 128.9, 74.4, 71.4, 70.8, 70.6, 70.1, 54.6, 41.0, 39.6, 39.1, 17.8, 14.5, 13.2, 11.9.

MS (ESI) for C₅₇H₇₁C₁₂N₁₀O₁₁S₂ [M+H⁺] calculated 1205.4, found 1205.4.

### 1-((S)-4-(4-chlorophenyl)-2,3,9-trimethyl-6H-thieno[3,2-f][1,2,4]triazolo[4,3-a][1,4]diazepin-6-yl)-17-(16-((S)-4-(4-chlorophenyl)-2,3,9-trimethyl-6H-thieno[3,2-f][1,2,4]triazolo[4,3-a][1,4]diazepin-6-yl)-15-oxo-2,5,8,11-tetraoxa-14-azahexadecyl)-17-methyl-2-oxo-6,9,12,15,19-pentaoxa-3-azahenicosan-21-oic acid (30)

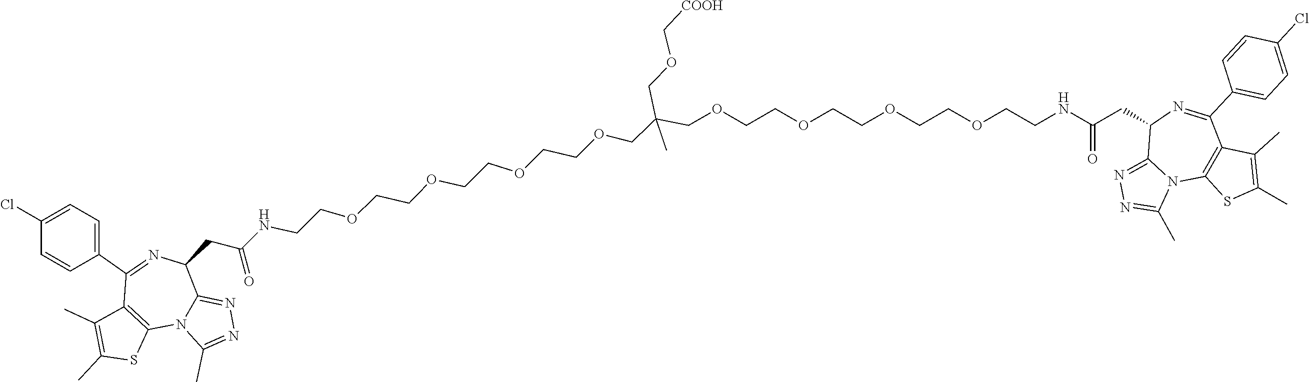 [SMILES: Cc1sc2c(c1C)C(c1ccc(Cl)cc1)=N[C@@H](CC(=O)NCCOCCOCCOCCOCC(C)(COCCOCCOCCOCCNC(=O)C[C@@H]1N=C(c3ccc(Cl)cc3)c3c(sc(C)c3C)-n3c(C)nnc31)COCC(=O)O)c1nnc(C)n1-2]

To a mixture of N,N′-(14-methyl-14-((2-oxoethoxy)methyl)-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide) 28 (74 mg, 0.058 mmol) in t-butanol (1.0 mL), were added 2M 2-methyl-2-butene in THF (290 μL, 0.58 mmol), NaH₂PO₄ (7.0 mg, 0.058 mmol), sodium chlorite (22.4 mg, 0.23 mmol) in H₂O (0.35 mL). The resulting reaction mixture was stirred at r.t. for 4 h. The reaction mixture was evaporated and purified by HPLC under acidic condition (5-95% CH₃CN in 0.1% aq. HCO₂H) to afford title compound. Yield: 63 mg (84%).

¹H NMR (500 MHz, CDCl₃) δ (ppm)=7.41 (4H, d, J=8.8 Hz), 7.33 (4H, d, J=8.6 Hz), 7.04-6.94 (2H, m), 4.66 (2H, t, J=7.1 Hz), 4.09-4.05 (2H, m), 3.74-3.43 (36H, m), 3.41-3.30 (6H, m), 2.67 (6H, s), 2.40 (6H, s), 1.68 (7H, s), 0.94 (3H, s).

¹³C NMR (126 MHz, CDCl₃) δ (ppm)=172.7, 170.6, 163.9, 155.6, 149.9, 136.8, 136.6, 132.0, 131.0, 130.7, 129.9, 128.7, 74.7, 74.2, 71.1, 70.7, 70.5, 70.3, 69.7, 69.1, 54.4, 40.9, 39.5, 38.9, 17.5, 14.4, 13.1, 11.7.

MS (ESI) for C₆₁H₇₉C₁₂N₁₀O₁₂S₂ [M+H⁺] calculated 1293.5, obtained 1293.4.

### N,N′-(14-((2-((2-((2-(2,6-dioxopiperidin-3-yl)-1,3-dioxoisoindolin-4-yl)amino)ethyl)amino)-2-oxoethoxy)methyl)-14-methyl-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (SIM6)

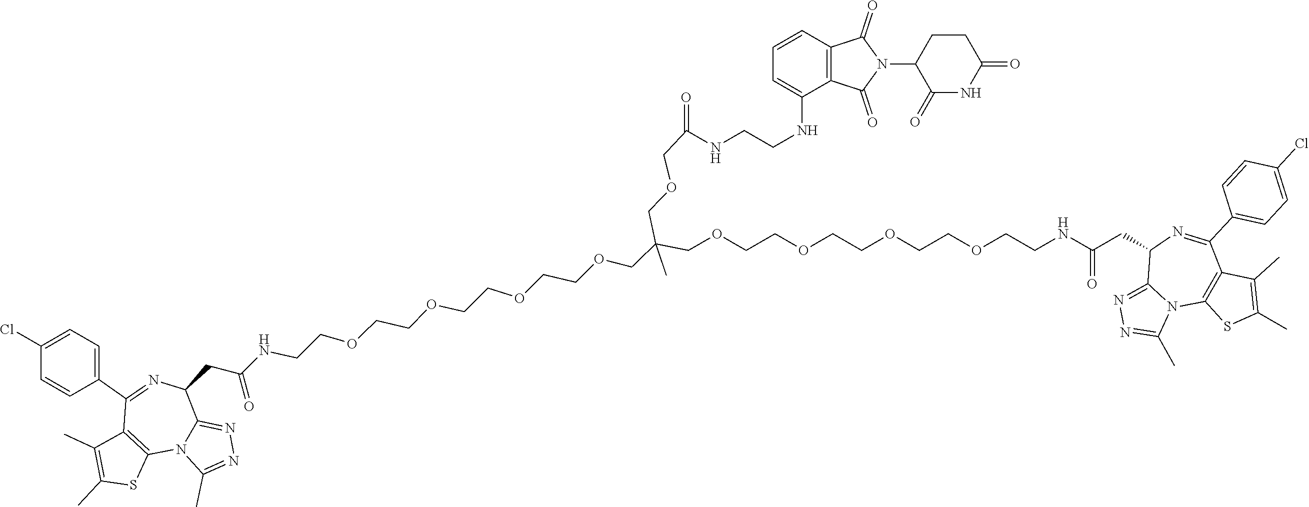 [SMILES: Cc1sc2c(c1C)C(c1ccc(Cl)cc1)=N[C@@H](CC(=O)NCCOCCOCCOCCOCC(C)(COCCOCCOCCOCCNC(=O)C[C@@H]1N=C(c3ccc(Cl)cc3)c3c(sc(C)c3C)-n3c(C)nnc31)COCC(=O)NCCNc1cccc3c1C(=O)N(C1CCC(=O)NC1=O)C3=O)c1nnc(C)n1-2]

To a mixture of 1-((S)-4-(4-chlorophenyl)-2,3,9-trimethyl-6H-thieno[3,2-f][1,2,4]triazolo[4,3-a][1,4]diazepin-6-yl)-17-(16-((S)-4-(4-chlorophenyl)-2,3,9-trimethyl-6H-thieno[3,2-f][1,2,4]triazolo[4,3-a][1,4]diazepin-6-yl)-15-oxo-2,5,8,11-tetraoxa-14-azahexadecyl)-17-methyl-2-oxo-6,9,12,15,19-pentaoxa-3-azahenicosan-21-oic acid 30 (10 mg, 0.0078 mmol) in DMF (0.12 mL), were added COMU (3.7 mg, 0.0086 mmol), N,N-diisopropylethylamine (4.1 μL, 0.023 mmol). The resulting reaction mixture was stirred at r.t. for 5 min. The mixture was added to 4-[(2-aminoethyl)amino]-2-(2,6-dioxo-3-piperidinyl)-1H-isoindole-1,3(2H)-dione⁵⁴ (3.7 mg, 0.012 mmol). Then, the mixture was stirred at r.t. for 1 h and purified by HPLC under acidic condition (5-95% CH₃CN in 0.1% aq. HCO₂H) to afford title compound. Yield: 5.0 mg (41%).

¹H NMR (500 MHz, CD₃OD) δ (ppm)=7.51 (1H, dd, J=7.3, 8.5 Hz), 7.44 (4H, d, J=8.6 Hz), 7.39 (4H, dd, J=1.3, 8.5 Hz), 7.09 (1H, d, J=8.6 Hz), 7.01 (1H, d, J=6.9 Hz), 5.00 (1H, ddd, J=2.3, 5.3, 12.8 Hz), 4.65-4.59 (2H, m), 3.91-3.86 (2H, m), 3.66-3.38 (40H, m), 3.30-3.23 (6H, m), 2.89-2.78 (1H, m), 2.76-2.61 (2H, m), 2.69 (6H, s), 2.43 (6H, s), 2.11-2.04 (1H, m), 1.69 (6H, s), 0.85 (3H, s).

¹³C NMR (126 MHz, CD₃OD) δ (ppm)=174.6, 173.5, 172.9, 171.3, 170.6, 169.2, 166.1, 157.1, 152.1, 148.2, 138.2, 137.9, 137.3, 133.9, 133.5, 133.2, 132.0, 131.4, 129.8, 118.1, 112.2, 111.4, 75.6, 74.8, 72.1, 71.7, 71.6, 71.4, 70.7, 55.2, 50.2, 42.6, 41.9, 40.6, 39.6, 38.8, 32.2, 23.8, 18.0, 14.4, 12.9, 11.6. HRMS (ESI) for C₇₆H₉₃Cl₂N₁₄O₁₆S₂ [M+H⁺] calculated 1591.5707, obtained 1591.5343

### N,N′-(11-((2-(((S)-1-((2S,4S)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (cis-SIM1)

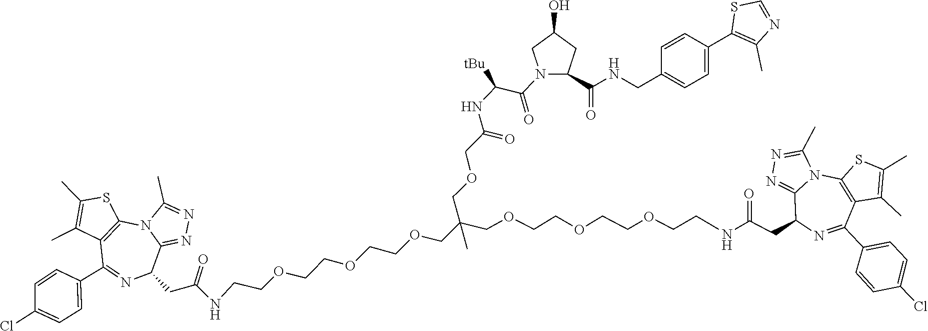 [SMILES: Cc1ncsc1-c1ccc(CNC(=O)[C@@H]2C[C@H](O)CN2C(=O)[C@@H](NC(=O)COCC(C)(COCCOCCOCCNC(=O)C[C@@H]2N=C(c3ccc(Cl)cc3)c3c(sc(C)c3C)-n3c(C)nnc32)COCCOCCOCCNC(=O)C[C@@H]2N=C(c3ccc(Cl)cc3)c3c(sc(C)c3C)-n3c(C)nnc32)C(C)(C)C)cc1]

To a mixture of 1-((S)-4-(4-chlorophenyl)-2,3,9-trimethyl-6H-thieno[3,2-f][1,2,4]triazolo[4,3-a][1,4]diazepin-6-yl)-14-(13-((S)-4-(4-chlorophenyl)-2,3,9-trimethyl-6H-thieno[3,2-f][1,2,4]triazolo[4,3-a][1,4]diazepin-6-yl)-12-oxo-2,5,8-trioxa-11-azatridecyl)-14-methyl-2-oxo-6,9,12,16-tetraoxa-3-azaoctadecan-18-oic acid 29 (5.2 mg, 0.0043 mmol) in DMF (0.07 mL), were added COMU (2.0 mg, 0.0047 mmol), N,N-diisopropylethylamine (2.3 μL, 0.013 mmol). The resulting reaction mixture was stirred at r.t. for 5 min. The mixture was added to cis-VH032 amine hydrochloride 9 (3.0 mg, 0.0065 mmol). Then, the mixture was stirred at r.t. for 1 h and purified by HPLC under acidic condition (5-95% CH₃CN in 0.1% aq. HCO₂H) to afford title compound. Yield: 4.4 mg (63%).

¹H NMR (500 MHz, CD₃OD) δ (ppm)=8.85 (1H, s), 7.48-7.36 (12H, m), 4.65-4.59 (3H, m), 4.58-4.50 (2H, m), 4.40-4.32 (2H, m), 4.01-3.89 (3H, m), 3.73-3.34 (35H, m), 3.27 (1H, d, J=5.1 Hz), 2.68 (6H, s), 2.46 (3H, s), 2.44-2.36 (1H, m), 2.43 (6H, s), 1.96 (1H, dt, J=4.4, 13.3 Hz), 1.69 (6H, s), 1.02 (9H, s), 0.97 (3H, s).

¹³C NMR (126 MHz, CD₃OD) δ (ppm)=174.8, 172.9, 172.0, 171.8, 166.1, 157.1, 152.8, 152.1, 149.1, 140.0, 138.2, 137.9, 133.5, 133.2, 132.0, 131.4, 130.4, 129.8, 129.1, 75.4, 74.8, 74.7, 72.2, 71.7, 71.5, 71.4, 70.7, 61.0, 57.9, 57.6, 55.2, 43.9, 42.1, 40.6, 38.8, 37.8, 36.8, 27.0, 18.0, 15.9.

MS (ESI) for C₇₉H₉₉Cl₂N₁₄O₁₃S₃[M+H⁺] calculated 1617.6050, obtained 1617.5716.

### Synthesis of (R,S)-SIM1

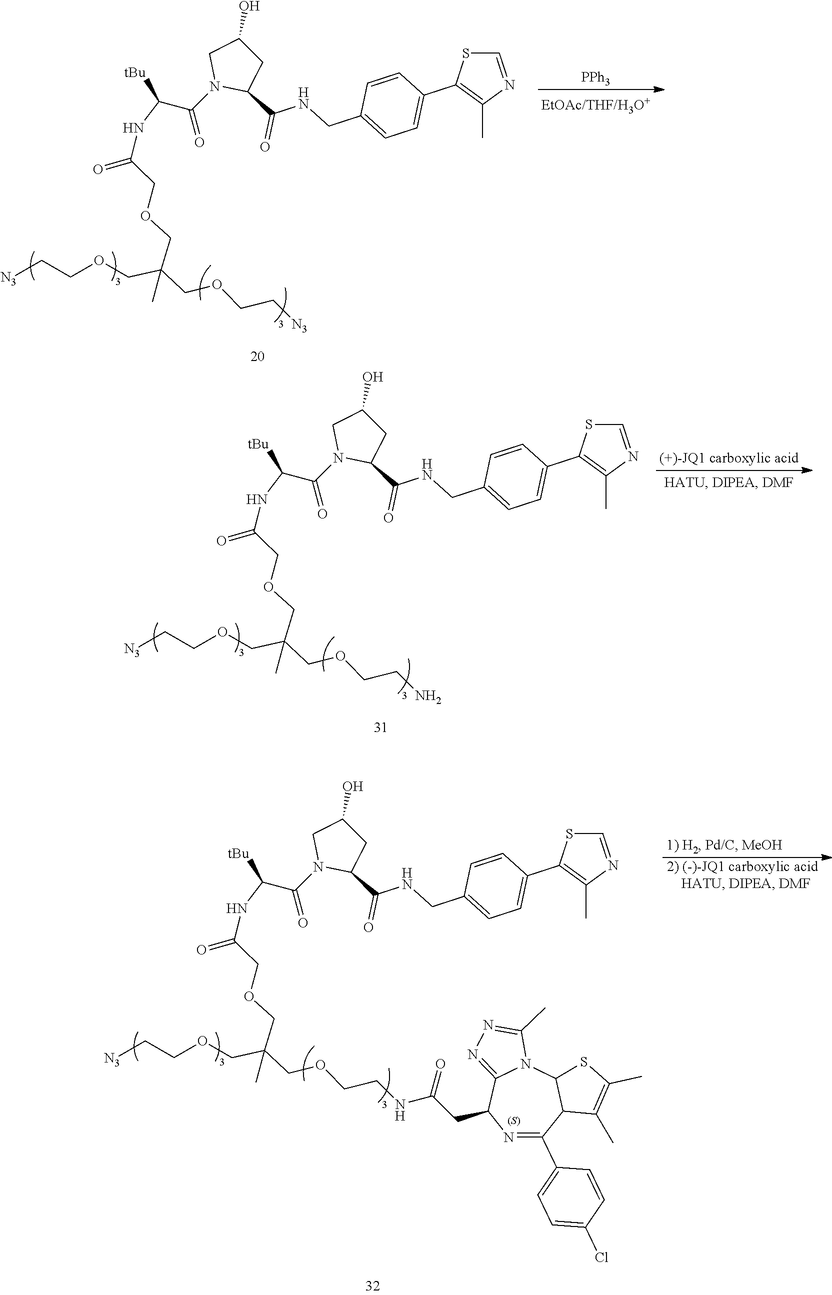

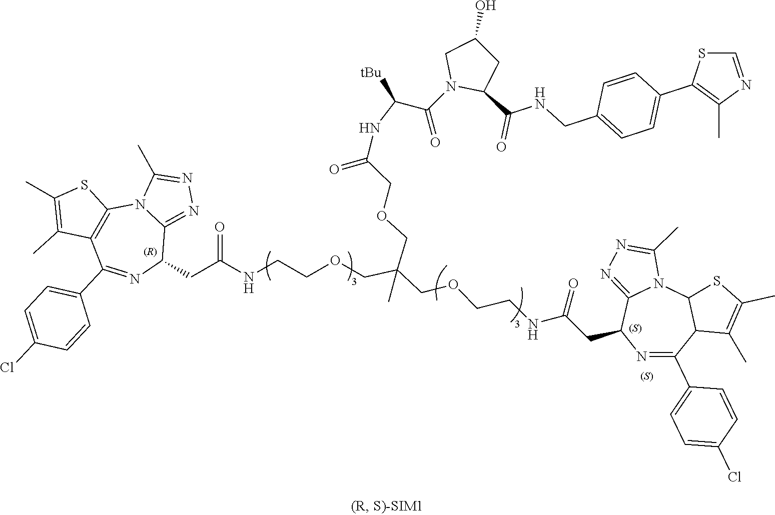

### (2S,4R)-1-((17S)-1-amino-11-((2-(2-(2-azidoethoxy)ethoxy)ethoxy)methyl)-17-(tert-butyl)-11-methyl-15-oxo-3,6,9,13-tetraoxa-16-azaoctadecan-18-oyl)-4-hydroxy-N-(4-(4-methylthiazol-5-yl)benzyl)pyrrolidine-2-carboxamide (31)

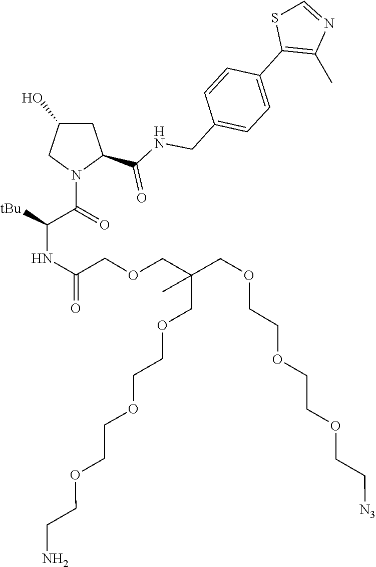 [SMILES: Cc1ncsc1-c1ccc(CNC(=O)[C@@H]2C[C@@H](O)CN2C(=O)[C@@H](NC(=O)COCC(C)(COCCOCCOCCN)COCCOCCOCCN=[N+]=[N-])C(C)(C)C)cc1]

Triphenyl phosphine (15 mg, 0.057 mmol) in ethyl acetate (1.5 mL) was added dropwise over a 3 h period to compound 20 (53 mg, 0.058 mmol) in EtOAc/THF/HCl 1M (4 mL, 4:1:5) at r.t. The reaction mixture was vigorously stirred overnight before 4M HCl (2 mL) was added and the ethyl acetate layer was removed. The aqueous layer was washed with EtOAc and concentrated. The crude was purified by HPLC (5-95% CH₃CN in water with 0.1% ammonia) to give 11 mg of the starting material 20 and 10 mg (24%, based on the recovered starting material) of a desired monoamine 31.

¹H NMR (500 MHz, CDCl₃) δ (ppm)=8.67 (1H, s), 8.52 (1H, br), 7.38-7.33 (4H, m), 7.17 (1H, m), 4.67 (1H, m), 4.55-4.47 (3H, m), 4.38 (1H, m), 4.07-3.94 (4H, m), 3.70-3.21 (31H, m), 2.98 (2H, m), 2.51 (3H, s), 2.31 (1H, s), 2.23 (1H, s), 1.02-0.93 (12H, m).

¹³C NMR (126 MHz, CDCl₃) δ (ppm)=171.4, 171.03, 170.98, 170.72, 170.65, 169.4, 150.2, 148.4, 138.51, 138.47, 131.6, 130.67, 130.66, 129.36, 129.35, 127.97, 74.2, 74.1, 74.0, 73.9, 73.1, 71.1, 70.87, 70.84, 70.80, 71.72, 70.70, 70.62, 70.45, 70.42, 70.38, 70.34, 70.3, 70.2, 70.2, 70.0, 69.9, 68.6, 68.5, 59.0, 57.3, 57.0, 50.6, 43.0, 42.99, 41.03, 41.01, 39.8, 37.07, 36.98, 35.17, 35.10, 26.4, 17.5, 16.0

MS (ESI) for C₄₁H₆₇N₈O₁₁S₁ [M+H⁺]⁺ calculated 879.5, obtained 879.5.

### (2S,4R)-1-((20S)-14-((2-(2-(2-azidoethoxy)ethoxy)ethoxy)methyl)-20-(tert-butyl)-1-((S)-4-(4-chlorophenyl)-2,3,9-trimethyl-6H-thieno[3,2-f][1,2,4]triazolo[4,3-a][1,4]diazepin-6-yl)-14-methyl-2,18-dioxo-6,9,12,16-tetraoxa-3,19-diazahenicosan-21-oyl)-4-hydroxy-N-(4-(4-methylthiazol-5-yl)benzyl)pyrrolidine-2-carboxamide (32)

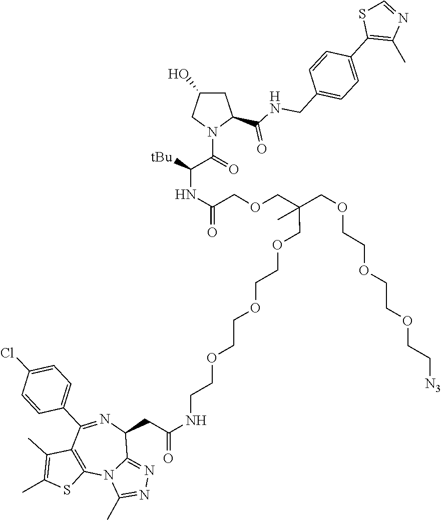 [SMILES: Cc1ncsc1-c1ccc(CNC(=O)[C@@H]2C[C@@H](O)CN2C(=O)[C@@H](NC(=O)COCC(C)(COCCOCCOCCN=[N+]=[N-])COCCOCCOCCNC(=O)C[C@@H]2N=C(c3ccc(Cl)cc3)c3c(sc(C)c3C)-n3c(C)nnc32)C(C)(C)C)cc1]

A pre stirred mixture of (+)-JQ1 carboxylic acid (4.5 mg, 0.011 mmol), HATU (4.2 mg, 0.011 mmol), N,N-diisopropylethylamine (5 μL, 0.03 mmol) in DMF (0.2 mL) was added to compound 31 (10 mg, 0.011 mmol). The resulting mixture was stirred at r.t. for 1 h and purified by HPLC (5-95% CH₃CN in 0.1% aq. HCO₂H) to afford the title compound 32. Yield: 12 mg (86%).

¹H NMR (500 MHz, CDCl₃) δ (ppm)=8.67 (1H, s), 7.69 (1H, m), 7.43-7.28 (10H, m), 7.15 (1H, d, J=9.0 Hz), 4.82 (1H, m), 4.64 (2H, m), 4.54 (1H, m), 4.47 (1H, m), 4.35 (1H, dd, J=5.4, 16.0 Hz), 4.10 (1H, m), 4.04 (1H, m), 3.95 (1H, m), 3.70-3.18 (38H, m), 2.62 (3H, s), 2.51 (3H, s), 2.48 (1H, m), 2.39 (3H, s), 2.14 (1H, m), 1.65 (3H, s), 0.97 (9H, s), 0.95 (1.5H, s), 0.93 (1.5H, s). ¹³C NMR (126 MHz, CDCl₃) δ (ppm)=171.2, 171.1, 170.8, 170.55, 170.52, 163.9, 162.3, 155.8, 150.2, 149.8, 148.4, 138.3, 136.7, 132.0, 131.7, 131.0, 130.8, 130.7, 129.9, 129.5, 128.7, 128.1, 74.5, 73.9, 73.8, 73.5, 73.0, 72.7, 71.1, 70.95, 70.92, 70.8, 70.7, 70.51, 70.47, 70.3, 70.24, 70.21, 70.0, 58.7, 57.0, 56.6, 54.3, 50.7, 43.2, 40.9, 39.5, 38.4, 38.3, 36.2, 35.3, 35.2, 26.4, 17.54, 17.48, 16.0, 14.4, 13.1, 11.8.

MS (ESI) for C₈₀H₈₃ClN₁₂O₁₂S₂[M+2H⁺]²⁺ calculated 631.3, obtained 631.8.

### (2S,4R)-1-((20S)-20-(tert-butyl)-1-((R)-4-(4-chlorophenyl)-2,3,9-trimethyl-6H-thieno[3,2-f][1,2,4]triazolo[4,3-a][1,4]diazepin-6-yl)-14-(13-((S)-4-(4-chlorophenyl)-2,3,9-trimethyl-6H-thieno[3,2-f][1,2,4]triazolo[4,3-a][1,4]diazepin-6-yl)-12-oxo-2,5,8-trioxa-11-azatridecyl)-14-methyl-2,18-dioxo-6,9,12,16-tetraoxa-3,19-diazahenicosan-21-oyl)-4-hydroxy-N-(4-(4-methylthiazol-5-yl)benzyl)pyrrolidine-2-carboxamide ((R,S)-SIM1)

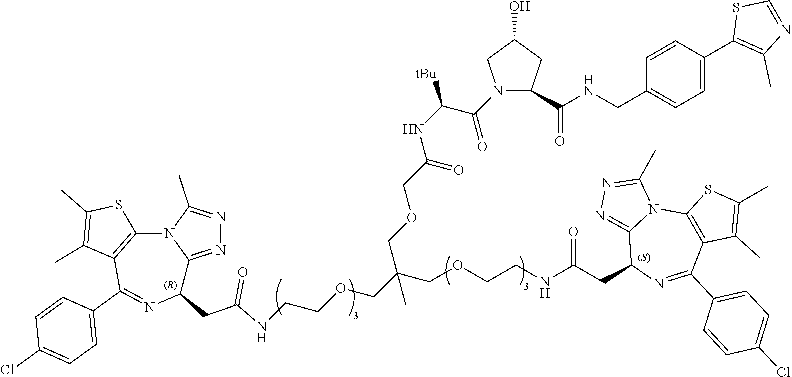

To a mixture of compound 32 (12 mg, 0.01 mmol) in MeOH (1 mL), were added 10% wt palladium on carbon (0.5 mg). The resulting reaction mixture was stirred under hydrogen atmosphere at r.t. overnight. The mixture was then filtered on a celite pad and evaporated. A pre stirred mixture of (−)-JQ1 carboxylic acid (4 mg, 0.01 mmol), HATU (4 mg, 0.01 mmol), N,N-diisopropylethylamine (5 μL, 0.03 mmol) in DMF (0.20 mL) was added to the concentrated crude. The mixture was stirred at r.t. for 1 h and purified by HPLC (5-95% CH₃CN in 0.1% aq. HCO₂H) to afford the title compound. Yield: 4.7 mg (31%).

¹H NMR (500 MHz, CDCl₃, mixture of diastereomers) δ (ppm)=8.70 (1H, s), 7.57-7.28 (14H, m), 7.20 (1H, m), 4.76 (1H, m), 4.72-4.60 (3H, m), 4.58-4.44 (2H, m), 4.43-4.33 (1H, m), 4.14-3.95 (3H, 3H), 3.69-3.25 (38H, m), 2.68-2.60 (6H, m), 2.50 (3H, s), 2.39 (6H, s), 2.36-2.29 (1H, m), 2.22-2.12 (1H, m), 1.68-1.61 (6H, m), 1.00-0.95 (9H, m), 0.92 (3H, s).

¹³C NMR (126 MHz, CDCl₃, mixture of diastereomers) δ (ppm)=171.46, 171.41, 170.89, 170.86, 170.7 (br), 170.6, 164.4 (br), 155.5, 150.4, 150.0, 148.1, 138.59, 138.55, 137.0 (br), 136.2 (br), 132.1 (br), 131.9, 131.2 (br), 131.11, 131.09, 130.47, 130.42, 130.1 (br), 129.33, 129.30, 128.7, 128.0, 73.8 (br), 70.92, 70.86, 70.61, 70.58, 70.4, 70.2 (br), 70.1, 69.9, 59.2, 59.1, 57.3, 56.8 (br), 53.9, 43.0, 41.0, 40.8, 39.3 (br), 38.0 (br), 36.7 (br), 35.59, 35.55, 29.7, 26.4, 17.75, 17.71, 15.95, 14.42, 14.39, 14.37, 13.1, 11.7

HRMS (ESI) for C₇₉H₉₉Cl₂N₁₄O₁₃S₃ [M+H⁺] calculated 1617.6050, obtained 1617.6025.

### Synthesis of Compound MN674

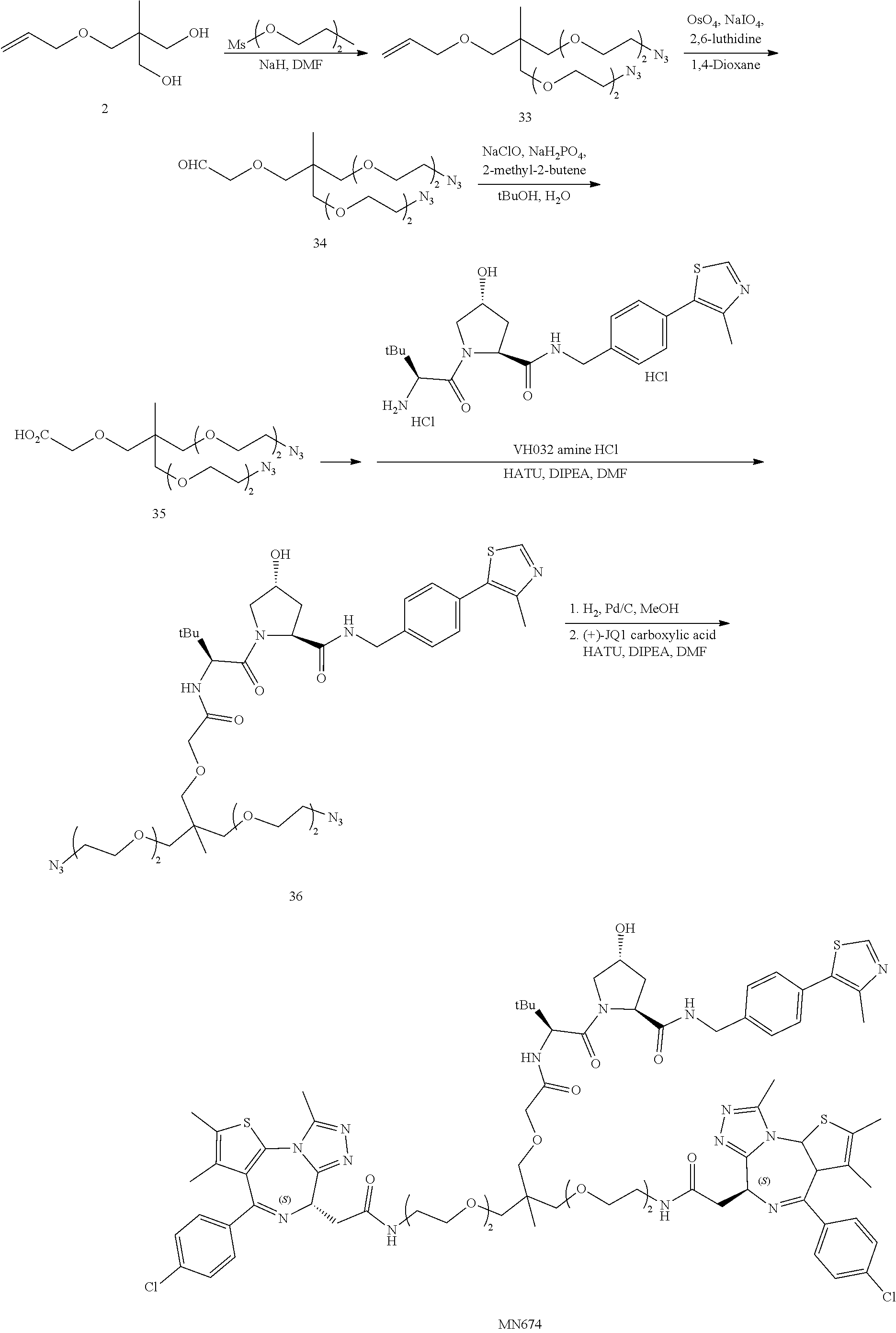

### 8-((allyloxy)methyl)-1,15-diazido-8-methyl-3,6,10,13-tetraoxapentadecane (33)

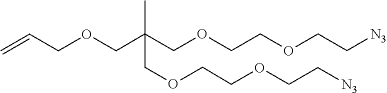 [SMILES: C=CCOCC(C)(COCCOCCN=[N+]=[N-])COCCOCCN=[N+]=[N-]]

2-((allyloxy)methyl)-2-methylpropane-1,3-diol 2 (350 mg, 2.19 mmol) was dissolved in DMF (5 mL) and cooled to 0° C. NaH (350 mg, 60% in oil, 8.75 mmol) was added and the reaction was stirred at 0° C. for 15 min. After that, 2-(2-azidoethoxy)ethyl methanesulfonate (1.4 g, 6.5 mmol) was added and the reaction was stirred at 60° C. overnight. The mixture was then filtered on a celite pad and concentrated. The crude material was purified by HPLC under acidic condition (5-95% CH₃CN in 0.1% aq. HCO₂H) to afford title compound. Yield: 507 mg (60%).

¹H NMR (500 MHz, CDCl₃) δ (ppm)=5.96-5.84 (1H, m), 5.29 (1H, m), 5.16 (1H, m), 4.00-3.95 (2H, m), 3.73-3.68 (4H, m), 3.68-3.63 (4H, m), 3.63-3.58 (4H, m), 3.42-3.36 (8H, m), 3.33 (2H, m), 0.98 (3H, s).

### 2-(3-(2-(2-azidoethoxy)ethoxy)-2-((2-(2-azidoethoxy)ethoxy)methyl)-2-methylpropoxy)acetaldehyde (34)

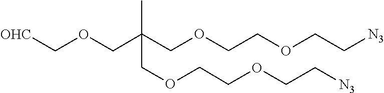 [SMILES: CC(COCC=O)(COCCOCCN=[N+]=[N-])COCCOCCN=[N+]=[N-]]

To a mixture of 8-((allyloxy)methyl)-1,15-diazido-8-methyl-3,6,10,13-tetraoxapentadecane 33 (507 mg, 1.31 mmol) in H₂O (4 mL) and 1,4-dioxane (14 mL), were added 2,6-luthidine (330 μL, 2.6 mmol), osmium tetroxide 4% in H₂O (180 μL, 0.01 mmol), sodium periodate (1.2 g, 0.21 mmol). The resulting reaction mixture was stirred at r.t. for 16 h. The reaction mixture was quenched with Na₂SO₃ (aq) and extracted with dichloromethane. The organic layer was concentrated and the remaining residue was purified by column chromatography to afford title compound. Yield: 425 mg (85%).

¹H NMR (500 MHz, CDCl₃) δ (ppm)=9.75 (1H, s), 4.02 (2H, s), 3.73-3.58 (14H, m), 3.46 (2H, m), 3.42-3.35 (10H, m), 1.01 (3H, s).

### 2-(3-(2-(2-azidoethoxy)ethoxy)-2-((2-(2-azidoethoxy)ethoxy)methyl)-2-methylpropoxy)acetic acid (35)

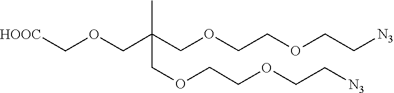 [SMILES: CC(COCCOCCN=[N+]=[N-])(COCCOCCN=[N+]=[N-])COCC(=O)O]

To a mixture of 2-(3-(2-(2-azidoethoxy)ethoxy)-2-((2-(2-azidoethoxy)ethoxy)methyl)-2-methylpropoxy)acetaldehyde 34 (425 mg, 1.1 mmol) in t-BuOH (15 mL) were added 2M 2-methyl-2-butene in THE (2.75 mL, 5.5 mmol), NaH₂PO₄ (132 mg, 1.1 mmol), sodium chlorite (500 mg, 4.4 mmol) in H₂O (3 mL). The resulting reaction mixture was stirred at r.t. for 16 h. The reaction mixture was concentrated and then diluted with NaOH (aq). The mixture was washed with MTBE and neutralized by 2M HCl. Extracted with dichloromethane, the organic layer was dried by Na₂SO₄ and concentrated. The remaining crude was used in next step without further purification. Yield: 422 mg (95%).

### (2S,4R)-1-((S)-15-azido-8-((2-(2-azidoethoxy)ethoxy)methyl)-2-(tert-butyl)-8-methyl-4-oxo-6,10,13-trioxa-3-azapentadecanoyl)-4-hydroxy-N-(4-(4-methylthiazol-5-yl)benzyl)pyrrolidine-2-carboxamide (36)

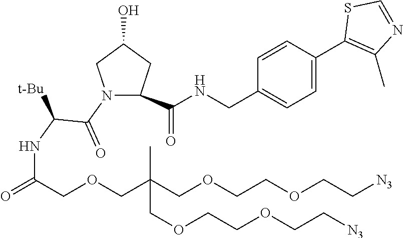 [SMILES: Cc1ncsc1-c1ccc(CNC(=O)[C@@H]2C[C@@H](O)CN2C(=O)[C@@H](NC(=O)COCC(C)(COCCOCCN=[N+]=[N-])COCCOCCN=[N+]=[N-])C(C)(C)C)cc1]

HATU (194 mg, 0.51 mmol) and N,N-diisopropylethylamine (270 μL) were added to 2-(3-(2-(2-azidoethoxy)ethoxy)-2-((2-(2-azidoethoxy)ethoxy)methyl)-2-methylpropoxy)acetic acid 35 (200 mg, 0.51 mmol) in DMF (2 mL). The resulting reaction mixture was stirred at r.t. for 2 min. VH032 amine hydrochloride (240 mg, 0.51 mmol) was added to the mixture. The mixture was stirred at r.t. for 1 h and purified by HPLC under acidic condition (5-95% CH₃CN in 0.1% aq. HCO₂H) to afford title compound. Yield: 312 mg (75%).

¹H NMR (500 MHz, CDCl₃) δ (ppm)=8.70 (1H, s), 7.43-7.33 (5H, m), 7.13 (1H, d, J=8.5 Hz), 4.77 (1H, t, J=7.8 Hz), 4.63-4.54 (2H, m), 4.47 (1H, d, J=8.7 Hz), 4.37 (1H, dd, J=5.5, 15.0 Hz), 4.19-4.13 (1H, m), 3.96 (2H, m), 3.71-3.53 (14H, m), 3.46-3.34 (10H, m), 2.63 (1H, m), 2.54 (3H, s), 2.14 (1H, m), 1.01 (3H, s), 0.97 (9H, s).

MS (ESI) for C₃₇H₅₇N₁₀O₉S [M+H⁺] calculated 817.4, obtained 817.9.

### N,N′-(8-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-8-methyl-3,6,10,13-tetraoxapentadecane-1,15-diyl)bis(2-((S)-4-(4-chlorophenyl)-2,3,9-trimethyl-6H-thieno[3,2-f][1,2,4]triazolo[4,3-a][1,4]diazepin-6-yl)acetamide) (MN674)

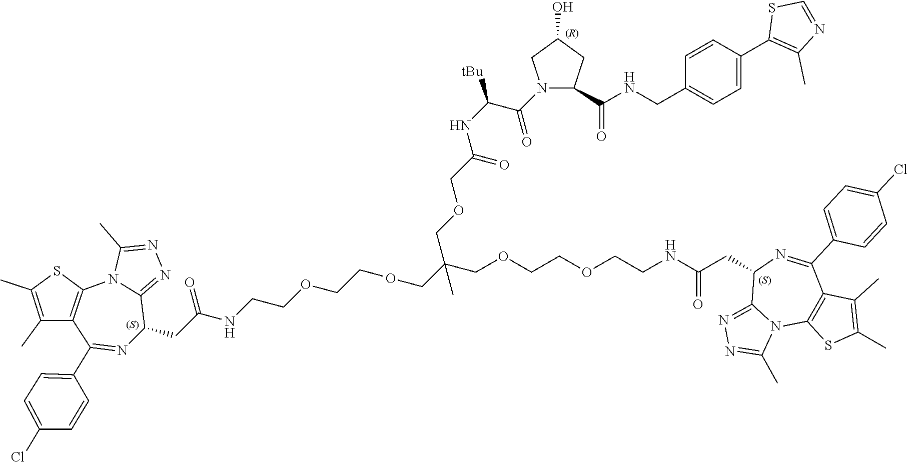

To a solution of (2S,4R)-1-((S)-15-azido-8-((2-(2-azidoethoxy)ethoxy)methyl)-2-(tert-butyl)-8-methyl-4-oxo-6,10,13-trioxa-3-azapentadecanoyl)-4-hydroxy-N-(4-(4-methylthiazol-5-yl)benzyl)pyrrolidine-2-carboxamide 36 (20 mg, 0.024 mmol) in MeOH (1 mL), were added 10% wt palladium on carbon (0.5 mg). The resulting reaction mixture was stirred under hydrogen atmosphere at r.t. overnight. The mixture was then filtered on a celite pad and evaporated. A pre stirred mixture of (+)-JQ1 carboxylic acid (20 mg, 0.05 mmol), HATU (19 mg, 0.05 mmol), N,N-diisopropylethylamine (20 μL) in DMF (0.5 mL) was added to the concentrated crude. The mixture was stirred at r.t. for 1 h and purified by HPLC (5-95% CH₃CN in 0.1% aq. HCO₂H) to afford the title compound. Yield: 13.5 mg (37%).

¹H NMR (500 MHz, CDCl₃) δ (ppm)=8.70 (1H, s), 7.67-7.61 (2H, m), 7.57 (1H, m), 7.44-7.30 (12H, m), 7.24 (1H, d, J=9.4 Hz), 4.89 (t, 1H, J=8.0 Hz), 4.75-4.63 (3H, m), 4.53 (1H, dd, J=6.4, 15.2 Hz), 4.46 (1H, m), 4.34 (1H, dd, J=5.5, 15.1 Hz), 4.28 (1H, d, J=15.7 Hz), 4.14 (1H, d, J=11.0 Hz), 4.07 (1H, d, J=15.7 Hz), 3.76-3.22 (31H, m), 2.64 (6H, s), 2.51 (3H, s), 2.40 (6H, s), 2.39 (1H, m), 2.18 (1H, m), 1.67 (6H, s), 1.02 (9H, s), 0.93 (3H, s).

¹³C NMR (126 MHz, CDCl₃) δ (ppm)=171.5, 171.2, 170.9, 170.7, 164.0, 163.9, 155.7, 150.3, 149.9, 148.3, 138.4, 136.8, 136.75, 136.5, 132.04, 132.02, 131.8, 131.04, 130.97, 129.99, 129.97, 129.4, 128.75, 128.73, 128.0, 73.8, 73.6, 73.4, 71.3, 70.9, 70.3, 70.2, 70.0, 58.9, 57.3, 56.5, 54.1, 53.5, 43.1, 41.1, 39.6, 38.3, 36.6, 35.4, 26.6, 26.4, 17.7, 16.0, 14.4, 13.4, 13.1, 11.8 HRMS (ESI) for C₇₅H₉₁C₁₂N₁₄O₁₁S₃ [M+H⁺] calculated 1529.5531, obtained 1529.5516.

### REFERENCES

1. Che, Y., Gilbert, A. M., Shanmugasundaram, V. & Noe, M. C. Inducing protein-protein interactions with molecular glues. Bioorg Med Chem Lett 28, 2585-2592 (2018).
2. Maniaci, C. & Ciulli, A. Bifunctional chemical probes inducing protein-protein interactions. Curr Opin Chem Biol 52, 145-156 (2019).
3. Burslem, G. M. & Crews, C. M. Proteolysis-Targeting Chimeras as Therapeutics and Tools for Biological Discovery. Cell 181, 102-114 (2020).
4. Petzold, G., Fischer, E. S. & Thoma, N. H. Structural basis of lenalidomide-induced CK1a degradation by the CRL4(CRBN) ubiquitin ligase. Nature 532, 127-130 (2016).
5. Matyskiela, M. E. et al. A novel cereblon modulator recruits GSPT1 to the CRL4(CRBN) ubiquitin ligase. Nature 535, 252-257 (2016).
6. Bussiere, D. E. et al. Structural basis of indisulam-mediated RBM39 recruitment to DCAF15 E3 ligase complex. Nat Chem Biol 16, 15-23 (2020).
7. Stabicki, M. et al. The CDK inhibitor CR8 acts as a molecular glue degrader that depletes cyclin K. Nature 359, eaao5902-5 (2020).
8. Mayor-Ruiz, C. et al. Rational discovery of molecular glue degraders via scalable chemical profiling. Nat Chem Biol 348, 1376 (2020).
9. Zengerle, M., Chan, K.-H. & Ciulli, A. Selective Small Molecule Induced Degradation of the BET Bromodomain Protein BRD4. ACS Chem Biol 10, 1770-1777 (2015).
10. Bondeson, D. P. et al. Catalytic in vivo protein knockdown by small-molecule PROTACs. Nat Chem Biol 11, 611-617 (2015).
11. Winter, G. E. et al. Phthalimide conjugation as a strategy for in vivo target protein degradation. Science 348, 1376-1381 (2015).
12. Salami, J. et al. Androgen receptor degradation by the proteolysis-targeting chimera ARCC-4 outperforms enzalutamide in cellular models of prostate cancer drug resistance. Commun Biol, 100 (2018).
13. Hu, J. et al. Discovery of ERD-308 as a Highly Potent Proteolysis Targeting Chimera (PROTAC) Degrader of Estrogen Receptor (ER). J. Med. Chem. 62, 1420-1442 (2019).
14. Qin, C. et al. Discovery of QCA570 as an Exceptionally Potent and Efficacious Proteolysis Targeting Chimera (PROTAC) Degrader of the Bromodomain and Extra-Terminal (BET) Proteins Capable of Inducing Complete and Durable Tumor Regression. J. Med. Chem. 61, 6685-6704 (2018).
15. Farnaby, W. et al. BAF complex vulnerabilities in cancer demonstrated via structure-based PROTAC design. Nat Chem Biol 15, 672-680 (2019).
16. Wang, S. et al. Uncoupling of PARP1 trapping and inhibition using selective PARP1 degradation. Nat Chem Biol 15, 1223-1231 (2019).
17. Zorba, A. et al. Delineating the role of cooperativity in the design of potent PROTACs for BTK. P Natl Acad Sci Usa 115, E7285-E7292 (2018).
18. Popow, J. et al. Highly Selective PTK2 Proteolysis Targeting Chimeras (PROTACs) to Probe Focal Adhesion Kinase Scaffolding Functions. J. Med. Chem. 62, acs.jmedchem.8b01826-2520 (2019).
19. Burslem, G. M. et al. The Advantages of Targeted Protein Degradation Over Inhibition: An RTK Case Study. Cell Chem Biol 25, 67-77.e3 (2018).
20. Bensimon, A. et al. Targeted Degradation of SLC Transporters Reveals Amenability of Multi-Pass Transmembrane Proteins to Ligand-Induced Proteolysis. Cell Chem Biol (2020). doi:10.1016/j.chembiol.2020.04.003
21. Gadd, M. S. et al. Structural basis of PROTAC cooperative recognition for selective protein degradation. Nat Chem Biol 13, 514-521 (2017).
22. Bondeson, D. P. et al. Lessons in PROTAC Design from Selective Degradation with a Promiscuous Warhead. Cell Chem Biol 25, 78-87.e5 (2018).
23. Olson, C. M. et al. Pharmacological perturbation of CDK9 using selective CDK9 inhibition or degradation. Nat Chem Biol 14, 163-170 (2018).
24. Tovell, H. et al. Design and Characterization of SGK3-PROTAC1, an Isoform Specific SGK3 Kinase PROTAC Degrader. ACS Chem Biol 14, 2024-2034 (2019).
25. Testa, A. et al. 3-Fluoro-4-hydroxyprolines: Synthesis, Conformational Analysis, and Stereoselective Recognition by the VHL E3 Ubiquitin Ligase for Targeted Protein Degradation. J Am Chem Soc 140, 9299-9313 (2018).
26. Han, X. et al. Discovery ofARD-69 as a Highly Potent Proteolysis Targeting Chimera (PROTAC) Degrader of Androgen Receptor (AR) for the Treatment of Prostate Cancer. J. Med. Chem. 62, 941-964 (2019).
27. Maniaci, C. et al. Homo-PROTACs: bivalent small-molecule dimerizers of the VHL E3 ubiquitin ligase to induce self-degradation. Nat Commun 8, 830 (2017).
28. Potjewyd, F. et al. Degradation of Polycomb Repressive Complex 2 with an EED-Targeted Bivalent Chemical Degrader. Cell Chem Biol 27, 47-56.e15 (2020).
29. Zoppi, V. et al. Iterative Design and Optimization of Initially Inactive Proteolysis Targeting Chimeras (PROTACs) Identify VZ185 as a Potent, Fast, and Selective von Hippel-Lindau (VHL) Based Dual Degrader Probe of BRD9 and BRD7. J. Med. Chem. 62, 699-726 (2019).
30. Crew, A. P. et al. Identification and Characterization of von Hippel-Lindau-Recruiting Proteolysis Targeting Chimeras (PROTACs) of TANK-Binding Kinase 1. J. Med. Chem. 61, 583-598 (2018).
31. Riching, K. M. et al. Quantitative Live-Cell Kinetic Degradation and Mechanistic Profiling of PROTAC Mode of Action. ACS Chem Biol 13, 2758-2770 (2018).
32. Roy, M. J. et al. SPR-Measured Dissociation Kinetics of PROTAC Ternary Complexes Influence Target Degradation Rate. ACS Chem Biol 14, 361-368 (2019).
33. Daniels, D. L., Riching, K. M. & Urh, M. Monitoring and deciphering protein degradation pathways inside cells. Drug Discovery Today: Technologies 31, 61-68 (2019).
34. Nowak, R. P. et al. Plasticity in binding confers selectivity in ligand-induced protein degradation article. Nat Chem Biol 14, 706-714 (2018).
35. Mammen, M., Choi, S.-K. & Whitesides, G. M. Polyvalent Interactions in Biological Systems: Implications for Design and Use of Multivalent Ligands and Inhibitors. Angew Chem Int Ed Engl 37, 2754-2794 (1998).
36. Fujisawa, T. & Filippakopoulos, P. Functions of bromodomain-containing proteins and their roles in homeostasis and cancer. Nat Rev Mol Cell Biol 8, 246-262 (2017).
37. Raina, K. et al. PROTAC-induced BET protein degradation as a therapy for castration-resistant prostate cancer. P Natl Acad Sci Usa 113, 7124-7129 (2016).
38. Winter, G. E. et al. BET Bromodomain Proteins Function as Master Transcription Elongation Factors Independent of CDK9 Recruitment. Mol Cell 67, 5-18.e19 (2017).
39. Frost, J. et al. Potent and selective chemical probe of hypoxic signalling downstream of HIF-α hydroxylation via VHL inhibition. Nat Commun 7, 13312 (2016).
40. Tanaka, M. et al. Design and characterization of bivalent BET inhibitors. Nat Chem Biol 12, 1089-1096 (2016).
41. Waring, M. J. et al. Potent and selective bivalent inhibitors of BET bromodomains. Nat Chem Biol 12, 1097-1104 (2016).
42. Zhu, X. F. et al. Knockdown of heme oxygenase-1 promotes apoptosis and autophagy and enhances the cytotoxicity of doxorubicin in breast cancer cells. Oncol Lett 10, 2974-2980 (2015).
43. Runcie, A. C. et al. Optimization of a ‘bump-and-hole’ approach to allele-selective BET bromodomain inhibition. Chem Sci 9, 2452-2468 (2018).
44. Smith, B. E. et al. Differential PROTAC substrate specificity dictated by orientation of recruited E3 ligase. Nat Commun 10, 131 (2019).
45. Hughes, S. J. & Ciulli, A. Molecular recognition of ternary complexes: a new dimension in the structure-guided design of chemical degraders. Essays Biochem 61, 505-516 (2017).
46. Fisher, S. L. & Phillips, A. J. Targeted protein degradation and the enzymology of degraders. Curr Opin Chem Biol 44, 47-55 (2018).
47. Lucas, X., Van Molle, I. & Ciulli, A. Surface Probing by Fragment-Based Screening and Computational Methods Identifies Ligandable Pockets on the von Hippel-Lindau (VHL) E3 Ubiquitin Ligase. J. Med. Chem. 61, 7387-7393 (2018).
48. Sun, Q. et al. Discovery of small molecules that bind to K-Ras and inhibit Sos-mediated activation. Angew Chem Int Ed Engl 51, 6140-6143 (2012).
49. Takahashi, D. et al. AUTACs: Cargo-Specific Degraders Using Selective Autophagy. Mol Cell 76, 797-810.e10 (2019).
50. Yamazoe, S. et al. Heterobifunctional Molecules Induce Dephosphorylation of Kinases-A Proof of Concept Study. J. Med. Chem. 63, 2807-2813 (2020).
51. Banik, S. M. et al. Lysosome-targeting chimaeras for degradation of extracellular proteins. Nature 584, 291-297 (2020).
52. Siriwardena, S. U. et al. Phosphorylation-Inducing Chimeric Small Molecules. J Am Chem Soc 142, 14052-14057 (2020).
53. Galdeano, C. et al. Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J. Med. Chem. 57, 8657-8663 (2014).
54. Girardini, M. et al. Cereblon versus VHL: Hijacking E3 Ligases against Each Other Using PROTACs. Bioorg. Med. Chem. 27, 2466-2479 (2019).

1. A compound of Formula I: 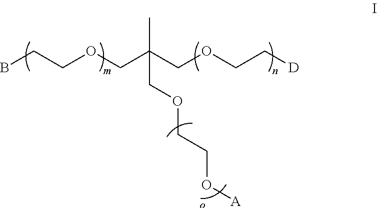
wherein B and D are each a ligand which binds to a target protein or polypeptide which is to be degraded by ubiquitin ligase;
wherein A is an E3 ubiquitin ligase protein binding ligand; and
wherein m, n and o are each independently selected from 0, 1, 2, 3, 4, 5 and 6;
or a pharmaceutically acceptable, salt, enantiomer, stereoisomer, hydrate, solvate, or polymorph thereof.

2. The compound according to claim 1, wherein m and n are independently selected from 2, 3, 4, 5 and 6.

3. The compound according to claim 1, wherein m and n are each independently selected from 2, 3 and 4.

4. The compound according to claim 1, wherein m and n are 3.

5. The compound according to claim 1, wherein o is selected from 0 and 1.

6. The compound according to claim 1, wherein at least one of:
B and D are each a chemical moiety which binds to a protein within the bromo- and Extra-terminal (BET) family of proteins;
B and D may each be a chemical moiety which induces degradation of the BRD2, BRD3, and/or BRD4 proteins within the bromo- and Extra-terminal (BET) family of proteins;
B and D are each independently selected from:

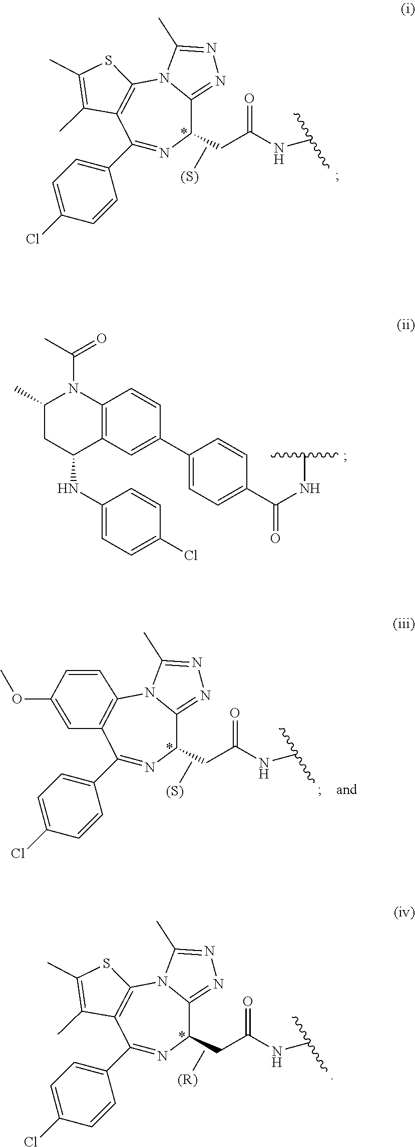

7. The compound according to claim 1, wherein at least one of B and D is

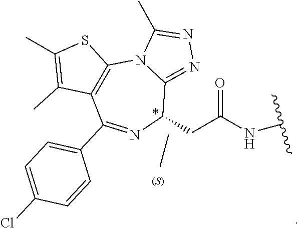

8. The compound according to claim 1, wherein both B and D are

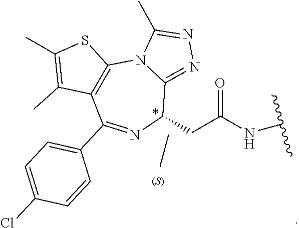

9. The compound according to claim 1, wherein A is selected from a von Hippel-Lindau (VHL)-E3 ubiquitin ligase binding ligand or a cereblon (CRBN)-E3 ubiquitin ligase ligand.

10. The compound according to claim 1, wherein A is selected from:

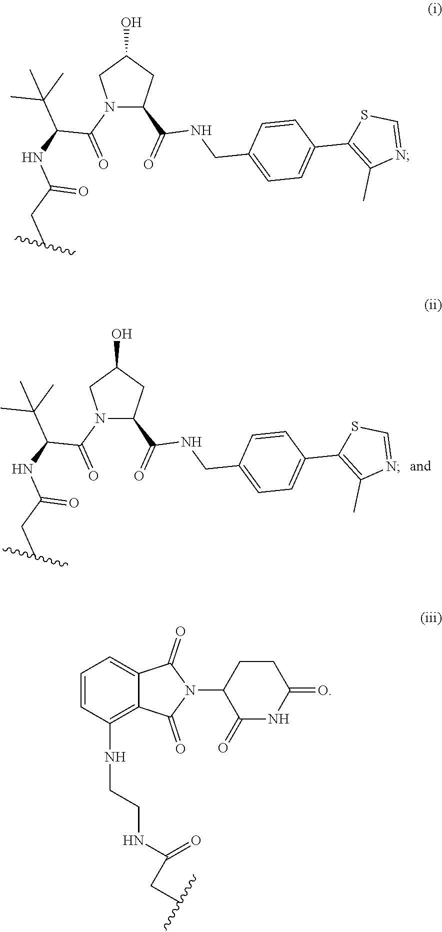 [SMILES: *CC(=O)N[C@H](C(=O)N1C[C@H](O)C[C@H]1C(=O)NCc1ccc(-c2scnc2C)cc1)C(C)(C)C ; *CC(=O)N[C@H](C(=O)N1C[C@@H](O)C[C@H]1C(=O)NCc1ccc(-c2scnc2C)cc1)C(C)(C)C ; *CC(=O)NCCNc1cccc2c1C(=O)N(C1CCC(=O)NC1=O)C2=O]

11. The compound according to claim 1, wherein A is selected from:

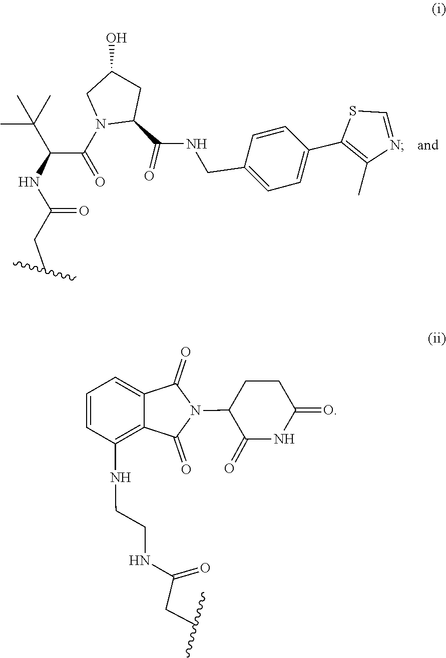 [SMILES: *CC(=O)N[C@H](C(=O)N1C[C@H](O)C[C@H]1C(=O)NCc1ccc(-c2scnc2C)cc1)C(C)(C)C ; "and" ; *CC(=O)NCCNc1cccc2c1C(=O)N(C1CCC(=O)NC1=O)C2=O]

12. The compound according to claim 1, wherein A is

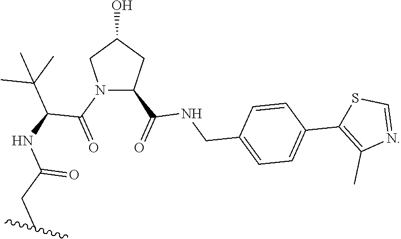 [SMILES: *CC(=O)N[C@H](C(=O)N1C[C@H](O)C[C@H]1C(=O)NCc1ccc(-c2scnc2C)cc1)C(C)(C)C]

13. The compound according to claim 1, wherein the compound of formula I has a formula IA: 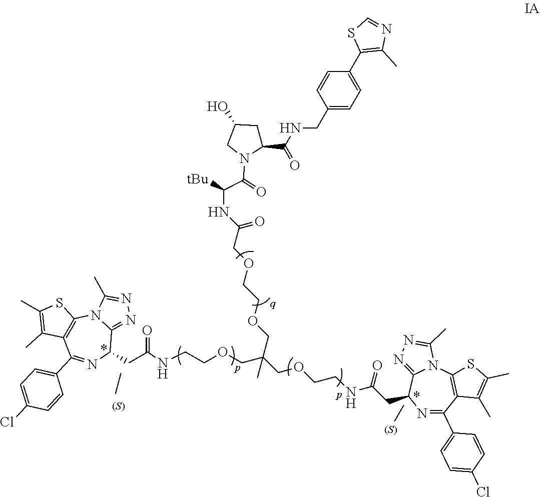
wherein p is selected from 2, 3, 4, 5 and 6; and
wherein q is selected from 0, 1, and 2;
or a pharmaceutically acceptable, salt, enantiomer, stereoisomer, hydrate, solvate, or polymorph thereof.

14. The compound according to claim 13, wherein p is selected from 2, 3 and 4.

15. The compound according to claim 13, wherein q is selected from 0 and 1.

16. The compound according to claim 1, wherein the compound of formula I has a formula IB: 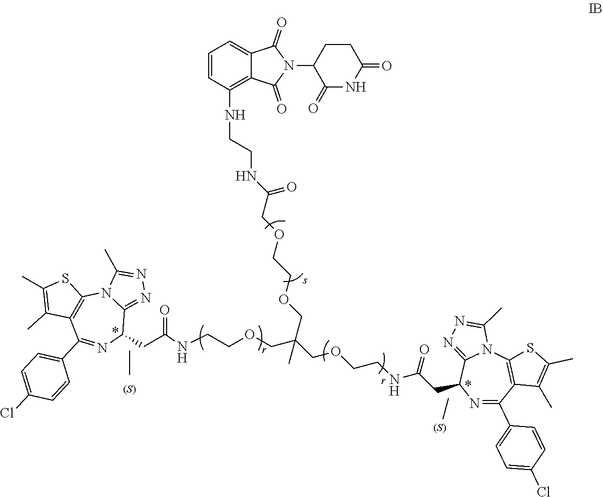
wherein r is selected from 2, 3, 4, 5 and 6; and
wherein s is selected from 0, 1, and 2;
or a pharmaceutically acceptable, salt, enantiomer, stereoisomer, hydrate, solvate, or polymorph thereof.

17. The compound according to claim 16, wherein at least one of:
r is selected from 2, 3 and 4;
s is selected from 0 and 1.

18. The compound according to claim 1, wherein the compound of formula I has a Formula IC: 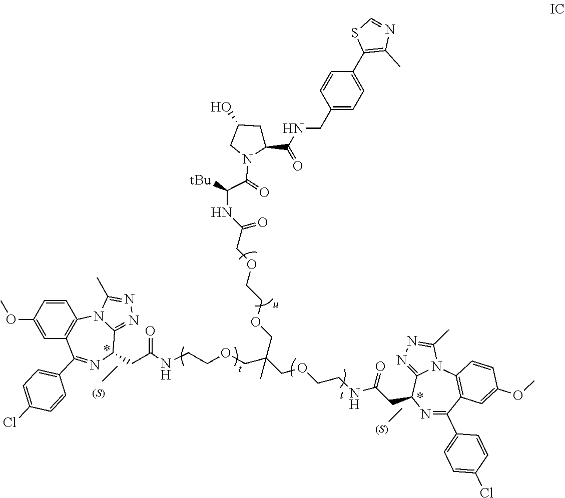
wherein t is selected from 2, 3, 4, 5 and 6; and
wherein u is selected from 0, 1, and 2;
or a pharmaceutically acceptable, salt, enantiomer, stereoisomer, hydrate, solvate, or polymorph thereof.

19. The compound according to claim 18, wherein at least one of:
t is selected from 2, 3 and 4;
u is selected from 0 and 1.

20. The compound according to claim 1, wherein the compound of formula I has formula ID: 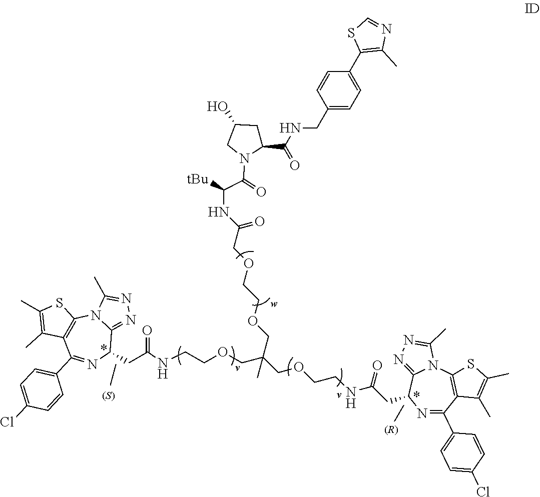
wherein v is selected from 2, 3, 4, 5 and 6; and
wherein w is selected from 0, 1, and 2;
or a pharmaceutically acceptable, salt, enantiomer, stereoisomer, hydrate, solvate, or polymorph thereof.

21. The compound according to claim 20, wherein at least one of:
v is selected from 2, 3 and 4;
w is selected from 0 and 1.

22. The compound according to claim 1, wherein the compound is selected from:
(i) N,N′-(11-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(ii) N,N′-(11-((2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(iii) N,N′-(14-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-14-methyl-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(iv) N,N′-(11-((2-((2-((2-(2,6-dioxopiperidin-3-yl)-1,3-dioxoisoindolin-4-yl)amino)ethyl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(v) N,N′-(11-((2-(2-((2-((2-(2,6-dioxopiperidin-3-yl)-1,3-dioxoisoindolin-4-yl)amino)ethyl)amino)-2-oxoethoxy)ethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(vi) N,N′-(14-((2-((2-((2-(2,6-dioxopiperidin-3-yl)-1,3-dioxoisoindolin-4-yl)amino)ethyl)amino)-2-oxoethoxy)methyl)-14-methyl-3,6,9,12,16,19,22,25-octaoxaheptacosane-1,27-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(vii) N,N′-(11-((2-(((S)-1-((2S,4S)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-11-methyl-3,6,9,13,16,19-hexaoxahenicosane-1,21-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
(viii) (2S,4R)-1-((20S)-20-(tert-butyl)-1-((R)-4-(4-chlorophenyl)-2,3,9-trimethyl-6H-thieno[3,2-f][1,2,4]triazolo[4,3-a][1,4]diazepin-6-yl)-14-(13-((S)-4-(4-chlorophenyl)-2,3,9-trimethyl-6H-thieno[3,2-f][1,2,4]triazolo[4,3-a][1,4]diazepin-6-yl)-12-oxo-2,5,8-trioxa-11-azatridecyl)-14-methyl-2,18-dioxo-6,9,12,16-tetraoxa-3,19-diazahenicosan-21-oyl)-4-hydroxy-N-(4-(4-methylthiazol-5-yl)benzyl)pyrrolidine-2-carboxamide; and
(ix) N,N′-(8-((2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)methyl)-8-methyl-3,6,10,13-tetraoxapentadecane-1,15-diyl)bis(2-((S)-4-(4-chlorophenyl)-2,3,9-trimethyl-6H-thieno[3,2-f][1,2,4]triazolo[4,3-a][1,4]diazepin-6-yl)acetamide);
or a pharmaceutically acceptable, salt, enantiomer, stereoisomer, hydrate, solvate, or polymorph thereof.

23. A pharmaceutical composition comprising the compound as defined in claim 1 and a pharmaceutically acceptable vehicle or diluent therefor.

24.-25. (canceled)

26. A method for the prophylaxis or treatment of a disease or condition in a subject, the method comprising administering to the subject the compound according to claim 1,
wherein the disease or condition is a cancer, a benign proliferative disorder, an infection, a non-infectious inflammatory event, an autoimmune disease, an inflammatory disease, a systemic inflammatory response syndrome, a viral infection, a viral disease, and/or an ophthalmological condition.

27. A method for the prophylaxis or treatment of a disease or condition in a subject, the method comprising administering to the subject the compound according to claim 1,
wherein the disease or condition is associated with deregulation of protein activity of one or more proteins within the Bromo- and Extra-terminal (BET) family of proteins BRD2, BRD3 and BRD4.
